(19) United States
(12) Patent Application Publication
(10) Pub. No.: US 2025/0034216 A1
(43) Pub. Date: Jan. 30, 2025

(54) STEREOSELECTIVE COVALENT LIGANDS FOR ONCOGENIC AND IMMUNOLOGICAL PROTEINS

(71) Applicant: The Scripps Research Institute, La Jolla, CA (US)

(72) Inventors: Benjamin CRAVATT, La Jolla, CA (US); Jarrett REMSBERG, San Diego, CA (US); Michael LAZEAR, San Diego, CA (US); Minoru YOKOYAMA, San Diego, CA (US); Michael SCHAFROTH, San Diego, CA (US); Daisuke OGASAWARA, San Diego, CA (US); Kristen DEMEESTER, San Diego, CA (US); Evert NJOMEN, Ypsilanti, MI (US); Bruno MELILLO, La Jolla, CA (US); Stuart SCHREIBER, Boston, MA (US)

(21) Appl. No.: 18/712,449

(22) PCT Filed: Nov. 21, 2022
(86) PCT No.: PCT/US2022/080272

§ 371 Date: May 22, 2024

Related U.S. Application Data
(60) Provisional application No. 63/282,128, filed on Nov. 22, 2021.

(51) Int. Cl. C07K 14/47

(52) U.S. Cl. CPC C07K 14/47

(57) ABSTRACT

Provided are in vivo engineered proteins. The engineered protein may be covalently bound to a ligand.

### CROSS REFERENCE

This application claims the benefit of U.S. Application No. 63/282,128, filed Nov. 22, 2021, which is hereby incorporated by reference in its entirety.

### STATEMENT AS TO FEDERALLY SPONSORED RESEARCH

This invention was made with the support of the United States government under Research Grant CA231991 awarded by the National Institutes of Health and under Research Grant PF-18-217-01-CDD awarded by the American Cancer Society. The government has certain rights in the invention.

### SUMMARY

Provided herein are in vivo engineered protein, comprising: a target protein comprising splicing factor 3B subunit 1 (SF3B1) or proteasome activator complex subunit 1 (PSME1), covalently bound to a small molecule ligand. In some embodiments, the ligand is covalently bound to a ligand binding site of the target protein. In some embodiments, the ligand is covalently bound to a cysteine residue of the ligand binding site. In some embodiments, the ligand comprises an exogenous Michael acceptor. In some embodiments, the exogenous Michael acceptor is an alkene or alkyne. In some embodiments, a sulfur atom at the cysteine residue undergoes the Michael reaction with a double bond of the exogenous Michael acceptor. In some embodiments, the ligand comprises an azetidine or tryptoline.

In some embodiment, the ligand comprises the structure of Formula (I), or a pharmaceutically acceptable salt or solvate thereof:

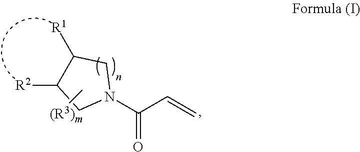

wherein,
R¹ is selected from the group consisting of H, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl;
R² is selected from the group consisting of substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl;
or R¹ and R² together with the atoms to which they are attached form a 5 to 10-membered heterocyclic ring A, optionally having one additional heteroatom moiety selected from NR⁴ or O, wherein A is optionally substituted; and
each R³ is independently H, —C(═O)OR⁵, —C(═O)N(R⁶)₂, —S(═O)₂R⁶, —S(═O)₂N(R⁶)₂, —N(R⁶)C(═O)R⁶, —N(R⁶)S(═O)₂R⁶, substituted or unsubstituted C₁-C₆ alkyl, substituted or unsubstituted aryl, or substituted or unsubstituted heteroaryl;
each R⁴ is independently H or C₁-C₆ alkyl;
each R⁵ is independently H, substituted or unsubstituted C₁-C₆alkyl, substituted or unsubstituted C₁-C₆haloalkyl, or substituted or unsubstituted C₁-C₁₀ heteroalkyl;
each R⁶ is independently H, substituted or unsubstituted C₁-C₆alkyl, substituted or unsubstituted C₁-C₆heteroalkyl, substituted or unsubstituted aryl, or substituted or unsubstituted heteroaryl;
or two R⁶ together with the atom to which they are attached form a 5 to 6-membered heterocyclic ring;
n is 0, 1, 2, or 3; and
m is 1, 2, or 3.

In some embodiments, the ligand has the structure of Formula (II), or a pharmaceutically acceptable salt or solvate thereof:

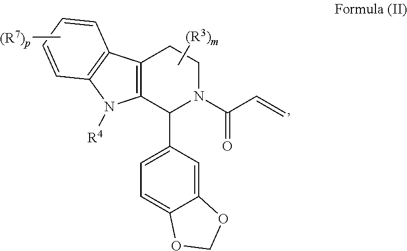

wherein,
each R⁷ is independently H, halogen, cyano, amino, hydroxyl, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, or substituted or unsubstituted C₁-C₆hydroxyalkyl; and
p is 1, 2, 3, or 4.

In some embodiments, the ligand has the structure of Formula (III), or a pharmaceutically acceptable salt or solvate thereof:

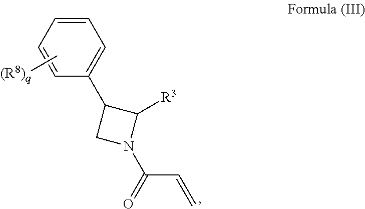

wherein,
each R⁷ is independently H, halogen, cyano, amino, hydroxyl, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, or substituted or unsubstituted C₁-C₆hydroxylkyl; and
q is 1, 2, 3, or 4.

In some embodiments, R¹ and R² together with the atoms to which they are attached form a 5 to 10-membered heterocyclic ring A, optionally having one additional heteroatom moiety selected from NR⁴ or O, wherein A is optionally substituted. In some embodiments, ring A is an 8 to 10-membered bicyclic heteroaryl optionally having one additional heteroatom selected from NR⁴. In some embodiments, R¹ is H and R² is selected from the group consisting of substituted or unsubstituted aryl or substituted or unsubstituted heteroaryl. In some embodiments, each R³ is independently —C(═O))R⁵ or —C(═O)N(R⁶)₂. In some embodiments, each R³ is independently substituted or unsubstituted aryl, or substituted or unsubstituted heteroaryl. In some embodiments, the ligand is selected from a compound described herein, or a pharmaceutically acceptable salt or solvate thereof.

In some embodiments, the ligand comprises an anti-cancer or immunomodulatory drug. In some embodiments, he target protein comprises SF3B1. In some embodiments, the target protein comprises an amino acid sequence having at least 90% sequence identity to SEQ ID NO: 1. In some embodiments, the ligand is covalently bound at amino acid position 1111 of the SF3B1. In some embodiments, the target protein comprises PSME1. In some embodiments, the target protein comprises an amino acid sequence having at least 90% sequence identity to SEQ ID NO: 2. In some embodiments, he ligand is covalently bound at or near a position on the PSME1 that interfaces with proteasome activator complex subunit 2 (PSME2). In some embodiments, the ligand is covalently bound at amino acid position 22 of the PSME1. In some embodiments, he ligand is covalently bound at amino acid position 106 of the PSME1. In some embodiments, the in vivo engineered protein comprising a neoantigen. In some embodiments, the in vivo engineered protein is formed in a cell.

### BRIEF DESCRIPTION OF THE DRAWINGS

FIG. 1A is a schematic for a SEC-MS proteomic platform. Cells are treated in situ with DMSO or electrophilic compounds, and lysates are fractionated by size exclusion chromatography into 5 fractions, each of which is digested and labelled with a tandem mass tag. Digests are then combined and analyzed by mass spectrometry. Elution profiles are deconvoluted for each protein detected.

FIG. 1B is a graphical comparison of the “mean elution time” from SEC-MS for 22Rv1 (x-axis) cells under control (DMSO) conditions vs. SEC data for U2OS cells (y-axis). Each dot represents a single protein detected in both experiments. A support vector regression line is displayed.

FIG. 1C includes chemical structures for elaborated stereoisomeric probe set 1. Dashed horizontal lines represent enantiomers, and dashed vertical or diagonal lines correspond to diastereomers.

FIG. 1D includes Chemical structures for elaborated stereoisomeric probe set 2. Dashed horizontal lines represent enantiomers, and dashed vertical or diagonal lines correspond to diastereomers.

FIG. 1E graphically represents size-exclusion shift scores (arbitrary units) plotted from profiling indicated probes at 20 μM in 22Rv1 cells for 3 h. The x-axis represents stereoselectivity between MY-1A and MY-1B (difference in SEC shifts from DMSO vs. MY-1A and DMSO vs. MY-1B). The y-axis represents stereoselectivity between MY-3A and MY-3B, n=2.

FIG. 1F graphically represents size-exclusion shift scores (a.u.) plotted from profiling indicated probes at 20 μM in 22Rv1 cells for 3 h. The x-axis represents stereoselectivity between MY-5A and MY-5B. The y-axis represents stereoselectivity between MY-7A and MY-7B, n=2.

FIG. 1G graphically represents SEC elution profiles for PSME1 and PSME2, displaying a strong stereoselective shift to lower molecular size fractions with MY-1B treatment. Data are shown as mean±SEM of the fractional distribution of PSME1 reporter ion intensities, n=2.

FIG. 1H graphically represents SEC elution profiles for DDX42, displaying a stereoselective shift to higher molecular size fractions after treatment with 20 μM MY-7A. Data are shown as mean SEM of the fractional distribution of reporter ion intensities, n=2.

FIG. 2A includes a heatmap showing stereoselectively engaged targets of stereoisomeric probe set 1. Cysteines quantified in at least two replicates with >66% engagement by one probe, and <25% by the remaining stereoisomers are shown. 22Rv1 cells were treated with 20 μM probe for 1 h, n=2-4 replicates.

FIG. 2B is a plot showing diastereomer probe profiling in 22Rv1 cells. MY-1A vs MY-1B shifts (x-axis) compared to MY-3A vs MY-3B shifts (y-axis). Proteins are colored based on whether they displayed stereoselective ligand engagement.

FIG. 2C is an image of a crystal structure of a PSME1/2 complex, highlighting the location of C22 (bottom inset), PSME1_C106 and PSME2_C91 (top inset) (PDB 7DRW).

FIG. 2D includes plots of ligandability data for cysteines on PSME1/2, highlighting that PSME1_C106 and C91 show an increase in IA-DTB reactivity after 1 h of 20 μM MY-1B treatment, while PSME1_C22 shows a reduction in IA-DTB labelling. Data are shown as mean±SD relative to DMSO, n=4.

FIG. 2E includes western blot images showing that PSME1_C22 is responsible for functional effects of MY-1B. The western blots are from 22Rv1 cells expressing recombinant FLAG-tagged PSME1 WT or C22A and treated with 10 μM probe for 3 h. C22A mutant rescues MY-1B induced a shift to lower molecular size fractions.

FIG. 2F includes plots of densitometry data from 22Rv1 cells expressing recombinant FLAG-PSME1 WT or C22A, treated with 10 μM MY-1A, MY-1B, or DMSO. Data are shown as mean±SD relative to DMSO, n=2.

FIG. 2G includes chemical structure of ‘click probes’, MY-11A (inactive) and MY-11B (active).

FIG. 2H includes western blot images showing MY-11B (active) alkyne probe labels recombinant PSME1 in a site- and stereo-specific manner. HEK293T lysates overexpressing WT or C22A mutant FLAG-tagged PSME1 were treated with 2.5 μM alkyne probe in vitro for 30 min followed by CuAAC reaction with rhodamine-azide for 1 h and analyzed by SDS-PAGE and western blot.

FIG. 3A includes chemical structures for butylamide analogues MY-45A (inactive), and MY-45B (active).

FIG. 3B includes an image of a representative gel-based ABPP experiment demonstrating that butynamide analogue MY-45B shows increased potency against recombinant FLAG-PSME1 in vitro. HEK293T lysates expressing recombinant FLAG-PSME1 WT or C22A were treated in vitro for 2 h with probe, followed by 30 min treatment with 2.5 μM MY-11B, CuAAC with rhodamine-azide for 1 h, and analyzed by SDS-PAGE.

FIG. 3C is a plot of a dose-response curve of MY-45B competition against MY-11B labeling, quantified by gel-based ABPP. Data are shown as mean SEM, n=6.

FIG. 3D graphically illustrates a SEC-MS elution profile for endogenous PSME1 in 22Rv1 cells after DMSO, MY-45A, or MY-45B treatment, 20 μM for 3 h. Data are shown as mean±SEM of the fractional distribution of reporter ion intensities, n=2.

FIG. 3E graphically illustrates functional effects of MY-45B treatment on antigen processing. Left: SIINFEKL MFI relative to DMSO at 1-, 2-, or 4-hours post-acid-wash. Right: MFI relative to DMSO 4 h after acid wash for both SIINFEKL and overall MHC-I for 5 μM MY-45A, 5 μM MY-45B, and 10 μM MG-132. Data are shown as mean±SD relative to DMSO, n=4. **, p<0.01, ***p<0.001 compared to MY-45A treatment.

FIG. 3F is a plot illustrating dose dependence of MY-45B impairment of antigen processing, 4 h post-acid-wash. Data are shown as mean±SD relative to DMSO, n=3. **, p<0.01 compared to MY-45A treatment.

FIG. 4A graphically depicts a molecular weight ladder fractionated by SEC-MS.

FIG. 4B shows mean pairwise Euclidean distances across constituent proteins for complexes annotated from the CORUM Core Complex dataset for both SEC-MS with 5 fractions (x-axis) and the data from Kirkwood et. al. with 40 fractions (y-axis). Each dot represents a single annotated protein complex—lower score represents tighter co-elution of complex members

FIG. 4C includes a comparison of the weighted average elution time for 22Rv1 (x-axis) for MCF7 (y-axis) cells under control (DMSO) conditions. Each dot represents a single protein, and proteins are colored based on their annotated molecular weight (UniProt). Large proteins elute only in higher-MW fractions; however, small proteins elute across all fractions, indicating the capture of native protein complexes.

FIG. 4D shows distribution of SEC shift scores (a.u.) for proteins detected in MY-1B or MY-7A treated samples.

FIG. 4E shows a PSME1 and PSME2 subunit arrangement in a heteroheptameric complex (PDB 7DRW).

FIG. 4F shows SEC elution profiles from 20s core proteasome subunits, demonstrating a minimal overlap with PSME1/2 elution profiles, and no change in 20s core proteasome elution times after treatment with compounds from diastereomer probe set 1. Data are shown as mean SEM of the fractional distribution of reporter ion intensities, n=2.

FIG. 5A is a plot indicating that MY-45B showed increased potency relative to MY-1B. IA-DTB cysteine engagement data for PSME1 Cys 22 from 22Rv1 cells treated with 5 μM probe in situ for 3 h. Data are shown as mean±SD relative to DMSO, n=8. ****, p<0.0001.

FIG. 5B is a heatmap of cysteines engaged (>66%) by either 5 μM MY-1B or MY-45B, demonstrating increased selectivity of MY-45B after 1 h treatment in 22Rv1 cells. Data are shown as mean, n=8.

FIG. 5C includes size-exclusion shift scores (arbitrary units) plotted from profiling MY-1A/B and MY-45A/B in 22Rv1 cells. The x-axis represents stereoselectivity between MY-1A and MY-1B. The y-axis represents stereoselectivity between MY-45A and MY-45B. Treatments were performed at 20 μM for 3 h. Data are shown as mean SEM of the fractional distribution of reporter ion intensities, n=2.

FIG. 5D includes images of immunoblots showing that MY-45B stereoselectively affects PSME1 elution profile; 5 μM treatment for 3 h in 22Rv1 cells overexpressing FLAG-PSME1 WT.

FIG. 5E is a schematic of an acid wash experiment where cells are treated in situ for 4 h, washed with citric acid or PBS for 2 min, then allowed to recover for up to 4 h prior to analysis by flow cytometry.

FIG. 5F includes plots demonstrating a flow cytometry gating strategy for SIINFEKL (APC) signal quantification.

FIG. 5G includes graphical cysteine engagement data for DDX42 after MY-7A treatment. 22Rv1 cells were treated with 20 μM probe for 3 h. Data are shown as mean±SD relative to DMSO, n=4.

FIG. 6A includes heatmap plots. The plot on the left is of protein abundance changes after treatment with diastereomer probe set 2 (20 μM for 8 h). The plot on the right shows MY-7A affected proteins, >33% decrease in expression, n=4-6.

FIG. 6B graphically indicates gene ontology enrichment for proteins stereoselectively degraded by MY-7A.

FIG. 6C is a line graph showing cell proliferation of 22Rv1 cells treated with varying concentrations of diastereomer probe set 2 for 72 h.

FIG. 6D includes chemical structures of MY-7A and MY-7B analogues WX-02-23 and WX-02-43 and click probes WX-01-10 and WX-01-12.

FIG. 6E is a line graph showing cell proliferation of 22Rv1 cells treated with varying concentrations of WX-02-23 or WX-02-43 for 72 h.

FIG. 6F is a plot where the x-axis includes log 2 enrichment scores for proteins competed by 5 μM WX-02-23 (2 h pretreatment) over 10 μM alkyne probe (1 h treatment), and the y-axis includes log₂ enrichment scores for proteins enriched with active over inactive probe (10 μM for 1 h).

FIG. 6G includes a graph of relative enrichment of SF3B1 by WX-01-10 or WX-01-12 after pre-treatment with DMSO, WX-02-23 or WX-02-43, from FIG. 6F. Relative enrichment represented the ratio between reporter ion intensities for each treatment group and the maximum reporter ion intensity across all channels.

FIG. 7A includes immunoblots showing confirmation of SF3B1 engagement in 22Rv1 cells. Top: Gel-based ABPP from cells treated with indicated compound for 24 h in situ (1 μM WX-02-23, WX-02-43, 10 nM Pladienolide B) followed by alkyne probe for 1 h (1 μM WX-01-10 or WX-01-12), lysis and CuAAC with rhodamine azide for 1 h. Bottom: anti-p27 western blot of the same samples.

FIG. 7B depicts aspects of a crystal structure of SF3B1-PHF5A with pladienolide B, highlighting the location of C1111 (PDB: 6EN4).

FIG. 7C depicts data from stereoselective blockade of IA-DTB labeling of SF3B1_C111 by WX-02-23.22Rv1 cells were treated with 20 μM WX-02-23 or WX-02-43 for 24 h and analyzed by targeted mass spectrometry. Data are shown as mean±SD relative to DMSO, n=3.

FIG. 7D depicts RNA-seq data from 5 μM WX-02-23 and 10 nM pladienolide B, after 8 hr treatment in 22Rv1 cells, data are shown as log 2 fold change relative to DMSO, n=4.

FIG. 7E depicts whole proteome data from 1 μM WX-02-23, 10 nM pladienolide B, and DMSO after 24 h treatment in 22Rv1 cells, data are shown as log 2 fold change relative to DMSO, n=4.

FIG. 7F is a heat map showing hierarchical clustering of RNA-seq genes that are regulated by pladienolide B treatment, n=4.

FIG. 7G shows examples of intron retention in Aurora Kinase B, and exon skipping in C2CD2L, induced by pladienolide B and WX-02-23.

FIG. 7H includes a summary of significant alternative splicing events induced by Pladienolide B, WX-02-43, and 1 μM or 5 μM WX-02-23 relative to DMSO as identified with rMATS by threshold of |PSI|>0.2 and FDR<0.05.

FIG. 7I depicts graphical data from co-immunoprecipitation of SF3B1 interacting proteins after 5 μM WX-02-23 or WX-02-43 treatment in situ for 3 h in HEK293T cells. Data presented as mean log 2 fold change±SD relative to DMSO showing differentially enriched proteins (>1.5 fold change increase/decrease), n=6-7.

FIG. 7J is an interactome map from STRING database filtered for proteins enriched as SF3B1 interactors by co-immunoprecipitation, as shown in FIG. 7I. Proteins with red boxes indicate interactions that are weakened by WX-02-23, and proteins in blue indicate significant increases in enrichment after WX-02-23 treatment.

FIG. 8A includes line graphs of stereoisomeric probe set 2 cell proliferation data for THP1 and Ramos cells.

FIG. 8B is a Venn diagram displaying proteins affected by MY-7A (>33% decrease in expression), or proteins annotated as “frequent responders.”

FIG. 8C includes chemical structures for non-covalent analogues of diastereomer probe set 2.

FIG. 8D is a plot of non-covalent analogues of diastereomer probe set 2 cell proliferation data for Ramos cells.

FIG. 8E is a plot of 22Rv1 cell proliferation data for alkyne probes WX-01-10 and WX-01-12.

FIG. 8F is a schematic for an alkyne-enrichment strategy leading to the identification of SF3B1.

FIG. 9A graphically depicts data from competition of WX-01-10 labelling by gel-based ABPP, as represented by FIG. 7A. Data are shown as mean±SD relative to DMSO, n=3. ****, p<0.0001, Significance was determined by one-way ANOVA with Dunnett's multiple comparisons test.

FIG. 9B shows a LC chromatogram of IA-DTB labeled SF3B1 peptide (aa 1110-1149) eluting at ˜38% acetonitrile.

FIG. 9C shows a representative chromatogram elution profile of targeted SF3B1 peptide fragment ions harboring C1111 (labeled in red) (dot product 0.86).

FIG. 9D includes graphical PRM data for additional cysteines present on SF3B1, none of which are engaged by WX-02-23.

FIG. 9E graphically depicts data from cysteine reactivity profiling following trypsin and GluC digestion confirming C1123 is not engaged by WX-02-23.

FIG. 9F includes cell proliferation data from Pladienolide B in 22Rv1, 72 h treatment.

FIG. 9G graphically depicts SE elution profiles of DDX42 and SF3B1 after WX-02-23 or WX-02-43 treatment; 20 μM probe for 3 h in situ in 22Rv1 cells, n=2.

FIG. 9H graphically depicts data from co-immunoprecipitation of SF3B1 interacting proteins after 5 μM WX-02-23 or 10 nM pladienolide B treatment in situ for 3 h in HEK293T cells. Data presented as mean log 2 fold change SD relative to DMSO showing differentially enriched proteins (>1.5 fold change increase/decrease), n=4-7.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

Despite the recent, human genetics-based discovery of many proteins that play fundamental roles in immunology, most proteins lack chemical probes and some may have even been considered undruggable. Disclosed herein are novel small-molecule ligands for target proteins, and drug-like covalent ligands for target proteins.

Describe herein are stereoselective covalent ligands that engage several oncogenic and immunological proteins of interest at good potency (low-μM) and selectivity in human cells. In some cases, these ligands are shown to affect the function of their target proteins. The ligands include: covalent inhibitors of splicing factor 3B subunit 1 (SF3B1) that alter the composition and function of the spliceosome or covalent inhibitors of proteasome activator complex subunit 1 (PSME1) that disrupt the structure and function of the PA28 regulatory complex of the proteasome.

The chemistry reported herein either represent the first small-molecule ligands for the protein targets of interest (e.g., PSME1) or the first drug-like covalent ligands for the protein targets of interest (e.g., SF3B1). The ligands may be useful as anti-cancer or immunomodulatory drugs for treating human disorders.

Also disclosed are target protein sites of ligand engagement. These sites may be useful as targets. For example, they may serve as drug targets for anti-cancer or immunomodulatory drugs.

Disclosed herein, in some embodiments, are in vivo engineered proteins, comprising: a target protein covalently bound to a small molecule ligand. Examples of target proteins include SF3B1 and PSME1. The covalent linkage may be through an amino acid residue such as a cysteine residue of the target protein.

### In Vivo Engineered Proteins

Disclosed herein, in some embodiments, are engineered proteins. Some embodiments include an in vivo engineered protein. Some embodiments include an in vivo modified protein. The in vivo engineered protein may include a modification. In some embodiments, the modification includes a ligand bound to the protein. The engineered protein may be non-naturally occurring.

In some embodiments, the in vivo engineered protein includes a target protein bound to a ligand. In some embodiments, the target protein is directly bound to the ligand. In some embodiments, the target protein is covalently bound to the ligand. In some embodiments, the target protein is non-covalently bound to the ligand. In some embodiments, the target protein is bound to the ligand in vivo.

Some embodiments relate to an in vivo engineered protein comprising a target protein covalently bound to a ligand. In some embodiments, the ligand is covalently bound to the target protein. In some embodiments, a cysteine of the target protein forms a covalent bond with an exogenous Michael acceptor of the ligand. In some embodiments, the ligand forma an adduct on the target protein. In some embodiments, the in vivo engineered protein is a ligand protein adduct.

### Target Proteins

Disclosed herein, in some embodiments, are target proteins. The target protein may be part of an in vivo engineered protein. The target protein may be bound to a ligand. For example, the target protein may be covalently bound to the ligand. Examples of target proteins include splicing factor 3B subunit 1 (SF3B1) or proteasome activator complex subunit 1 (PSME1). The target protein may include a protein described herein. For example, the target protein may include a protein in any of Tables 1-4.

In some embodiments, the target protein includes splicing factor 3B subunit 1 (SF3B1). The SF3B1 may be involved in splicing. For example, the SF3B1 may be a part of a splicing complex. The SF3B1 may include the amino acid sequence of SEQ ID NO: 1. In some embodiments, the SF3B1 includes an amino acid sequence at least 70% identical, at least 75% identical, at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical, to SEQ ID NO: 1. In some embodiments, the SF3B1 includes an amino acid sequence at least 99.1% identical, at least 99.2% identical, at least 99.3% identical, at least 99.4% identical, at least 99.5% identical, at least 99.6% identical, at least 99.7% identical, at least 99.8% identical, or at least 99.9% identical, to SEQ ID NO: 1. In some embodiments, the SF3B1 includes an amino acid sequence less than 70% identical, less than 75% identical, less than 80% identical, less than 85% identical, less than 90% identical, less than 91% identical, less than 92% identical, less than 93% identical, less than 94% identical, less than 95% identical, less than 96% identical, less than 97% identical, less than 98% identical, or less than 99% identical, to SEQ ID NO: 1. In some embodiments, the SF3B1 includes an amino acid sequence less than 99.1% identical, less than 99.2% identical, less than 99.3% identical, less than 99.4% identical, less than 99.5% identical, less than 99.6% identical, less than 99.7% identical, less than 99.8% identical, or less than 99.9% identical, to SEQ ID NO: 1. The SF3B1 may comprise a cysteine at amino acid position 1111. The cysteine may interact with or bind to the ligand. In some embodiments, the SF3B1 includes a full-length SF3B1 protein. For example, the SF3B1 may include 1304 amino acids. In some embodiments, the SF3B1 includes a fragment of SF3B1. In some embodiments, the SF3B1 fragment includes a functional fragment of SF3B1. The fragment of SF3B1 may include at least 100 amino acids, at least 200 amino acids, at least 300 amino acids, at least 400 amino acids, at least 500 amino acids, at least 600 amino acids, at least 700 amino acids, at least 800 amino acids, at least 900 amino acids, at least 1000 amino acids, at least 1100 amino acids, at least 1200 amino acids, or at least 1300 amino acids, of the full length SF3B1 protein. In some embodiments, the fragment of SF3B1 includes less than 100 amino acids, less than 200 amino acids, less than 300 amino acids, less than 400 amino acids, less than 500 amino acids, less than 600 amino acids, less than 700 amino acids, less than 800 amino acids, less than 900 amino acids, less than 1000 amino acids, less than 1100 amino acids, less than 1200 amino acids, or less than 1300 amino acids, of the full length SF3B1 protein.

In some embodiments, the target protein includes proteasome activator complex subunit 1 (PSME1). The PSME1 may be involved in protein degradation, for example, 26S proteasomal protein degradation. The PSME1 may include the amino acid sequence of SEQ ID NO: 2. In some embodiments, the PSME1 includes an amino acid sequence at least 70% identical, at least 75% identical, at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical, to SEQ ID NO: 2. In some embodiments, the PSME1 includes an amino acid sequence at least 99.5% identical to SEQ ID NO: 2. In some embodiments, the PSME1 includes an amino acid sequence less than 70% identical, less than 75% identical, less than 80% identical, less than 85% identical, less than 90% identical, less than 91% identical, less than 92% identical, less than 93% identical, less than 94% identical, less than 95% identical, less than 96% identical, less than 97% identical, less than 98% identical, or less than 99% identical, to SEQ ID NO: 2. In some embodiments, the PSME1 includes an amino acid sequence less than 99.5% identical to SEQ ID NO: 2. The PSME1 may comprise a cysteine at amino acid position 22 or position 106. The cysteine may interact with or bind to the ligand. In some embodiments, the PSME1 includes a full-length PSME1 protein. For example, the PSME1 may include 249 amino acids. In some embodiments, the PSME1 includes a fragment of PSME1. In some embodiments, the PSME1 fragment includes a functional fragment of PSME1. The fragment of PSME1 may include at least 50 amino acids, at least 100 amino acids, at least 150 amino acids, or at least 200 amino acids, of the full length PSME1 protein. In some embodiments, the fragment of PSME1 includes less than 50 amino acids, less than 100 amino acids, less than 150 amino acids, or less than 200 amino acids, of the full length PSME1 protein.

The target protein may include a ligand binding site. The ligand binding site may be part of a defined domain of the target protein. The ligand binding site may include an amino acid of the target protein. The ligand binding site may include multiple amino acids of the target protein. The ligand binding site may include a cysteine of the target protein. The ligand binding site may be at a surface of the target protein. The surface may be in contact with a solvent. For example, the surface may be in contact with the cytoplasm of a cell, or in contact with a biofluid inside a subject. The surface may be in contact with another protein.

The target protein may include a tag or label. For example, the target protein may include a FLAG epitope tag. In some embodiments, the target protein is radiolabeled.

### Ligands and Binding

Disclosed herein, in some embodiments, are ligands. The ligand may be part of an in vivo engineered protein. For example, the ligand may be bound to a target protein. The ligand may be bound (e.g. covalently bound) to a ligand binding site on the target protein.

In some embodiments, the ligand is exogenous. For example, the ligand may be exogenous to a cell. In some embodiments, the ligand is exogenous to a subject. In some embodiments, the ligand is administered or provided to the cell or to the subject, and then bind to the target protein in vivo.

In some embodiments, the ligand is a small molecule. The ligand may include a small molecule. An example of a small molecule is an organic compound having a molecular weight of less than 900 daltons. The ligand may have a molecular weight below 2500 daltons, below 2250 daltons, below 2000 daltons, below 1750 daltons, below 1500 daltons, or below 1250 daltons. The ligand may have a molecular weight below 1000 daltons, below 900 daltons, below 800 daltons, below 700 daltons, below 600 daltons, or below 500 daltons. The ligand may have a molecular weight greater than 2500 daltons, greater than 2250 daltons, greater than 2000 daltons, greater than 1750 daltons, greater than 1500 daltons, or greater than 1250 daltons. The ligand may have a molecular weight greater than 1000 daltons, greater than 900 daltons, greater than 800 daltons, greater than 700 daltons, greater than 600 daltons, or greater than 500 daltons.

In some embodiments, the ligand is a Michael receptor. In some embodiments, the ligand comprises an exogenous Michael acceptor. In some embodiments, the exogenous Michael acceptor comprises at least one double bond. In some embodiments, the exogenous Michael acceptor is an alkene or alkyne. In some embodiments, the exogenous Michael acceptor is an alkene. In some embodiments, the exogenous Michael acceptor is an alkyne. In some embodiments, the exogenous Michael acceptor comprise an acrylamide.

In some embodiments, the ligand comprises an azetidine or tryptoline core structure. In some embodiments, the ligand comprises an azetidine. In some embodiments, the ligand comprises a tryptoline.

In some embodiments, the ligand comprises the structure of Formula (I), or a pharmaceutically acceptable salt or solvate thereof:

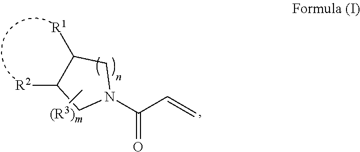

wherein,
R¹ is selected from the group consisting of H, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl;
R² is selected from the group consisting of substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl;
or R¹ and R² together with the atoms to which they are attached form a 5 to 10-membered heterocyclic ring A, optionally having one additional heteroatom moiety selected from NR⁴ or O, wherein A is optionally substituted; and
each R³ is independently H, —C(═O)OR⁵, —C(═O)N(R⁶)₂, —S(═O)₂R⁶, —S(═O)₂N(R⁶)₂, —N(R⁶)C(═O)R⁶, —N(R⁶)S(═O)₂R⁶, substituted or unsubstituted C₁-C₆ alkyl, substituted or unsubstituted aryl, or substituted or unsubstituted heteroaryl;
each R⁴ is independently H or C₁-C₆ alkyl;
each R⁵ is independently H, substituted or unsubstituted C₁-C₆alkyl, substituted or unsubstituted C₁-C₆haloalkyl, or substituted or unsubstituted C₁-C₁₀ heteroalkyl;
each R⁵ is independently H, substituted or unsubstituted C₁-C₆alkyl, substituted or unsubstituted C₁-C₆heteroalkyl, substituted or unsubstituted aryl, or substituted or unsubstituted heteroaryl;
or two R⁶ together with the atom to which they are attached form a 5 to 6-membered heterocyclic ring;
n is 0, 1, 2, or 3; and
m is 1, 2, or 3.

In some embodiments, R¹ is selected from the group consisting of substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl. In some embodiments, R¹ is selected from the group consisting of substituted or unsubstituted C₃-C₈cycloalkyl, substituted or unsubstituted C₂-C₇heterocycloalkyl, substituted or unsubstituted aryl, and substituted or unsubstituted heteroaryl. In some embodiments, R¹ is selected from the group consisting of substituted or unsubstituted aryl and substituted or unsubstituted heteroaryl. In some embodiments, R¹ is substituted or unsubstituted aryl. In some embodiments, R¹ is substituted or unsubstituted heteroaryl. In some embodiments, R¹ is H.

In some embodiments, R² is selected from the group consisting of substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl. In some embodiments, R² is selected from the group consisting of substituted or unsubstituted C₃-C₈cycloalkyl, substituted or unsubstituted C₂-C₇heterocycloalkyl, substituted or unsubstituted aryl, and substituted or unsubstituted heteroaryl. In some embodiments, R² is selected from the group consisting of substituted or unsubstituted aryl and substituted or unsubstituted heteroaryl. In some embodiments, R² is substituted or unsubstituted aryl. In some embodiments, R² is substituted or unsubstituted heteroaryl.

In some embodiments, R¹ and R² together with the atoms to which they are attached form a 5 to 10-membered heterocyclic ring A, optionally having one additional heteroatom moiety selected from NR⁴ or O, wherein A is optionally substituted. In some embodiments, R¹ and R² together with the atoms to which they are attached form a 5 to 10-membered monocyclic or bicyclic heteroaryl.

In some embodiments, ring A is a 8 to 10-membered bicyclic heteroaryl optionally having one additional heteroatom selected from NR⁴.

In some embodiments, n is 1. In some embodiments, n is 2. In some embodiments, n is 3. In some embodiments, n is 0.

In some embodiments, the ligand of Formula (I) has the structure of Formula (II), or a pharmaceutically acceptable salt or solvate thereof:

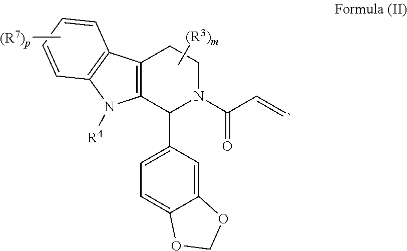

wherein,
each R⁷ is independently H, halogen, cyano, amino, hydroxyl, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, or substituted or unsubstituted C₁-C₆hydroxylkyl; and
p is 1, 2, 3, or 4.

In some embodiments, each R⁴ is independently H. In some embodiments, each R⁴ is independently C₁-C₆ alkyl. In some embodiments, each R⁴ is independently methyl, ethyl, or t-butyl.

In some embodiments, each R⁷ is independently halogen, cyano, amino, hydroxyl, substituted or unsubstituted C₁-C₆alkyl, substituted or unsubstituted C₁-C₆fluoroalkyl, substituted or unsubstituted C₁-C₆heteroalkyl, or substituted or unsubstituted C₁-C₆hydroxylkyl. In some embodiments, each R⁷ is independently substituted or unsubstituted C₁-C₆hydroxylkyl. In some embodiments, each R⁷ is independently substituted or unsubstituted C₂-C₆alkenyl or substituted or unsubstituted C₂-C₆alkynyl. In some embodiments, each R⁷ is independently substituted or unsubstituted C₂-C₆alkynyl. In some embodiments, each R⁷ is independently halogen, cyano, amino, hydroxyl, or C₁-C₆alkyl. In some embodiments, each R⁷ is independently halogen. In some embodiments, each R⁷ is independently chloro, bromo, or fluoro.

In some embodiments, each R⁷ is independently H.

In some embodiments, p is 1, 2, or 3. In some embodiments, p is 1 or 2. In some embodiments, p is 1. In some embodiments, p is 2. In some embodiments, p is 3. In some embodiments, p is 4.

In some embodiments, the ligand of Formula (I) has the structure of Formula (III), or a pharmaceutically acceptable salt or solvate thereof:

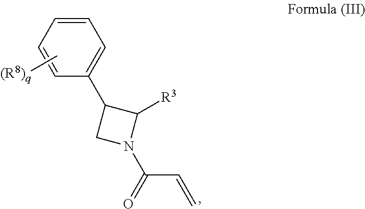

wherein,
each R⁸ is independently H, halogen, cyano, amino, hydroxyl, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, or substituted or unsubstituted C₁-C₆hydroxylkyl; and
q is 1, 2, 3, or 4.

In some embodiments, each R⁸ is independently halogen, cyano, amino, hydroxyl, substituted or unsubstituted C₁-C₆alkyl, substituted or unsubstituted C₁-C₆fluoroalkyl, substituted or unsubstituted C₁-C₆heteroalkyl, or substituted or unsubstituted C₁-C₆hydroxylkyl. In some embodiments, each R⁸ is independently substituted or unsubstituted C₂-C₆alkenyl or substituted or unsubstituted C₂-C₆alkynyl. In some embodiments, each R⁸ is independently substituted or unsubstituted C₂-C₆alkynyl. In some embodiments, each R⁸ is independently halogen, cyano, amino, hydroxyl, or C₁-C₆alkyl. In some embodiments, each R⁸ is independently halogen. In some embodiments, each R⁸ is independently chloro, bromo, or fluoro. In some embodiments, each R⁸ is independently chloro. In some embodiments, each R⁸ is independently bromo. In some embodiments, each R⁸ is independently fluoro.

In some embodiments, each R⁸ is independently H. In some embodiments, R⁸ is not H.

In some embodiments, q is 1, 2, or 3. In some embodiments, q is 1 or 2. In some embodiments, q is 1. In some embodiments, q is 2. In some embodiments, q is 3. In some embodiments, q is 4.

In some embodiments, each R³ is independently —C(═O)OR⁵, —C(═O)N(R⁶)₂, —S(═O)₂R⁶, —S(═O)₂N(R⁶)₂, —N(R⁶)C(═O)R⁶, —N(R⁶)S(═O)₂R⁶, substituted or unsubstituted C₁-C₆ alkyl, substituted or unsubstituted aryl, or substituted or unsubstituted heteroaryl.

In some embodiments, each R³ is independently —C(═O)OR⁵ or —C(═O)N(R⁶)₂. In some embodiments, each R³ is independently —C(═O)OR⁵, wherein R⁵ is H, substituted or unsubstituted C₁-C₆alkyl, substituted or unsubstituted C₁-C₆haloalkyl, or substituted or unsubstituted C₁-C₁₀ heteroalkyl. In some embodiments, R⁵ is H or substituted or unsubstituted C₁-C₆alkyl.

In some embodiments, each R³ is independently —C(═O)N(R⁶)₂, wherein each R⁶ is H, substituted or unsubstituted C₁-C₆alkyl, substituted or unsubstituted C₁-C₆heteroalkyl, or substituted or unsubstituted aryl. In some embodiments, each R⁶ is independently H or substituted or unsubstituted C₁-C₆alkyl. In some embodiments, each R⁶ is independently H, substituted or unsubstituted aryl or substituted or unsubstituted heteroaryl. In some embodiments, each R⁶ is independently H, substituted or unsubstituted aryl. In some embodiments, each R⁶ is independently H or substituted or unsubstituted heteroaryl. In some embodiments, two R⁶ together with the atom to which they are attached form a 5 to 6-membered heterocyclic ring. In some embodiments, two R⁶ together with the nitrogen atom to which they are attached form a morpholine ring.

In some embodiments, each R³ is independently substituted or unsubstituted aryl, or substituted or unsubstituted heteroaryl. In some embodiments, each R³ is independently substituted or unsubstituted aryl. In some embodiment, each R³ is independently substituted or unsubstituted phenyl. In some embodiments, each R³ is independently substituted or unsubstituted heteroaryl. In some embodiments, each R³ is independently substituted or unsubstituted heteroaryl optionally comprising 1-3 heteroatoms selected from N, O, or S. In some embodiments, each R³ is independently a 6 to 10 membered heteroaryl comprising 1-3 heteroatoms selected from O, N, or S. In some embodiments, each R³ is independently a 6 to 10 membered heteroaryl comprising 1-3 nitrogen atoms.

In some embodiments, m is 1 or 2. In some embodiments, m is 0. In some embodiments, m is 1. In some embodiments, m is 2. In some embodiments, m is 3.

In some embodiments, the ligand is

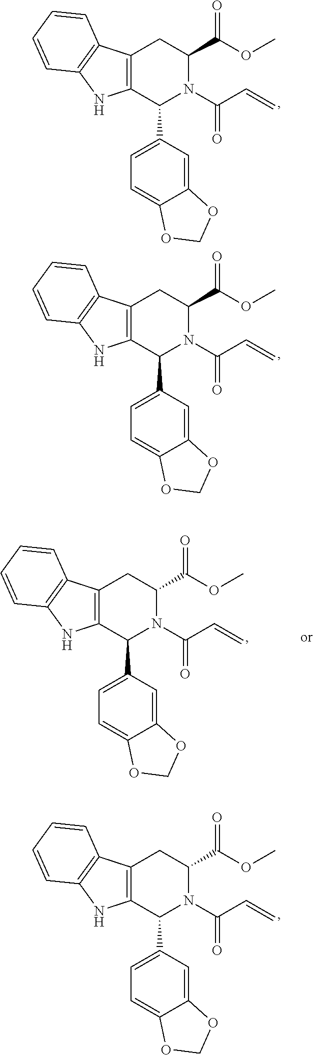

or a pharmaceutically acceptable salt or solvate thereof.

In some embodiments, the ligand is

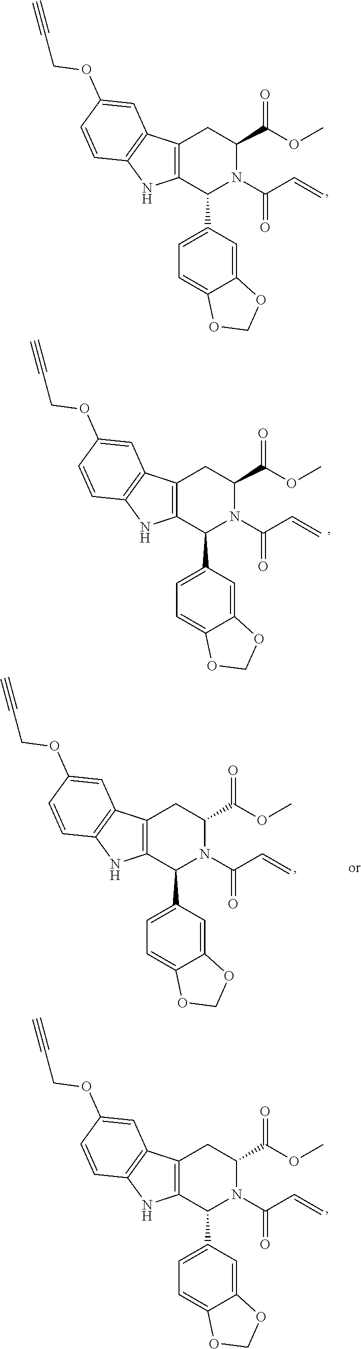

or a pharmaceutically acceptable salt or solvate thereof.

In some embodiments, the ligand is

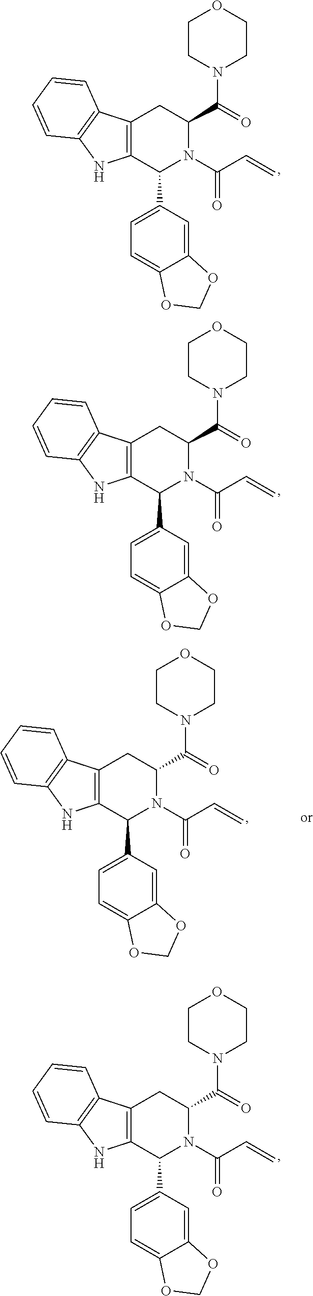

or a pharmaceutically acceptable salt or solvate thereof.

In some embodiments, the ligand is

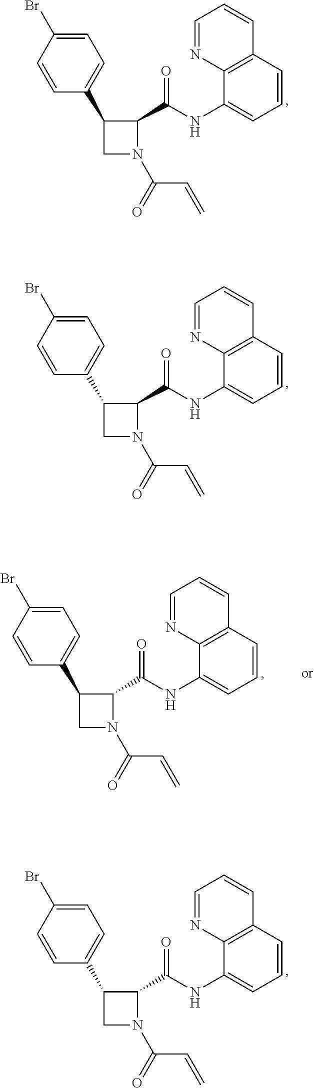 [SMILES: C=CC(=O)N1C[C@@H](c2ccc(Br)cc2)[C@H]1C(=O)Nc1cccc2cccnc12 ; C=CC(=O)N1C[C@H](c2ccc(Br)cc2)[C@H]1C(=O)Nc1cccc2cccnc12 ; C=CC(=O)N1C[C@@H](c2ccc(Br)cc2)[C@@H]1C(=O)Nc1cccc2cccnc12 ; C=CC(=O)N1C[C@H](c2ccc(Br)cc2)[C@@H]1C(=O)Nc1cccc2cccnc12]

or a pharmaceutically acceptable salt or solvate thereof.

In some embodiments, the ligand is

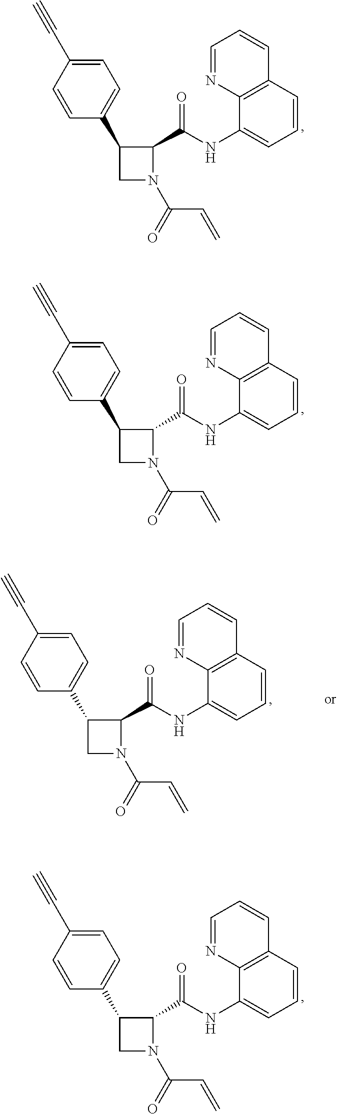 [SMILES: C#Cc1ccc([C@@H]2CN(C(=O)C=C)[C@@H]2C(=O)Nc2cccc3cccnc23)cc1 ; C#Cc1ccc([C@@H]2CN(C(=O)C=C)[C@H]2C(=O)Nc2cccc3cccnc23)cc1 ; C#Cc1ccc([C@H]2CN(C(=O)C=C)[C@@H]2C(=O)Nc2cccc3cccnc23)cc1 ; C#Cc1ccc([C@H]2CN(C(=O)C=C)[C@H]2C(=O)Nc2cccc3cccnc23)cc1]

or a pharmaceutically acceptable salt or solvate thereof.

In some embodiments, the ligand is

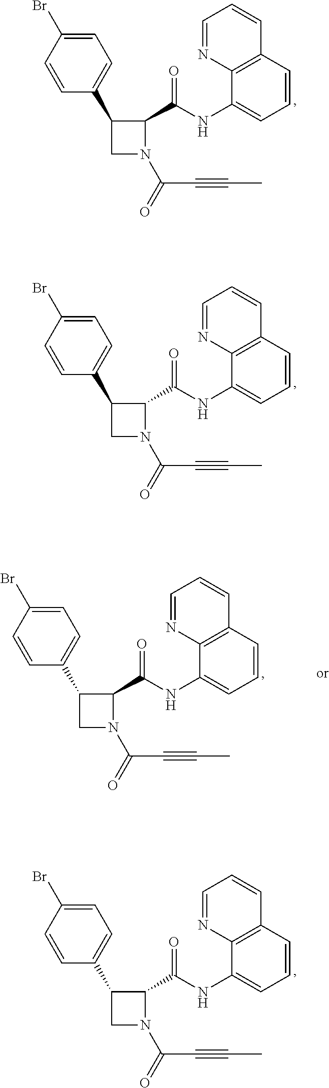 [SMILES: CC#CC(=O)N1C[C@@H](c2ccc(Br)cc2)[C@H]1C(=O)Nc1cccc2cccnc12 ; CC#CC(=O)N1C[C@@H](c2ccc(Br)cc2)[C@@H]1C(=O)Nc1cccc2cccnc12 ; CC#CC(=O)N1C[C@H](c2ccc(Br)cc2)[C@H]1C(=O)Nc1cccc2cccnc12 ; CC#CC(=O)N1C[C@H](c2ccc(Br)cc2)[C@@H]1C(=O)Nc1cccc2cccnc12]

or a pharmaceutically acceptable salt or solvate thereof.

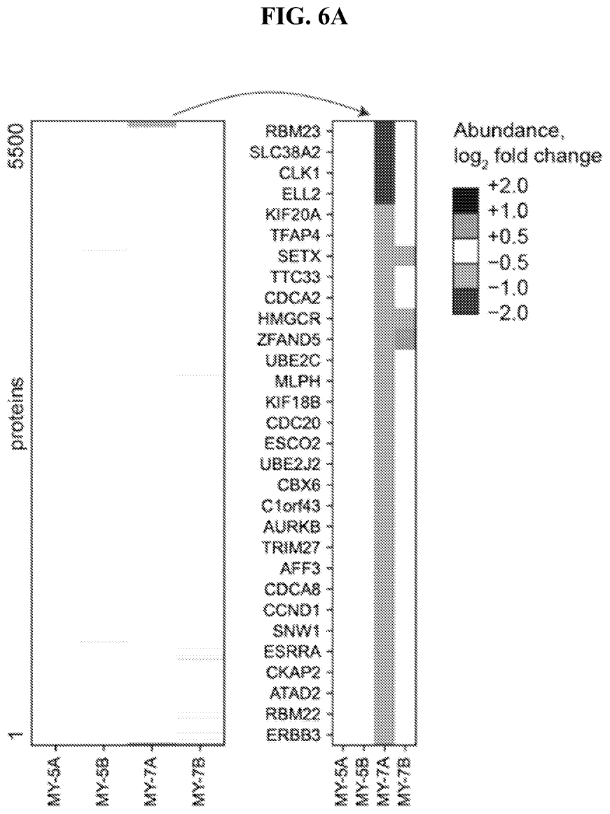

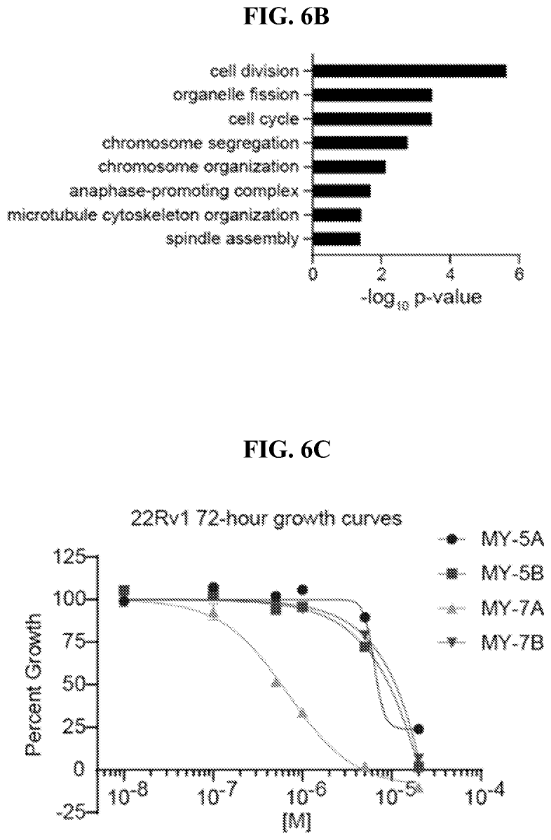

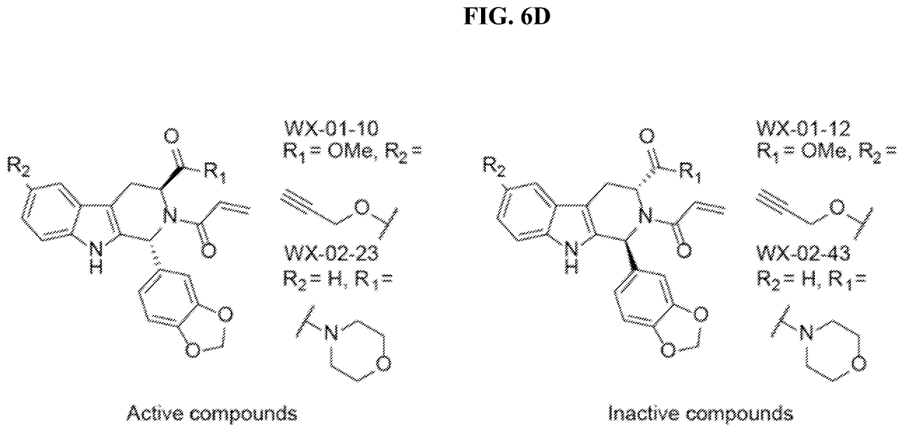

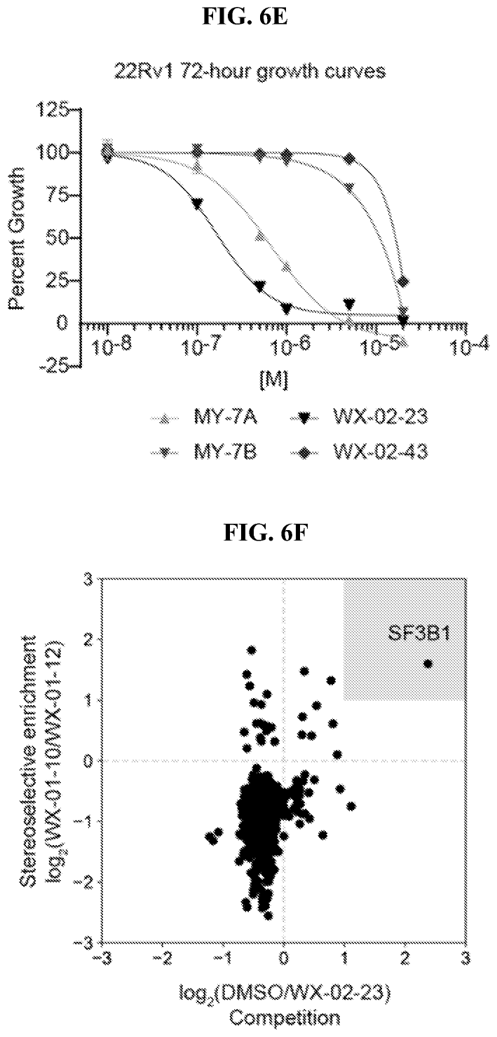

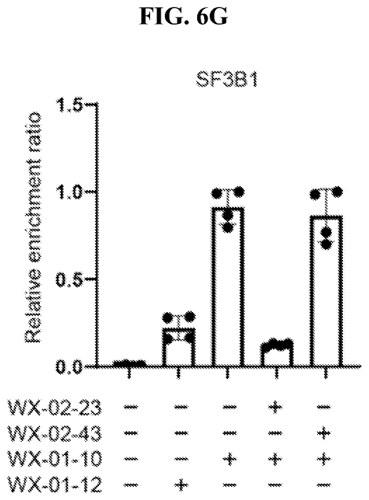

In some embodiments, the ligand is displayed in FIG. 6, or a pharmaceutically acceptable salt or solvate thereof.

The ligand may bind at a cysteine of the target protein. The ligand may bind at a cysteine position of a protein described herein. For example, the ligand binding site may include an amino acid or amino acid position in a protein included in any of Tables 1-4.

In some embodiments, a sulfur atom at a cysteine residue undergoes the Michael reaction with a double bond of the exogenous Michael acceptor, thereby forming a covalent bond.

In some embodiments, the protein is modified stereoselectively at a target amino acid residue (e.g. a cysteine). Without being bound by theory, in some instances, the protein is modified only at a specific amino acid residue. In some embodiments, the other amino acid residues are not modified.

In some embodiments, the exogenous Michael acceptor has a standard promiscuity of no greater than 60% at 500 μM. In some embodiments, the exogenous Michael acceptor has a standard promiscuity of no greater than 40% at 500 μM. In some embodiments, the exogenous Michael acceptor has a standard promiscuity that is smaller than 22% at 500 μM. In some embodiments, the exogenous Michael acceptor has a standard promiscuity of no greater than about 0.1%, 0.25%, 0.5%, 1%, 1.5%, 2%, 2.5%, 3%, 4%, 5%, 10%, 12.5%, 15%, 17.5%, 20%, 22.5%, 25%, 27.5%, 30%, 35%, 40%, 45%, 50%, 55%, 60%, 65%, 70%, 75%, 80%, 85%, 90%, or 95% at 500 μM. In some embodiments, the exogenous Michael acceptor has a standard promiscuity of in the range of about 0.1%-90%, about 0.5%-80%, about 1%-60%, or about 3%-50% at 500 μM.

In some embodiments, the target protein comprises SF3B1. In some embodiments, the ligand is covalently bound at amino acid position 1111 of the SF3B1 (e.g. at a cysteine at position 1111 of the SF3B1).

In some embodiments, the target protein comprises PSME1. In some embodiments, the ligand is covalently bound at or near a position on the PSME1 that interfaces with proteasome activator complex subunit 2 (PSME2). In some embodiments, the ligand is covalently bound at amino acid position 22 of the PSME1 (e.g. at a cysteine at position 22 of the PSME1). In some embodiment, the ligand is covalently bound at amino acid position 106 of the PSME1 (e.g. at a cysteine at position 106 of the PSME1).

### Ligand Synthesis

In some embodiments, the synthesis of compounds such as ligands described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in a formula or ligand as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure).

### Modification Methods

Some embodiments relate to method of modifying a target protein. For example, the method may include engineering a target protein. The modification or engineering may take place in vivo. The method may include contacting a target protein to a ligand receptor in vivo. In some embodiments, the method includes providing an effective amount of the ligand to allow the target protein to bind to the ligand. In some embodiments, the method includes providing an effective amount of the ligand to allow the target protein to undergo a Michael addition reaction.

### Cells, Analytical Techniques, and Instrumentation

In certain embodiments, also described herein are methods for profiling a target protein to determine a reactive or ligandable cysteine residue. In some instances, the methods comprising profiling a cell sample or a cell lysate sample comprising the target protein. In some embodiments, the cell sample or cell lysate sample comprising the target protein is obtained from cells of an animal. In some instances, the animal cell includes a cell from a marine invertebrate, fish, insects, amphibian, reptile, or mammal. In some instances, the mammalian cell is a primate, ape, equine, bovine, porcine, canine, feline, or rodent. In some instances, the mammal is a primate, ape, dog, cat, rabbit, ferret, or the like. In some cases, the rodent is a mouse, rat, hamster, gerbil, hamster, chinchilla, or guinea pig. In some embodiments, the bird cell is from a canary, parakeet or parrots. In some embodiments, the reptile cell is from a turtles, lizard or snake. In some cases, the fish cell is from a tropical fish. In some cases, the fish cell is from a zebrafish (e.g. Danino rerio). In some cases, the worm cell is from a nematode (e.g. C. elegans). In some cases, the amphibian cell is from a frog. In some embodiments, the arthropod cell is from a tarantula or hermit crab.

In some embodiments, the cell sample or cell lysate sample comprising the target protein is obtained from a mammalian cell. In some instances, the mammalian cell is an epithelial cell, connective tissue cell, hormone secreting cell, a nerve cell, a skeletal muscle cell, a blood cell, or an immune system cell.

Examples of mammalian cells include a 293A cell line, 293FT cell line, 293F cells, 293 H cells, HEK 293 cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, NC-HIMT cell line, or PC12 cell line.

In some instances, the cell sample or cell lysate sample comprising the target protein is obtained from cells of a tumor cell line. In some instances, the cell sample or cell lysate sample comprising the target protein is obtained from cells of a solid tumor cell line. In some instances, the solid tumor cell line is a sarcoma cell line. In some instances, the solid tumor cell line is a carcinoma cell line. In some embodiments, the sarcoma cell line is obtained from a cell line of alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, telangiectatic osteosarcoma.

In some embodiments, the carcinoma cell line is obtained from a cell line of adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, the cell sample or cell lysate sample comprising the target protein is obtained from cells of a hematologic malignant cell line. In some instances, the hematologic malignant cell line is a T-cell cell line. In some instances, B-cell cell line. In some instances, the hematologic malignant cell line is obtained from a T-cell cell line of: peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NKIT-cell lymphomas, or treatment-related T-cell lymphomas.

In some instances, the hematologic malignant cell line is obtained from a B-cell cell line of: acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the cell sample or cell lysate sample comprising the target protein is obtained from a tumor cell line. Exemplary tumor cell line includes, but is not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In some embodiments, the cell sample or cell lysate sample comprising the target protein is from any tissue or fluid from an individual. Samples include, but are not limited to, tissue (e.g. connective tissue, muscle tissue, nervous tissue, or epithelial tissue), whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In some embodiments, the cell sample or cell lysate sample is a tissue sample, such as a sample obtained from a biopsy or a tumor tissue sample. In some embodiments, the cell sample or cell lysate sample is a blood serum sample. In some embodiments, the cell sample or cell lysate sample is a blood cell sample containing one or more peripheral blood mononuclear cells (PBMCs). In some embodiments, the cell sample or cell lysate sample contains one or more circulating tumor cells (CTCs). In some embodiments, the cell sample or cell lysate sample contains one or more disseminated tumor cells (DTC, e.g., in a bone marrow aspirate sample).

In some embodiments, the cell sample or cell lysate sample comprising the target protein is obtained from the individual by any suitable means of obtaining the sample. For example, procedures for drawing and processing tissue sample such as from a needle aspiration biopsy may be employed to obtain a sample for use in the methods provided. For collection of such a tissue sample, a thin hollow needle may be inserted into a mass such as a tumor mass for sampling of cells.

### Sample Preparation and Analysis

In some embodiments, a target protein sample solution comprises a cell sample, a cell lysate sample, or a sample comprising a target protein. The sample solution may comprise isolated proteins that include the target protein. In some instances, the sample solution comprises a solution such as a buffer (e.g. phosphate buffered saline) or a media. In some embodiments, the media is an isotopically labeled media. In some instances, the sample solution is a cell solution.

In some embodiments, the target protein solution sample (e.g., cell sample, cell lysate sample, or comprising isolated proteins) is incubated with a ligand of the target protein for analysis of protein-probe interactions. In some instances, the solution sample (e.g., cell sample, cell lysate sample, or comprising isolated proteins) is further incubated in the presence of an additional compound probe prior to addition of the ligand. In other instances, the solution sample (e.g., cell sample, cell lysate sample, or comprising isolated proteins) is further incubated with a ligand, in which the ligand does not contain a photoreactive moiety and/or an alkyne group. In such instances, the solution sample is incubated with a probe and a ligand for competitive protein profiling analysis.

In some cases, the cell sample or the cell lysate sample comprising the target protein is compared with a control. In some cases, a difference is observed between a set of probe protein interactions between the sample and the control. In some instances, the difference correlates to the interaction between the small molecule fragment and the proteins.

In some embodiments, one or more methods are utilized for labeling a target protein solution sample (e.g. cell sample, cell lysate sample, or comprising isolated proteins) for analysis of probe protein interactions. In some instances, a method comprises labeling the sample (e.g. cell sample, cell lysate sample, or comprising isolated proteins) with an enriched media. In some cases, the sample (e.g. cell sample, cell lysate sample, or comprising isolated proteins) is labeled with isotope-labeled amino acids, such as ³C or ¹⁵N-labeled amino acids. In some cases, the labeled sample is further compared with a non-labeled sample to detect differences in probe protein interactions between the two samples. In some instances, this difference is a difference of a target protein and its interaction with a small molecule ligand in the labeled sample versus the non-labeled sample. In some instances, the difference is an increase, decrease or a lack of protein-probe interaction in the two samples. In some instances, the isotope-labeled method is termed SILAC, stable isotope labeling using amino acids in cell culture.

In some embodiments, a method comprises incubating a solution sample (e.g. cell sample, cell lysate sample, or comprising isolated proteins) with a labeling group (e.g., an isotopically labeled labeling group) to tag one or more proteins of interest for further analysis. In such cases, the labeling group comprises a biotin, a streptavidin, bead, resin, a solid support, or a combination thereof, and further comprises a linker that is optionally isotopically labeled. As described above, the linker can be about 5, 6, 7, 8, 9, 10, 11, 12, 13, 14, 15 or more residues in length and might further comprise a cleavage site, such as a protease cleavage site (e.g., TEV cleavage site). In some cases, the labeling group is a biotin-linker moiety, which is optionally isotopically labeled with ¹³C and ¹⁵N atoms at one or more amino acid residue positions within the linker. In some cases, the biotin-linker moiety is a isotopically-labeled TEV-tag.

In some embodiments, an isotopic reductive dimethylation (ReDi) method is utilized for processing a sample. In some cases, the ReDi labeling method involves reacting peptides with formaldehyde to form a Schiff base, which is then reduced by cyanoborohydride. This reaction dimethylates free amino groups on N-termini and lysine side chains and monomethylates N-terminal prolines. In some cases, the ReDi labeling method comprises methylating peptides from a first processed sample with a “light” label using reagents with hydrogen atoms in their natural isotopic distribution and peptides from a second processed sample with a “heavy” label using deuterated formaldehyde and cyanoborohydride. Subsequent proteomic analysis (e.g., mass spectrometry analysis) based on a relative peptide abundance between the heavy and light peptide version might be used for analysis of probe-protein interactions.

In some embodiments, isobaric tags for relative and absolute quantitation (iTRAQ) method is utilized for processing a sample. In some cases, the iTRAQ method is based on the covalent labeling of the N-terminus and side chain amines of peptides from a processed sample. In some cases, reagent such as 4-plex or 8-plex is used for labeling the peptides.

In some embodiments, the probe-protein complex is further conjugated to a chromophore, such as a fluorophore. In some instances, the probe-protein complex is separated and visualized utilizing an electrophoresis system, such as through a gel electrophoresis, or a capillary electrophoresis. Exemplary gel electrophoresis includes agarose based gels, polyacrylamide based gels, or starch based gels. In some instances, the probe-protein is subjected to a native electrophoresis condition. In some instances, the probe-protein is subjected to a denaturing electrophoresis condition.

In some instances, the probe-protein after harvesting is further fragmentized to generate protein fragments. In some instances, fragmentation is generated through mechanical stress, pressure, or chemical means. In some instances, the protein from the probe-protein complexes is fragmented by a chemical means. In some embodiments, the chemical means is a protease. Exemplary proteases include, but are not limited to, serine proteases such as chymotrypsin A, penicillin G acylase precursor, dipeptidase E, DmpA aminopeptidase, subtilisin, prolyl oligopeptidase, D-Ala-D-Ala peptidase C, signal peptidase I, cytomegalovirus assemblin, Lon-A peptidase, peptidase Clp, Escherichia coli phage KiF endosialidase CIMCD self-cleaving protein, nucleoporin 145, lactoferrin, murein tetrapeptidase LD-carboxypeptidase, or rhomboid-1; threonine proteases such as ornithine acetyltransferase; cysteine proteases such as TEV protease, amidophosphoribosyltransferase precursor, gamma-glutamyl hydrolase (Rattus norvegicus), hedgehog protein, DmpA aminopeptidase, papain, bromelain, cathepsin K, calpain, caspase-1, separase, adenain, pyroglutamyl-peptidase I, sortase A, hepatitis C virus peptidase 2, sindbis virus-type nsP2 peptidase, dipeptidyl-peptidase VI, or DeSI-1 peptidase; aspartate proteases such as beta-secretase 1 (BACE1), beta-secretase 2 (BACE2), cathepsin D, cathepsin E, chymosin, napsin-A, nepenthesin, pepsin, plasmepsin, presenilin, or renin; glutamic acid proteases such as AfuGprA; and metalloproteases such as peptidase_M48.

In some instances, the fragmentation is a random fragmentation. In some instances, the fragmentation generates specific lengths of protein fragments, or the shearing occurs at particular sequence of amino acid regions.

In some instances, the protein fragments are further analyzed by a proteomic method such as by liquid chromatography (LC) (e.g. high performance liquid chromatography), liquid chromatography-mass spectrometry (LC-MS), matrix-assisted laser desorption/ionization (MALDI-TOF), gas chromatography-mass spectrometry (GC-MS), capillary electrophoresis-mass spectrometry (CE-MS), or nuclear magnetic resonance imaging (NMR).

In some embodiments, the LC method is any suitable LC methods well known in the art, for separation of a sample into its individual parts. This separation occurs based on the interaction of the sample with the mobile and stationary phases. Since there are many stationary/mobile phase combinations that are employed when separating a mixture, there are several different types of chromatography that are classified based on the physical states of those phases. In some embodiments, the LC is further classified as normal-phase chromatography, reverse-phase chromatography, size-exclusion chromatography, ion-exchange chromatography, affinity chromatography, displacement chromatography, partition chromatography, flash chromatography, chiral chromatography, and aqueous normal-phase chromatography.

In some embodiments, the LC method is a high performance liquid chromatography (HPLC) method. In some embodiments, the HPLC method is further categorized as normal-phase chromatography, reverse-phase chromatography, size-exclusion chromatography, ion-exchange chromatography, affinity chromatography, displacement chromatography, partition chromatography, chiral chromatography, and aqueous normal-phase chromatography.

In some embodiments, the HPLC method of the present disclosure is performed by any standard techniques well known in the art. Exemplary HPLC methods include hydrophilic interaction liquid chromatography (HILIC), electrostatic repulsion-hydrophilic interaction liquid chromatography (ERLIC) and reverse phase liquid chromatography (RPLC).

In some embodiments, the LC is coupled to a mass spectroscopy as a LC-MS method. In some embodiments, the LC-MS method includes ultra-performance liquid chromatography-electrospray ionization quadrupole time-of-flight mass spectrometry (UPLC-ESI-QTOF-MS), ultra-performance liquid chromatography-electrospray ionization tandem mass spectrometry (UPLC-ESI-MS/MS), reverse phase liquid chromatography-mass spectrometry (RPLC-MS), hydrophilic interaction liquid chromatography-mass spectrometry (HILIC-MS), hydrophilic interaction liquid chromatography-triple quadrupole tandem mass spectrometry (HILIC-QQQ), electrostatic repulsion-hydrophilic interaction liquid chromatography-mass spectrometry (ERLIC-MS), liquid chromatography time-of-flight mass spectrometry (LC-QTOF-MS), liquid chromatography-tandem mass spectrometry (LC-MS/MS), multidimensional liquid chromatography coupled with tandem mass spectrometry (LC/LC-MS/MS). In some instances, the LC-MS method is LC/LC-MS/MS. In some embodiments, the LC-MS methods of the present disclosure are performed by standard techniques well known in the art.

In some embodiments, the GC is coupled to a mass spectroscopy as a GC-MS method. In some embodiments, the GC-MS method includes two-dimensional gas chromatography time-of-flight mass spectrometry (GC*GC-TOFMS), gas chromatography time-of-flight mass spectrometry (GC-QTOF-MS) and gas chromatography-tandem mass spectrometry (GC-MS/MS).

In some embodiments, CE is coupled to a mass spectroscopy as a CE-MS method. In some embodiments, the CE-MS method includes capillary electrophoresis-negative electrospray ionization-mass spectrometry (CE-ESI-MS), capillary electrophoresis-negative electrospray ionization-quadrupole time of flight-mass spectrometry (CE-ESI-QTOF-MS) and capillary electrophoresis-quadrupole time of flight-mass spectrometry (CE-QTOF-MS).

In some embodiments, the nuclear magnetic resonance (NMR) method is any suitable method well known in the art for the detection of one or more cysteine binding proteins or protein fragments disclosed herein. In some embodiments, the NMR method includes one dimensional (1D) NMR methods, two dimensional (2D) NMR methods, solid state NMR methods and NMR chromatography. Exemplary 1D NMR methods include ¹Hydrogen, ¹³Carbon, ¹⁵Nitrogen, ¹⁷Oxygen, ¹⁹Fluorine, ³¹Phosphorus, ³⁹Potassium, ²³Sodium, ³³Sulfur, ⁸⁷Strontium, ²⁷Aluminium, ⁴³Calcium, ³⁵Chlorine, ³⁷Chlorine, ⁶³Copper, ⁶⁵Copper, ⁵⁷Iron, ²⁵Magnesium, ¹⁹⁹Mercury or ⁶⁷Zinc NMR method, distortionless enhancement by polarization transfer (DEPT) method, attached proton test (APT) method and 1D-incredible natural abundance double quantum transition experiment (INADEQUATE) method. Exemplary 2D NMR methods include correlation spectroscopy (COSY), total correlation spectroscopy (TOCSY), 2D-INADEQUATE, 2D-adequate double quantum transfer experiment (ADEQUATE), nuclear overhauser effect spectroscopy (NOSEY), rotating-frame NOE spectroscopy (ROESY), heteronuclear multiple-quantum correlation spectroscopy (HMQC), heteronuclear single quantum coherence spectroscopy (HSQC), short range coupling and long range coupling methods. Exemplary solid state NMR method include solid state ¹³Carbon NMR, high resolution magic angle spinning (HR-MAS) and cross polarization magic angle spinning (CP-MAS) NMR methods. Exemplary NMR techniques include diffusion ordered spectroscopy (DOSY), DOSY-TOCSY and DOSY-HSQC.

In some embodiments, the results from the mass spectroscopy method are analyzed by an algorithm for protein identification. In some embodiments, the algorithm combines the results from the mass spectroscopy method with a protein sequence database for protein identification. In some embodiments, the algorithm comprises ProLuCID algorithm, Probity, Scaffold, SEQUEST, or Mascot.

In some embodiments, a value is assigned to each of the protein from the probe-protein complex. In some embodiments, the value assigned to each of the protein from the probe-protein complex is obtained from the mass spectroscopy analysis. In some instances, the value is the area-under-the curve from a plot of signal intensity as a function of mass-to-charge ratio. In some instances, the value correlates with the reactivity of a Lys residue within a protein.

In some instances, a ratio between a first value obtained from a first protein sample and a second value obtained from a second protein sample is calculated. In some instances, the ratio is greater than 2.5, 3, 3.5, 4, 4.5, 5, 6, 7, 8, 9, 10, 11, 12, 13, 14, 15, 16, 17, 18, 19, or 20. In some cases, the ratio is at most 20.

In some instances, the ratio is calculated based on averaged values. In some instances, the averaged value is an average of at least two, three, or four values of the protein from each cell solution, or that the protein is observed at least two, three, or four times in each cell solution and a value is assigned to each observed time. In some instances, the ratio further has a standard deviation of less than 12, 10, or 8.

In some instances, a value is not an averaged value. In some instances, the ratio is calculated based on value of a protein observed only once in a cell population. In some instances, the ratio is assigned with a value of 20.

In some instances the compounds and methods are disclosed in “Function-first proteomic strategies for chemical probe discovery in human cells”, Lazear, M. R. et al.; which is herein incorporated by reference in its entirety.

### Kits

Disclosed herein, in certain embodiments, are kits and articles of manufacture for use to generate a target protein-ligand adduct or with one or more methods described herein. In some embodiments, described herein is a kit for detecting a target protein-ligand interaction. In some embodiments, the kit includes small molecule ligands described herein, small molecule fragments or libraries, and/or controls, and reagents suitable for carrying out one or more of the methods described herein. In some instances, the kit further comprises samples, such as a cell sample, and suitable solutions such as buffers or media. In some embodiments, the kit further comprises recombinant target protein for use in one or more of the methods described herein. In some embodiments, additional components of the kit comprises a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use.

For example, the container(s) include probes, test compounds, and one or more reagents for use in a method disclosed herein. Such kits optionally include an identifying description or label or instructions relating to its use in the methods described herein.

A kit may include labels listing contents and/or instructions for use, and package inserts with instructions for use. A set of instructions may be included.

In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 15% of that number. The term “about” a range refers to that range minus 15% of its lowest value and plus 15% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

“Carboxyl” refers to —COOH.

“Cyano” refers to —CN.

“Alkyl” refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₆ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term —C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₈ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₅ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈alkynylene). In other embodiments, an alkynylene comprises three to five carbon atoms (i.e., C₃-C₅ alkynylene).

“Aryl” refers to a radical derived from a hydrocarbon ring system comprising at least one aromatic ring. In some embodiments, an aryl comprises hydrogens and 6 to 30 carbon atoms. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂.

“Aralkyl” refers to a radical of the formula —R^{c}-aryl where R^{c} is an alkylene chain as defined above, for example, methylene, ethylene, and the like.

“Aralkenyl” refers to a radical of the formula —R^{d}-aryl where R^{d} is an alkenylene chain as defined above. “Aralkynyl” refers to a radical of the formula —R^{c}-aryl, where R^{c} is an alkynylene chain as defined above.

“Carbocycle” refers to a saturated, unsaturated or aromatic rings in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom), bridged, or spiro ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₅ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2,2,2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Cycloalkylalkyl” refers to a radical of the formula —R^{c}-cycloalkyl where R^{c} is an alkylene chain as described above.

“Cycloalkylalkoxy” refers to a radical bonded through an oxygen atom of the formula —O—R^{c}-cycloalkyl where R^{c} is an alkylene chain as described above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro,2-fluoroethane.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heterocycle” refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. “Heterocyclene” refers to a divalent heterocycle linking the rest of the molecule to a radical group.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, phosphorous, and sulfur. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized.

Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₅ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3- to 6-membered heterocycloalkyl. In some embodiments, the cycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to, the monosaccharides, the disaccharides, and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroaryl” or “aromatic heterocycle” refers to a ring system radical comprising carbon atom(s) and one or more ring heteroatoms selected from the group consisting of nitrogen, oxygen, phosphorous, silicon, and sulfur, and at least one aromatic ring. In some embodiments, a heteroaryl is a 5- to 14-membered ring system radical comprising one to thirteen carbon atoms, one to six heteroatoms selected from the group consisting of nitrogen, oxygen, phosphorous, and sulfur. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). For example, a 5-membered heteroaryl ring or 5-membered aromatic heterocycle has 5 endocyclic atoms, e.g., triazole, oxazole, thiophene, etc. Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The section headings used herein are for organizational purposes only and are not to be construed as limiting the subject matter described.

### EXAMPLES

The following examples are included for illustrative purposes only and are not intended to limit the scope of the invention.

### Example 1: Function-First Proteomic Strategies for Chemical Probe Discovery in Human Cells

Most proteins in the human proteome lack chemical probes, and several large-scale and generalizable small-molecule binding assays have been introduced to address this problem. How compounds discovered in such “binding-first” assays affect protein function, however, often remains unclear. Described here are proteomic strategies to assess the global impact of compounds on multiple functional features of proteins in cells, including biomolecular interactions, expression, and stability. Integrating these “function-first” readouts with cysteine reactivity profiling discriminates changes in protein complexation state and turnover that are caused by site-specific liganding events, including the stereoselective engagement of cysteines in PSME1 and SF3B1 that cause disassembly of the PA28 proteasome regulatory complex and remodeling of the spliceosome, respectively. The findings thus show how multidimensional proteomic analysis of focused libraries of compounds can expedite the discovery of chemical probes with site-specific functional effects on proteins in human cells.

### INTRODUCTION

Chemical probes are useful tools for perturbing proteins and pathways in biological systems and can serve as starting points for novel therapeutics. The discovery of chemical probes has historically relied on the advent of specific functional assays for proteins of interest and devising such assays can present a major technical hurdle, especially for proteins that lack readily monitorable biochemical activities. Efforts to expand the druggable proteome have accordingly begun to introduce complementary approaches for ligand discovery that leverage binding assays with near-universal applicability to diverse types of proteins. Examples of technologies for the discovery of small-molecule binders of proteins include fragment-based screening, DNA-encoded libraries (DELs), and chemical proteomics. These methods have illuminated the broad small-molecule binding potential of proteins from structurally and functionally distinct classes. Nonetheless, whether and how small molecule-protein interactions emanating from binding-first assays may impact the functions of proteins in biological systems remain open and important questions, ones that are particularly challenging to address on a global scale when confronted with the divergent, specialized activities performed by proteins in the cell.

In considering ways to relate small-molecule binding events to functional outcomes, an emerging category of phenotypic assays that provide broad biochemical signatures of cell states was looked to. The impact of compounds on global gene and protein expression profiles of cells has, for instance, been assessed using DNA microarrays and mass spectrometry (MS)-based proteomics, respectively. These past studies have, however, mostly evaluated compounds with known mechanisms of action, where the gene/protein expression profiles have served to augment understanding of established functional effects on proteins or to reveal potential off-target toxicities. When applied to naïve compounds that lack complementary protein-binding profiles, the problem of relating biochemical signatures to specific protein targets persists. Of interest here was to determine whether the integration of two types of global profiling data—biochemical signatures and protein-binding—could facilitate the de novo discovery of chemical probes that produce functional outcomes in human cells.

Because small molecules can perturb other biochemical features beyond gene/protein expression, the following was developed: a complementary “function-first” signatures of compound action, specifically, global readouts of protein complexation states in cells. Many proteins function as parts of larger homo- or hetero-typic complexes, and small-molecule inhibitors or stabilizers of protein-protein interactions (PPIs) can serve as valuable chemical probes and therapeutic agents. Several approaches have been introduced for the large-scale mapping of PPIs, including genetic (e.g., yeast-two hybrid) and biochemical (affinity purification or co-fractionation coupled with MS) methods. Among these options, co-fractionation-MS was considered compatible with devising a streamlined and minimally biased platform for monitoring the effects of small molecules on PPIs in human cells.

Presented here is a proteomic workflow that integrates function-first assays of electrophilic compound effects on both the i) complexation state and ii) expression of proteins with iii) global and site-specific maps of covalent protein binding. From a modest number of compounds (<20), diverse types of chemical probes were discovered that, for instance, react site-specifically with the adaptor protein PSME1 to disrupt the proteasome activator complex PA28, and the splicing factor SF3B1 to functionally remodel the spliceosome. Further shown is how the integration of functional and binding data acquired at a proteome-wide scale can facilitate the discrimination of compounds that promote protein degradation through single-versus multi-site liganding mechanisms. Taken together, the findings provide a roadmap for chemical probe discovery that emphasizes the attributes of covalent chemistry and stereochemistry coupled with global proteomic readouts of small-molecule binding and the functional properties of proteins.

### Results

### A Proteomic Platform to Discover Small-Molecule Modulators of Protein-Protein Interactions (PPIs)

Chemical probes targeting PPIs can be challenging to discover from conventional compound libraries due the extensive points of contact sometimes required to perturb large protein interfaces. This problem has been addressed by structure-guided approaches that facilitate the linking of weakly binding fragments into higher-affinity compounds capable of blocking PPIs. Covalent compounds may offer an alternative and possibly more ligand-efficient strategy, where the permanent bonds formed with proteins may be sufficient to disrupt PPIs even at single points of contact. Studies have shown that electrophilic small molecule-sensitive, or ligandable, cysteines, are found on a wide array of proteins from different structural and functional classes, indicating that diverse types of protein complexes may be sensitive to electrophilic compound action.

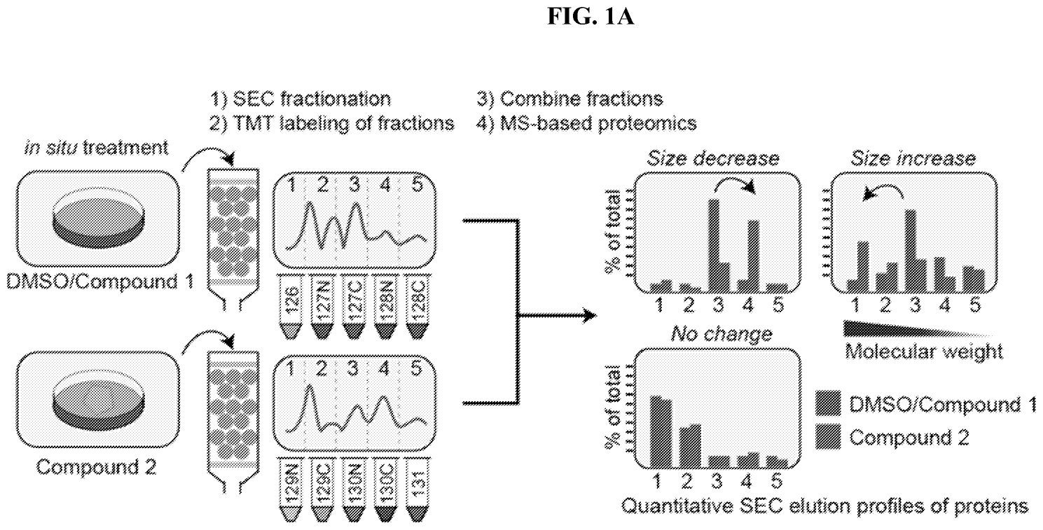

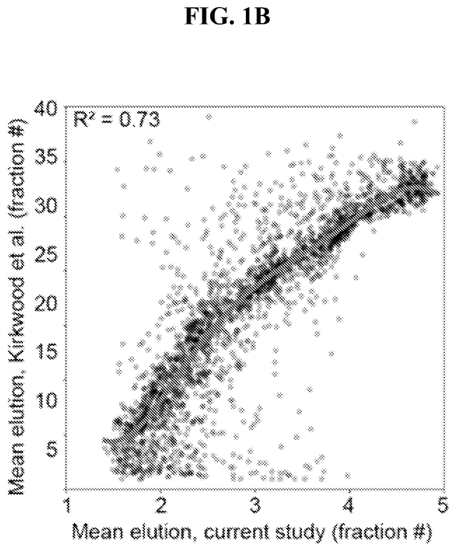

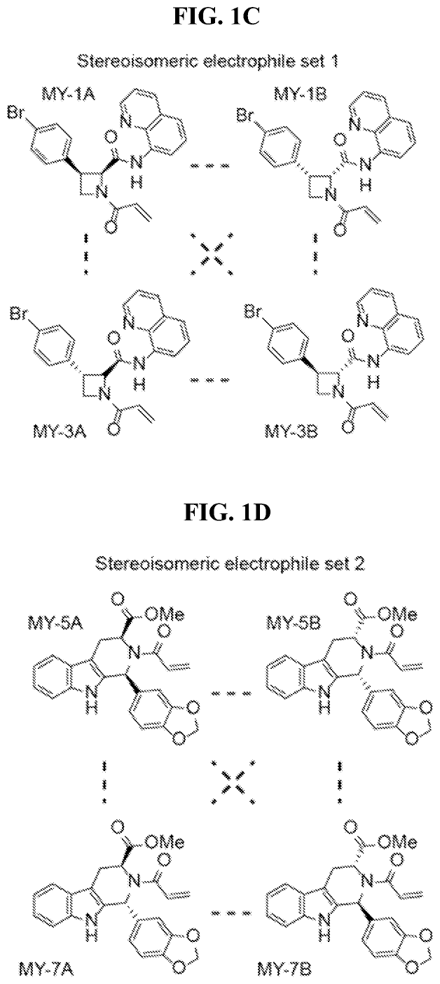

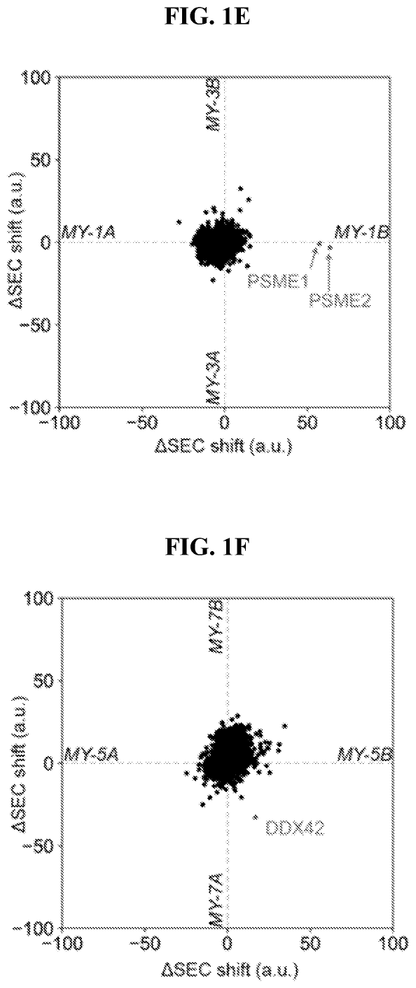

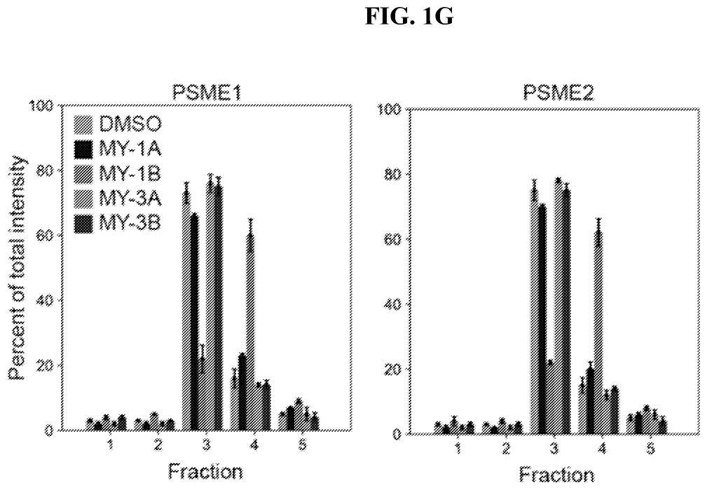

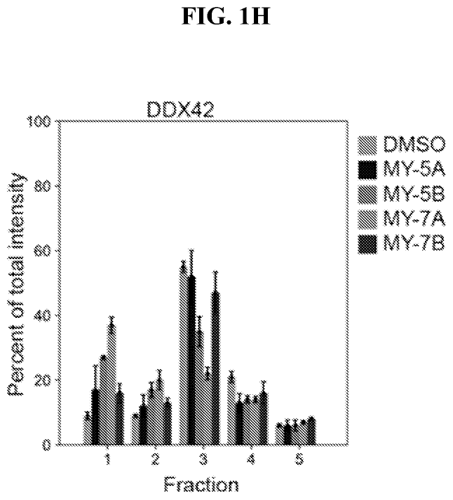

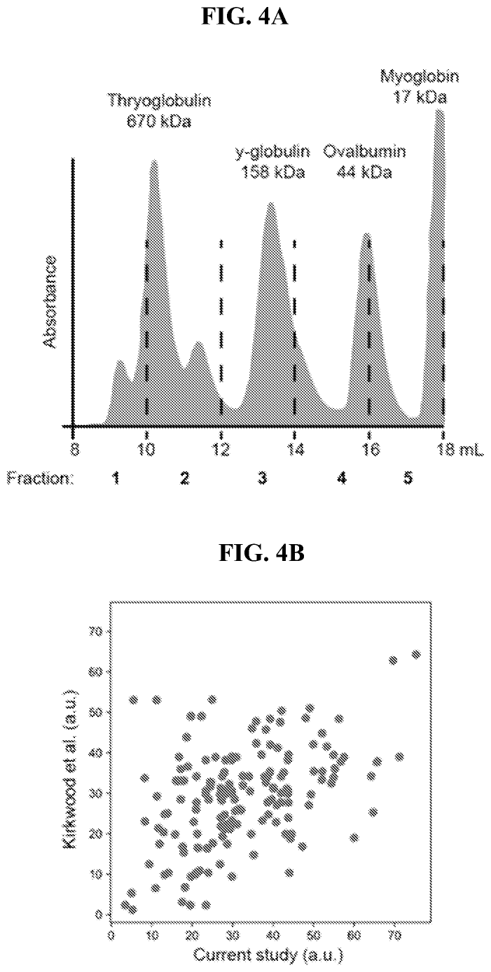

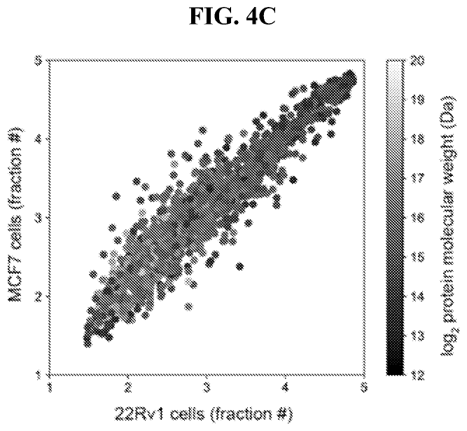

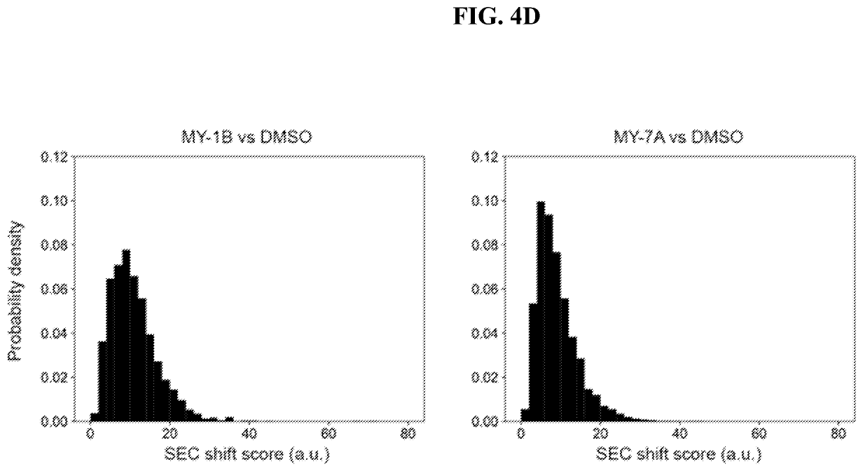

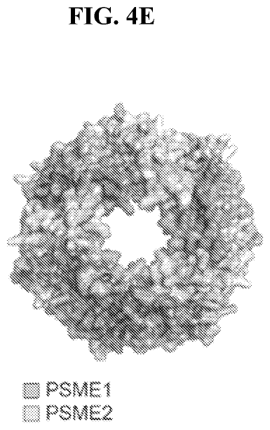

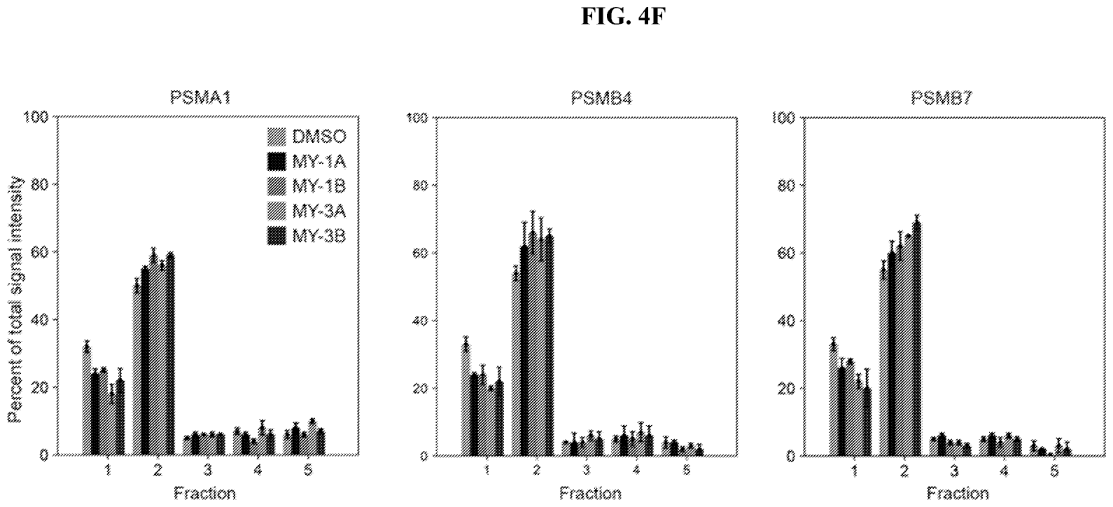

To more broadly explore the potential of covalent compounds to alter PPIs, a size exclusion chromatography (SEC)/MS-based proteomic method was developed that compares the migration profiles of proteins from human cells treated with different cysteine-directed electrophilic small molecules. Similar co-fractionation-MS, or protein correlation profiling, studies using sophisticated SEC protocols have been introduced to construct large-scale de novo protein-protein interaction networks. A more simplified variant of this approach with fewer fractionation steps and less MS instrument time may be useful for identifying proteins undergoing substantial shifts in SEC migration following exposure of human cells to electrophilic compounds. FIGS. 1A-1H and 4A-4F demonstrate a proteomic platform to discover small-molecule modulators of protein-protein interactions in human cells. A protocol was established to compare five SEC fractions from two cell-treatment conditions in a single tandem mass tagging (TMT)-based proteomic analysis (FIG. 1A). Proteins quantified from soluble proteomic lysates of the human prostate cancer cell line 22Rv1 collected from SEC experiments performed with a Superdex 200 Increase 10/300GL column spanned a wide range of molecular weights (FIG. 4A) and showed good correlation with data from previous co-fractionation MS experiments using a much larger number of SEC fractions (40 fractions) (FIG. 1B). Likewise, the majority of protein complexes (as defined by the CORUM Core Complex database) for which two or more protein members were identified in our experiments and previous SEC-MS studies displayed similar co-elution scores (FIG. 4B). Mean elution times for proteins were consistent both within replicate SEC-MS experiments performed on the same human cancer cell line and across experiments performed on distinct human cancer cell lines (FIG. 4C). On average, each SEC-MS experiment quantified ˜3700 total proteins (minimum of two unique quantified peptides per protein) from a starting material of 1.25 mg of soluble human cancer cell proteome. Taken together, these data indicate that a five-fraction SEC-MS protocol exhibited sufficient resolution and sensitivity to evaluate the effects of electrophilic compounds on a diverse array of protein complexes in human cells.

22Rv1 cells were exposed to two structurally distinct sets of four stereoisomeric electrophilic compounds (FIGS. 1C-1D; 20 μM, 3 h treatment), which were constructed based on principles of diversity-oriented synthesis (DOS), specifically the use of sp3-rich, entropically constrained, densely functionalized, and stereochemically defined scaffolds. One set of compounds representing tryptoline acrylamides stereoselectively engaged cysteine residues on diverse classes of proteins in human T cells. Accordingly, a goal here was to identify proteins that showed compound-induced stereoselective shifts in their SEC migration profiles (size shifts), which could then be correlated with stereoselective effects of the compounds on cysteine reactivity. Stereoselective size shifts, or ‘shift scores’, were quantified by calculating the Euclidean distance between individual protein elution profiles across the five SEC fractions collected from cells treated with enantiomeric compound pairs. An additional comparison to DMSO-treated cells enabled determination of which of the two enantiomeric compounds caused the observed shift(s) in protein migration. The vast majority of protein migration profiles were not stereoselectively altered in cells treated with enantiomeric pairs of electrophilic compounds (FIG. 1E-1F, 4D). However, discrete and striking stereoselective changes in the migration profiles of individual proteins were observed in cells treated with the azetidine acrylamide MY-1B (1) (FIG. 1E) and the tryptoline acrylamide MY-7A (7) (FIG. 1F).

MY-1B treatment caused a decreased size shift for two proteins—PSME1 and PSME2—which both moved from a higher molecular weight (MW) fraction 3 to a lower MW fraction 4 (FIG. 1G). None of the other azetidine acrylamide stereoisomers (enantiomer MY-1A and diastereomers MY-3A and MY-3B) affected the SEC profiles of PSME1 and PSME2 (FIGS. 1E and 1G). PSME1 and PSME2 form the heptameric PA28 proteasomal regulatory complex (FIG. 4E), which regulates antigenic peptide processing by the immunoproteasome. The coordinated MY-1B-induced shifts in SEC profiles for PSME1 and PSME2 indicated that MY-1B may disrupt the PA28 complex. In contrast, subunits of the 20S core proteasomal complex were unaffected by MY-1B treatment and generally migrated, as expected, in a higher MW fraction (FIG. 4F). The major stereoselective effect of MY-7A was an increased size shift for the RNA helicase DDX42 from a lower MW fraction 3 to a higher MW fraction 1 (FIGS. 1F and 1H). The enantiomeric compound MY-7B did not affect DDX42, while a more modest, but observable stereoselective shift for this protein was observed with MY-5B compared to MY-5A (FIGS. 1F and 1H). These data suggested that MY-7A, and to a lesser extent MY-5B, may promote the assembly of DDX42 into a larger protein complex.

### MY-1B Disrupts the PA28 Complex by Engaging C22 of PSME1

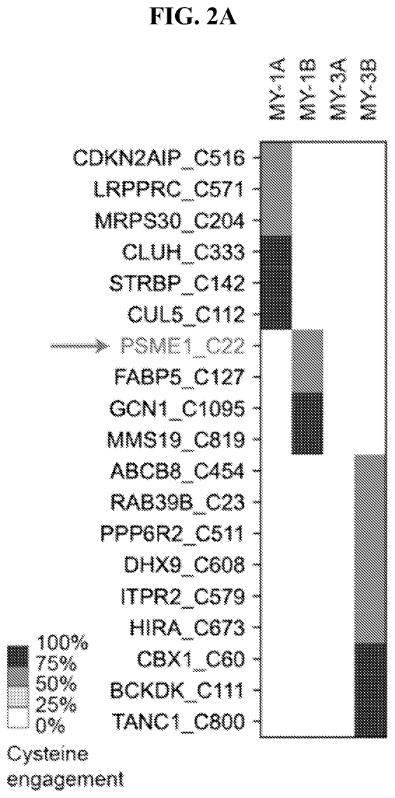

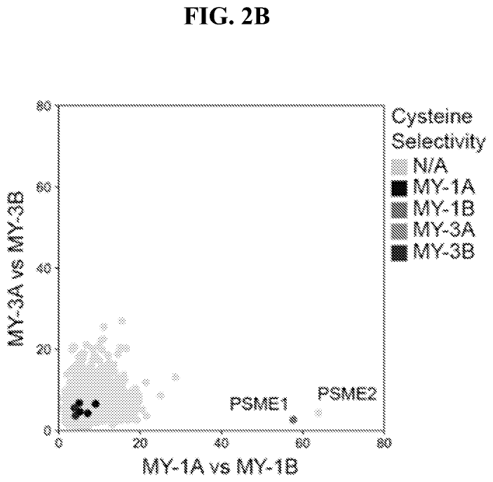

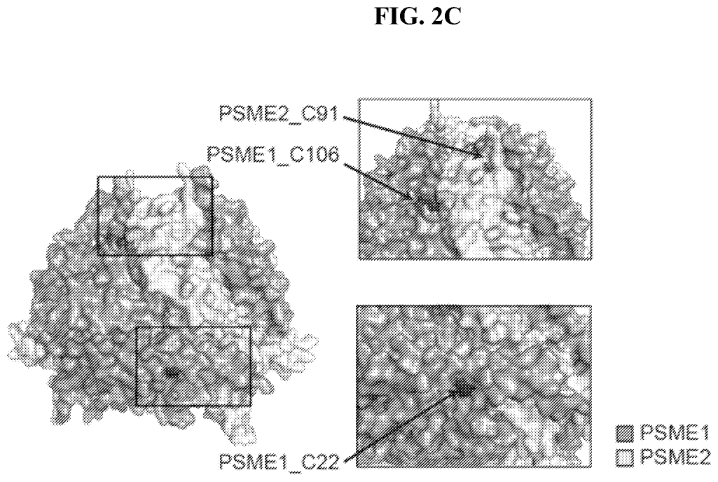

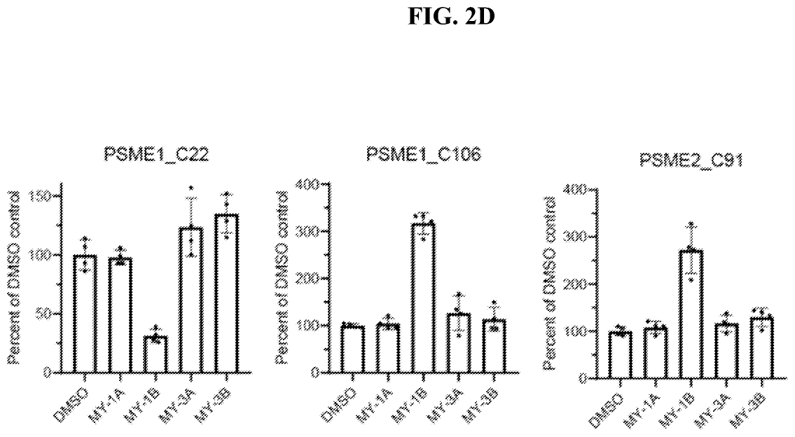

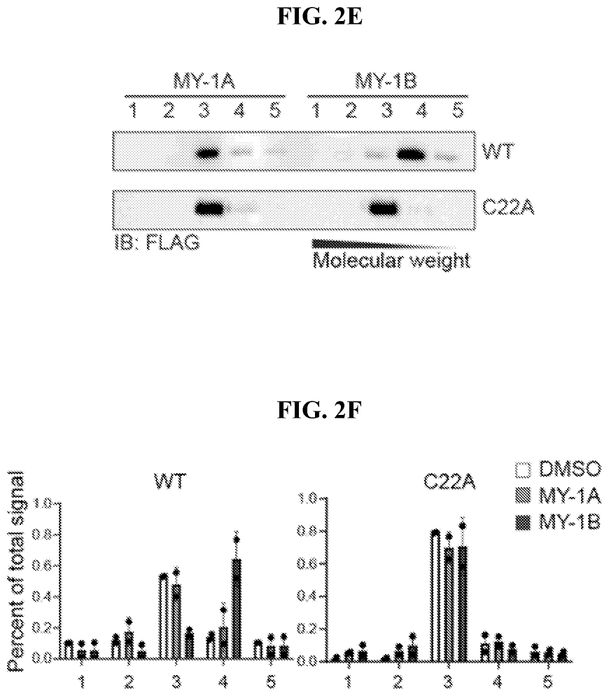

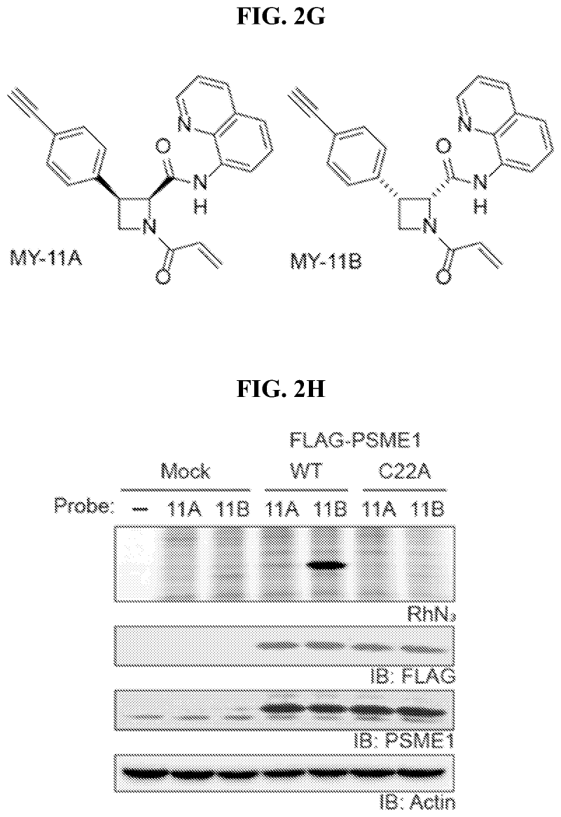

To determine the mechanistic basis for the changes in protein migration caused by electrophilic compounds, a chemical proteomic analysis was performed of cysteines that were stereoselectively engaged by these compounds in 22Rv1 cells. In these experiments, proteomes from DMSO- or compound-treated cells were exposed to the broad-spectrum cysteine-reactive probe iodoacetamide-desthiobiotin (IA-DTB), and probe-labeled cysteines enriched and quantified by multiplexed (TMT) MS analysis. Cysteines of interest were designated as those showing a substantial loss (>66%) in IA-DTB labeling in cells treated with the active acrylamide compared to its inactive stereoisomers. FIG. 2A-2H show that electrophilic compounds stereoselectively disrupt the PA28 complex by engaging C22 on PSME1. Of the ˜20,000 total quantified cysteines, a handful were stereoselectively engaged by MY-1B, including C22 of PSME1 (FIG. 2A-2B ant Table 1), a residue that is located near the PSME1-PSME2 interface (FIG. 2C). Data in Table 1 include stereoselective delta-shift values (a.u.). Several other cysteines in PSME1 and PMSE2 were quantified, but none showed loss in signal in MY-1B-treated cells (FIG. 2D). On the contrary, one additional cysteine in PSME1 (C106), as well as one cysteine in PSME2 (C91), exhibited striking, stereoselective increases in reactivity in MY-1B-treated cells (FIG. 2D). Interestingly, PSME1_C106 and PSME2_C91 are also located proximal to the protein-protein interface of PSME1 and PSME2 (FIG. 2C), suggesting that these residues may become more solvent accessible following PSME1_C22 engagement by MY-1B and providing further evidence that this compound causes a substantial alteration in the structure of the PA28 complex.

TABLE 1
Cysteine engagement for proteins engaged (>66%)
by stereoisomer probe set 1 in 22Rv1 cells

| | MY-1A | MY-1B | MY-3A | MY-3B |
|---|---|---|---|---|
| | (% | (% | (% | (% |
| | engage- | engage- | engage- | engage- |
| site | ment) | ment) | ment) | ment) |
| CUL5_C112 | 95.5 | 11 | −14.75 | −20 |
| STRBP_C142 | 88.25 | 8.5 | 16.25 | 7.75 |
| CLUH_C333 | 81 | 13 | 0.5 | −4.5 |
| MRPS30_C204 | 74.5 | −37 | 1 | −41.5 |
| LRPPRC_C571 | 73.5 | 7 | 20 | 5 |
| CDKN2AIP_C516 | 66.5 | 24 | 12.75 | 18.75 |
| MMS19_C819 | 1.5 | 90 | −10 | 11 |
| GCN1_C1095 | 18.5 | 84 | 5 | −7.5 |
| FABP5_C127 | 0 | 73 | −13 | −60.5 |
| PSME1_C22 | −2.25 | 67.75 | −14.5 | −31.75 |
| TANC1_C800 | −2 | 3 | 12.5 | 83.5 |
| BCKDK_C111 | 17.25 | 14.5 | 19.5 | 76.75 |
| CBX1_C60 | −11.5 | −14.5 | 14.5 | 75 |
| HIRA_C673 | −12 | 3 | 11.5 | 73 |
| ITPR2_C579 | 7.75 | 11.25 | 4.5 | 70.75 |
| DHX9_C608 | −10.5 | −11 | 16 | 69.5 |
| PPP6R2_C511 | 9.5 | 10 | 15.5 | 68.5 |
| ABCB8_C454 | −1.25 | 14 | 24 | 67 |
| RAB39B_C23 | 24.5 | 18.5 | −53 | 67 |

Recombinant PSME1 forms homooligomeric structures in the absence of PSME2, providing a convenient assay to further explore the effects of electrophilic compounds on PSME1-dependent PPIs. Recombinantly expressed, FLAG epitope-tagged WT-PSME1 or a C22A mutant of this protein exhibited similar SEC elution profiles to endogenous PSME1 with predominant migration in fraction 3 (FIG. 2E). Treatment of cells with MY-1B, but not MY-1A caused a clear size shift for recombinant WT-PMSE1 to fraction 4, while the C22A mutant was unaffected (FIG. 2E-2F). These data thus support that MY-1B disrupts PSME1-mediated PPIs by specifically engaging C22. Also confirmed was site-specific and stereoselective engagement of PSME1_C22 by an alkynylated analogue of MY-1B (MY-11B, FIG. 2G) combined with copper-catalyzed azide-alkyne cycloaddition (CuAAC or click) chemistry conjugation to an azide-rhodamine reporter tag (FIG. 2H).

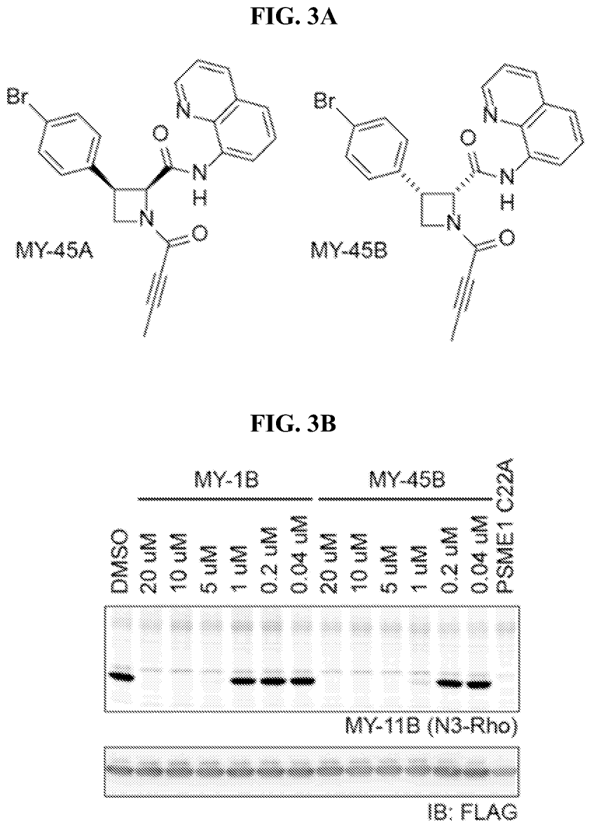

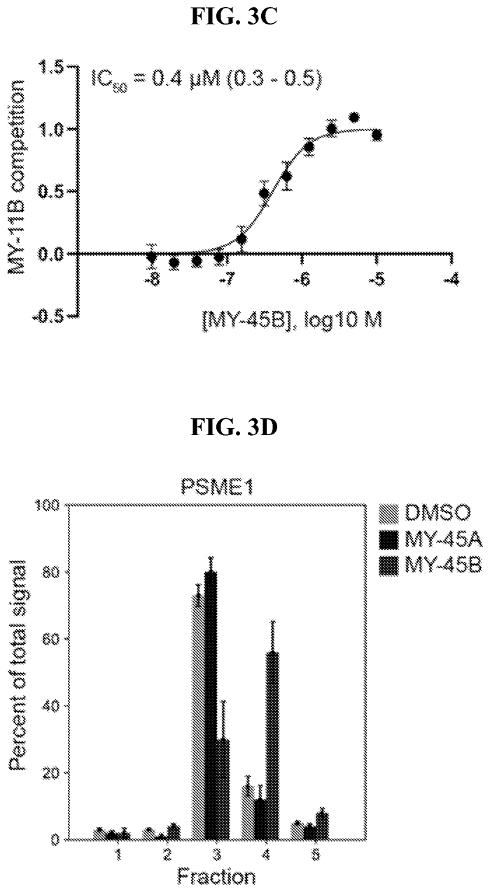

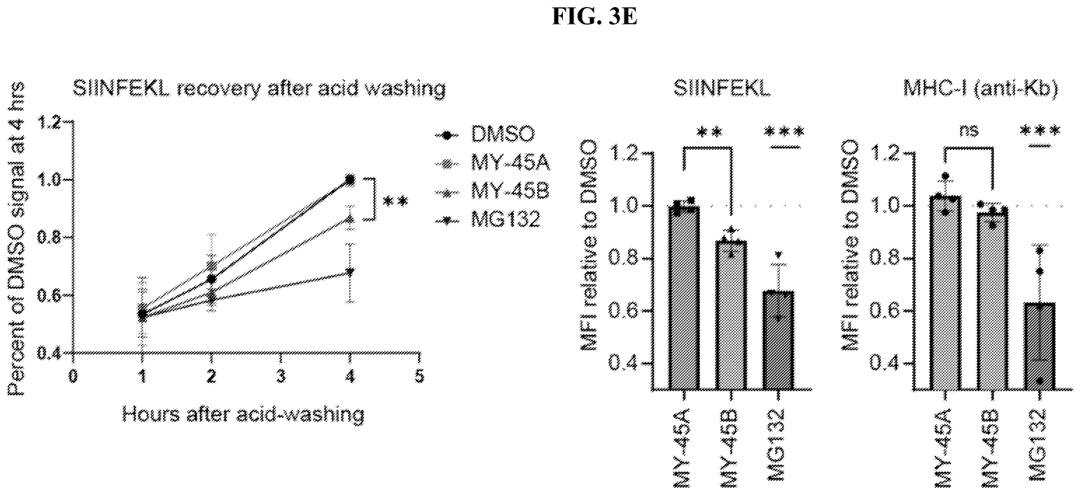

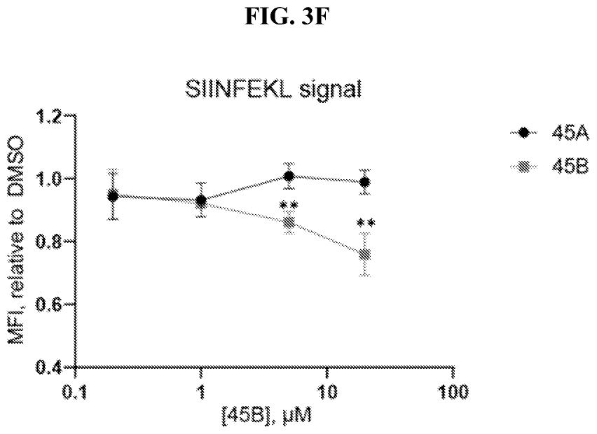

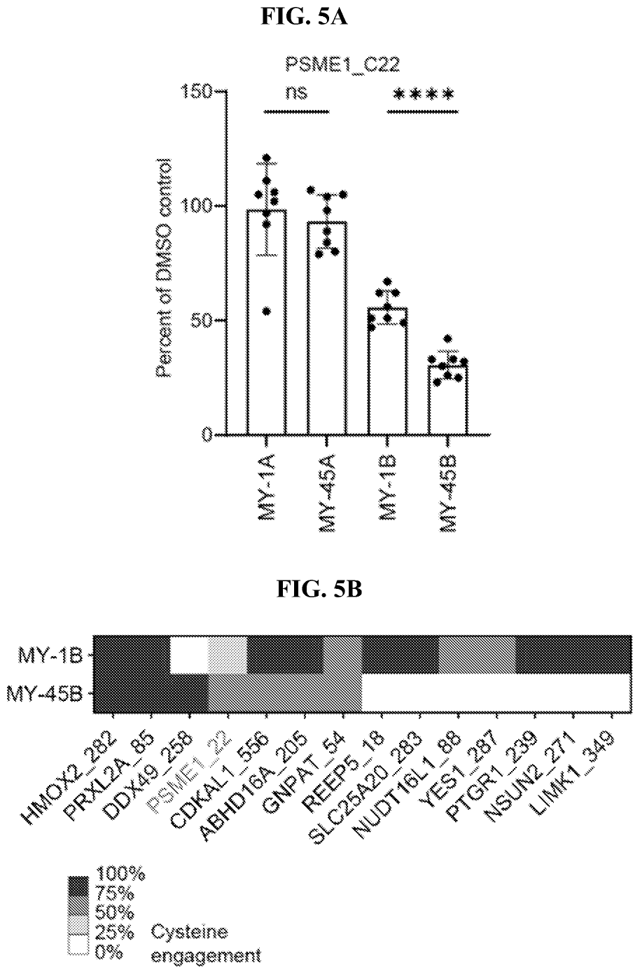

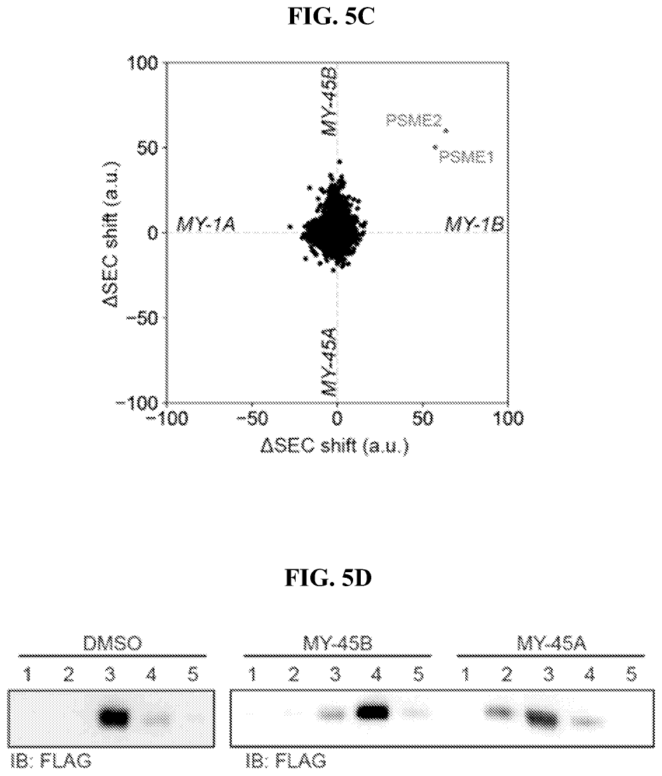

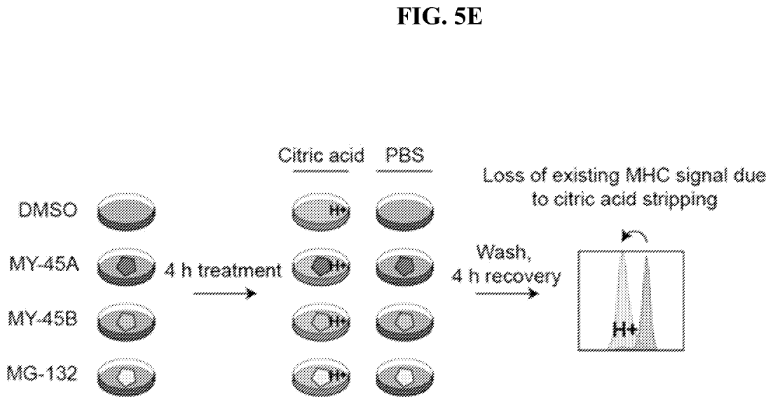

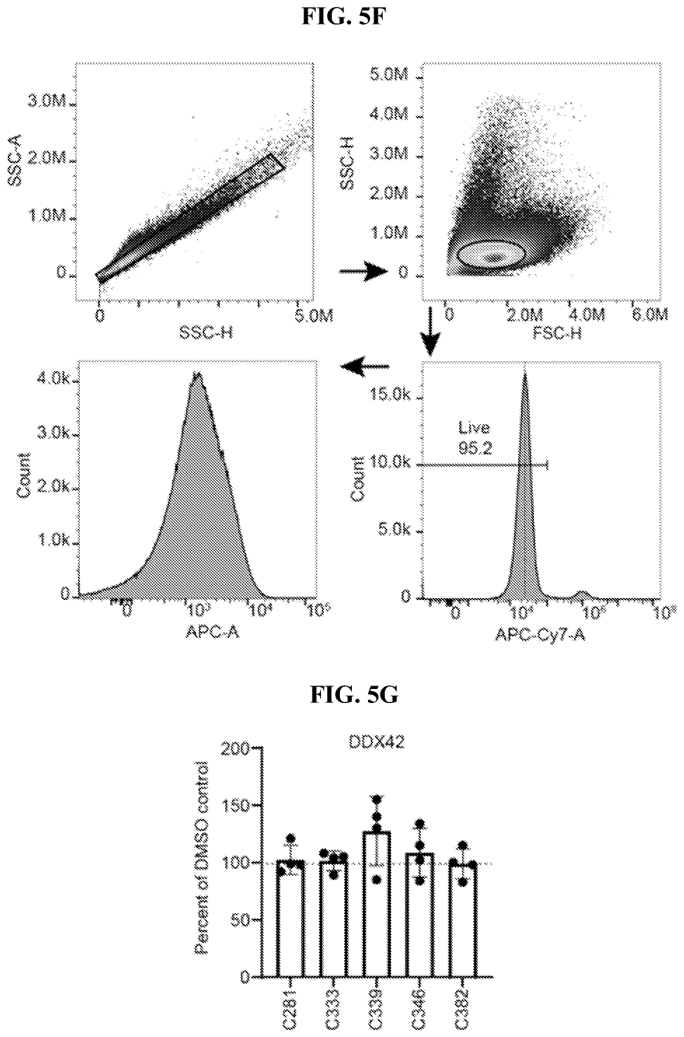

Substituting an acrylamide with a less reactive butynamide electrophile can improve selectivity for certain protein targets like the B-cell kinase BTK. The butynamide analogue of MY-1B-MY-45B (FIG. 3A)—showed even greater potency for engaging PSME1_C22 (FIG. 3B), displaying an IC50 value of ˜0.4 μM (FIG. 3C). FIG. 5A-5G shows that electrophilic compounds stereoselectively disrupt the structure and function of the PA28 complex by engaging C22 of PSME1. It was confirmed by cysteine reactivity profiling that MY-45B stereoselectively engaged C22 of endogenous PSME1 in 22Rv1 cells (FIG. 5A and Table 2) with improved potency and selectivity compared to MY-1B (FIG. 5B). Data in Table 2 include percent engagement relative to DMSO (higher is more engaged). Across >15,000 quantified cysteines, MY-45B stereoselectively engaged a single additional cysteine in 22Rv1 cells—C₂₅₈ of the helicase DDX49 (FIG. 5B)—but this protein has not been implicated in proteasome regulation. MY-45B, but not its inactive enantiomer MY-45A (FIG. 2A), caused the expected SEC migration shifts for endogenous PSME1 and PSME2 (FIGS. 3D and 5C), as well as recombinant PSME1 (FIG. 5D and Table 3). Data in Table 3 include percent engagement relative to DMSO (higher is more engaged). The overall properties of MY-45B, including good potency and proteome-wide selectivity, as well as pairing with an inactive enantiomer, led to the designation of this compound as a suitable chemical probe for studying the function of PSME1 and the PA28 complex.

TABLE 2
Cysteine engagement for proteins engaged (>66%) by stereoisomer probe set 1 in 22Rv1 cells

| | | MY-1A (% | MY-1B (% | MY-3A (% | MY-3B (% | | max (% |
|---|---|---|---|---|---|---|---|
| | | engage- | engage- | engage- | engage- | Stereo- | engage- |
| site | accession | ment) | ment) | ment) | ment) | selective | ment) |
| CDKN2AIP_C516 | Q9NXV6 | 66.5 | 24 | 12.75 | 18.75 | MY-1A | 66.5 |
| CLUH_C333 | O75153 | 81 | 13 | 0.5 | −4.5 | MY-1A | 81 |
| CUL5_C112 | Q93034 | 95.5 | 11 | −14.75 | −20 | MY-1A | 95.5 |
| LRPPRC_C571 | P42704 | 73.5 | 7 | 20 | 5 | MY-1A | 73.5 |
| MRPS30_C204 | Q9NP92 | 74.5 | −37 | 1 | −41.5 | MY-1A | 74.5 |
| STRBP_C142 | Q96SI9 | 88.25 | 8.5 | 16.25 | 7.75 | MY-1A | 88.25 |
| FABP5_C127 | Q01469 | 0 | 73 | −13 | −60.5 | MY-1B | 73 |
| GCN1_C1095 | Q92616 | 18.5 | 84 | 5 | −7.5 | MY-1B | 84 |
| MMS19_C819 | Q96T76 | 1.5 | 90 | −10 | 11 | MY-1B | 90 |
| PSME1_C22 | Q06323 | −2.25 | 67.75 | −14.5 | −31.75 | MY-1B | 67.75 |
| ABCB8_C454 | Q9NUT2 | −1.25 | 14 | 24 | 67 | MY-3B | 67 |
| BCKDK_C111 | O14874 | 17.25 | 14.5 | 19.5 | 76.75 | MY-3B | 76.75 |
| CBX1_C60 | P83916 | −11.5 | −14.5 | 14.5 | 75 | MY-3B | 75 |
| DHX9_C608 | Q08211 | −10.5 | −11 | 16 | 69.5 | MY-3B | 69.5 |
| HIRA_C673 | P54198 | −12 | 3 | 11.5 | 73 | MY-3B | 73 |
| ITPR2_C579 | Q14571 | 7.75 | 11.25 | 4.5 | 70.75 | MY-3B | 70.75 |
| PPP6R2_C511 | O75170 | 9.5 | 10 | 15.5 | 68.5 | MY-3B | 68.5 |
| RAB39B_C23 | Q96DA2 | 24.5 | 18.5 | −53 | 67 | MY-3B | 67 |
| TANC1_C800 | Q9C0D5 | −2 | 3 | 12.5 | 83.5 | MY-3B | 83.5 |
| ABCA2_C2385 | Q9BZC7 | 33 | 78 | 42.5 | 72.5 | none | 78 |
| ABCB8_C461 | Q9NUT2 | −3 | 0 | 32.5 | 66.5 | none | 66.5 |
| ABCB8_C462 | Q9NUT2 | 28.5 | 39 | 19.5 | 73.25 | none | 73.25 |
| ABHD16A_C205 | O95870 | 67.75 | 80.25 | 73.25 | 77.25 | none | 80.25 |
| ACTL8_C197 | Q9H568 | 14 | 66.25 | 3.25 | 36.75 | none | 66.25 |
| ALB_C269 | P02768 | 42.5 | 47.5 | 86 | 79.5 | none | 86 |
| ALG3_C21 | Q92685 | 90.25 | 68 | 74.5 | 84.75 | none | 90.25 |
| ANGEL1_C27 | Q9UNK9 | 63 | 66.5 | 45.5 | 54.5 | none | 66.5 |
| ANKRD46_C61 | Q86W74 | 17.5 | 28.5 | 5.5 | 66.5 | none | 66.5 |
| APOOL_C74 | Q6UXV4 | 52.75 | 64.5 | 52 | 71.25 | none | 71.25 |
| APOOL_C79 | Q6UXV4 | 54.25 | 73 | 48 | 69 | none | 73 |
| ATP9A_C31 | O75110 | 84.5 | 79.5 | 73 | 86.5 | none | 86.5 |
| BCKDK_C118 | O14874 | 55.5 | 57.75 | 43 | 78.5 | none | 78.5 |
| BLOC1S2_C41 | Q6QNY1 | 6 | 70 | 3.5 | 66.5 | none | 70 |
| C3orf33_C275 | Q6P1S2 | 83.5 | 83 | 85 | 84 | none | 85 |
| CCNE2_C109 | O96020 | 77 | 76.75 | 21.75 | 42.75 | none | 77 |
| CDKAL1_C556 | Q5VV42 | 59.5 | 91 | 88.5 | 89 | none | 91 |
| CENPC_C291 | Q03188 | −1.5 | 7 | 28 | 82.5 | none | 82.5 |
| CEP128_C915 | Q6ZU80 | −13 | 67.5 | 7 | 25 | none | 67.5 |
| CHUK_C406 | O15111 | 51.5 | 28.5 | 0 | 97 | none | 97 |
| CKAP4_C100 | Q07065 | 84 | 90.25 | 78.75 | 93.75 | none | 93.75 |
| CNOT4_C175 | O95628 | 0 | −15 | 76.5 | 76 | none | 76.5 |
| COX6B1_C54 | P14854 | 66.5 | 56 | 33.5 | 36.5 | none | 66.5 |
| CPT1A_C96 | P50416 | 87 | 84.75 | 89.25 | 93.5 | none | 93.5 |
| CRYBG1_C1574 | Q9Y4K1 | 23 | 72 | 28.5 | 13.5 | none | 72 |
| CTC1_C584 | Q2NKJ3 | 20.25 | 37.25 | 23.5 | 66.25 | none | 66.25 |
| CYP27B1_C163 | O15528 | 73 | 89 | 73.5 | 100 | none | 100 |
| DCAF1_C1113 | Q9Y4B6 | −18 | 68.5 | −1.5 | 26.5 | none | 68.5 |
| DCTN4_C258 | Q9UJW0 | 27.5 | 68 | 15.5 | 31.5 | none | 68 |
| DCUN1D4_C219 | Q92564 | 45.5 | 82.5 | 33.5 | −17 | none | 82.5 |
| DDX39A_C86 | O00148 | 44.5 | 37 | 23.5 | 67.5 | none | 67.5 |
| DDX39B_C87 | Q13838 | 44.5 | 37 | 23.5 | 67.5 | none | 67.5 |
| DENND2B_C804 | P78524 | 29.25 | 80.25 | −5.25 | −5.75 | none | 80.25 |
| DHRS13_C174 | Q6UX07 | 60.25 | 69.75 | 42 | 61.75 | none | 69.75 |
| EPB42_C586 | P16452 | 55 | 33.5 | 66.5 | 43.5 | none | 66.5 |
| EPHX4_C287 | Q8IUS5 | 23.5 | 44.5 | 28 | 86.5 | none | 86.5 |
| ESRP1_C551 | Q6NXG1 | 51 | 73.5 | 6.5 | 69 | none | 73.5 |
| FXYD3_C63 | Q14802 | 48.5 | 52.5 | 43.5 | 71 | none | 71 |
| GNPAT_C122 | O15228 | 70.5 | 74.5 | 35.5 | 51.5 | none | 74.5 |
| GNPAT_C54 | O15228 | 88.5 | 91.5 | 54.25 | 77.5 | none | 91.5 |
| GPAT3_C306 | Q53EU6 | 75.75 | 81.5 | 57 | 100 | none | 100 |
| GPAT4_C325 | Q86UL3 | 83.25 | 80 | 67.25 | 93.75 | none | 93.75 |
| GRIN2C_C1105 | Q14957 | 78.5 | 40.5 | 85 | 64.5 | none | 85 |
| GSTO1_C32 | P78417 | 22 | 82 | 79 | 80.5 | none | 82 |
| HARS2_C173 | P49590 | 84.75 | 63.75 | 35.25 | 35.75 | none | 84.75 |
| HARS2_C175 | P49590 | 100 | 67 | 11.5 | 44.5 | none | 100 |
| HMOX2_C265 | P30519 | 72.5 | 73.5 | 71.75 | 79.5 | none | 79.5 |
| HMOX2_C282 | P30519 | 93.5 | 96.25 | 96.25 | 97.75 | none | 97.75 |
| HNRNPLL_C235 | Q8WVV9 | 15 | 34 | −7.5 | 75 | none | 75 |
| HSD11B2_C264 | P80365 | 30.5 | 22 | 23.5 | 90.25 | none | 90.25 |
| HSDL1_C265 | Q3SXM5 | 52 | 54 | 64.25 | 82 | none | 82 |
| IFT43_C58 | Q96FT9 | 28.5 | −17 | 17 | 71.5 | none | 71.5 |
| ITPR2_C292 | Q14571 | 24.5 | 70 | 48.5 | 50.5 | none | 70 |
| KRTCAP3_C197 | Q53RY4 | 61 | 69.5 | 31 | 54 | none | 69.5 |
| LAPTM4A_C20 | Q15012 | 69 | 55 | 49.5 | 70.5 | none | 70.5 |
| LAPTM4A_C23 | Q15012 | 67 | 54.5 | 48 | 72 | none | 72 |
| LIMK1_C349 | P53667 | 45.25 | 93 | 21.5 | 43.25 | none | 93 |
| LMF2_C659 | Q9BU23 | 66 | 65.75 | 44.75 | 69.25 | none | 69.25 |
| LMNA_C570 | P02545 | 75 | 70.5 | 75.5 | 70.5 | none | 75.5 |
| MAGI1_C526 | Q96QZ7 | 65 | 32 | 2.5 | 100 | none | 100 |
| MGST3_C56 | O14880 | 58.75 | 58 | 63.5 | 73.5 | none | 73.5 |
| MTMR12_C152 | Q9C0I1 | 40.5 | 31 | 12 | 91.5 | none | 91.5 |
| NAPIL1_C132 | P55209 | −21.5 | 6 | 25 | 80 | none | 80 |
| NCAPD2_C439 | Q15021 | 29.5 | 20 | 21.5 | 80 | none | 80 |
| NELFCD_C169 | Q8IXH7 | 51 | 41.75 | 40.75 | 69.5 | none | 69.5 |
| NSD2_C1018 | O96028 | 16.5 | 43.75 | 36.75 | 67 | none | 67 |
| NSUN2_C271 | Q08J23 | 42.25 | 88.75 | −8.25 | −11 | none | 88.75 |
| NSUN3_C298 | Q9H649 | 35.5 | 55.5 | 38 | 83 | none | 83 |
| NT5DC1_C119 | Q5TFE4 | 49 | 63.25 | 93.5 | 79.5 | none | 93.5 |
| NT5DC2_C504 | Q9H857 | 85.5 | 75 | 54.5 | 59 | none | 85.5 |
| NT5DC2_C507 | Q9H857 | 83.75 | 67.25 | 50.5 | 57.5 | none | 83.75 |
| NUDT16L1_C88 | Q9BRJ7 | 33.25 | 88.5 | 14.5 | 87.5 | none | 88.5 |
| NUDT8_C207 | Q8WV74 | 71.5 | 78.5 | 64.25 | 88.75 | none | 88.75 |
| NUP205_C224 | Q92621 | 28.5 | 6 | 100 | 30.5 | none | 100 |
| OCIAD1_C98 | Q9NX40 | 26 | 19 | 12.5 | 72.5 | none | 72.5 |
| PAFAH2_C72 | Q99487 | 61.75 | 61 | 56.75 | 81 | none | 81 |
| PDE3B_C311 | Q13370 | 57.5 | 58 | 49 | 75.5 | none | 75.5 |
| PDP1_C149 | Q9P0J1 | 67 | 37.25 | 19.25 | 37.25 | none | 67 |
| PLPPR2_C207 | Q96GM1 | 30.5 | 31 | 46.5 | 71 | none | 71 |
| PNPLA8_C100 | Q9NP80 | 67 | 59.5 | 48 | 70.5 | none | 70.5 |
| PRAF2_C34 | O60831 | 76 | 74 | 55.5 | 78.5 | none | 78.5 |
| PRORP_C507 | O15091 | 6.75 | 74.25 | 1.25 | 34.5 | none | 74.25 |
| PRRT3_C720 | Q5FWE3 | 47 | 53 | 45.5 | 71 | none | 71 |
| PRXL2A_C85 | Q9BRX8 | 92.5 | 98.75 | 97.5 | 98.5 | none | 98.75 |
| PRXL2A_C88 | Q9BRX8 | 96.25 | 97.25 | 95 | 97.25 | none | 97.25 |
| PTGES2_C110 | Q9H7Z7 | 31.25 | 38.5 | 69.75 | 89.25 | none | 89.25 |
| PTGR1_C239 | Q14914 | 75 | 79 | −4 | −16.5 | none | 79 |
| RAB40B_C77 | Q12829 | 100 | 100 | −39 | 12.5 | none | 100 |
| REEP5_C18 | Q00765 | 91.75 | 97.5 | 88.25 | 91.75 | none | 97.5 |
| RETSAT_C547 | Q6NUM9 | 80 | 66.75 | 53.5 | 71.25 | none | 80 |
| RTN4_C1101 | Q9NQC3 | 85 | 87.5 | 91.5 | 85 | none | 91.5 |
| SCAMP4_C20 | Q969E2 | 80 | 85.5 | 72.5 | 100 | none | 100 |
| SLC12A8_C660 | A0AV02 | 77.5 | 61.5 | 41 | 68.5 | none | 77.5 |
| SLC25A16_C311 | P16260 | 79 | 37.5 | 32 | 38.5 | none | 79 |
| SLC25A20_C283 | O43772 | 37 | 92 | 66 | −241 | none | 92 |
| SLC25A20_C89 | O43772 | 9.5 | 94 | 45 | 91 | none | 94 |
| SLC25A39_C334 | Q9BZJ4 | 23 | 45.5 | 30.5 | 72 | none | 72 |
| SNX11_C181 | Q9Y5W9 | 54.5 | 68.5 | 17 | 23 | none | 68.5 |
| SPATA2_C41 | Q9UM82 | 58 | 91.75 | 24.5 | 29.5 | none | 91.75 |
| SRC_C280 | P12931 | 17.75 | 92.25 | 2 | 64 | none | 92.25 |
| STK11_C418 | Q15831 | 83.5 | 66.5 | 38 | 61.5 | none | 83.5 |
| SUPT3H_C268 | O75486 | 27 | 34.5 | 62.5 | 70.5 | none | 70.5 |
| SYNE2_C2993 | Q8WXH0 | −20 | 76.5 | 26.5 | 54.5 | none | 76.5 |
| SYNE2_C2994 | Q8WXH0 | 29.5 | 75.75 | 11.25 | 37.5 | none | 75.75 |
| TBC1D13_C282 | Q9NVG8 | 75.75 | 1 | −16 | 51.5 | none | 75.75 |
| TM7SF2_C184 | O76062 | 86.5 | 64 | 66.5 | 100 | none | 100 |
| TMEM256- | I3L3X5 | 81 | 86 | 58 | 66 | none | 86 |
| PLSCR3_C126 | | | | | | | |
| TMEM50A_C13 | O95807 | 63.5 | 58 | 60.5 | 68.5 | none | 68.5 |
| TOMM70_C214 | O94826 | 7.5 | 29 | 30.5 | 71.5 | none | 71.5 |
| TRMT61A_C209 | Q96FX7 | 40.75 | 67 | 18.75 | 56 | none | 67 |
| TRPM4_C758 | Q8TD43 | 40.5 | 54.5 | 29.5 | 73.5 | none | 73.5 |
| TRPM4_C759 | Q8TD43 | 69.75 | 48.75 | 50.25 | 76.25 | none | 76.25 |
| TTC3_C109 | P53804 | 80.5 | 54.5 | 4.5 | 21.5 | none | 80.5 |
| UBR5_C2094 | O95071 | 23 | 40.5 | 22.5 | 81 | none | 81 |
| UNC13A_C999 | Q9UPW8 | 54.5 | 100 | 100 | 23.5 | none | 100 |
| VPS18_C522 | Q9P253 | −22 | 45 | 88 | 21 | none | 88 |
| VWA5B2_C1040 | Q8N398 | 76.5 | 76.25 | 51.5 | 74.25 | none | 76.5 |
| WAPL_C1133 | Q7Z5K2 | 18 | 34 | 78.5 | 46 | none | 78.5 |
| WDR45B_C63 | Q5MNZ6 | 52.25 | 7.25 | 0 | 86.75 | none | 86.75 |
| XXYLT1_C10 | Q8NBI6 | 76.5 | 77 | 67 | 93 | none | 93 |
| YES1_C287 | P07947 | 5.75 | 94.5 | 0.5 | 42.25 | none | 94.5 |
| ZBED4_C599 | O75132 | 16 | 39 | 21 | 83.5 | none | 83.5 |
| ZFAT_C392 | Q9P243 | 32.5 | 39 | 17.5 | 68.5 | none | 68.5 |
| ZFC3H1_C1778 | O60293 | 70.75 | 7 | 47.75 | −22.75 | none | 70.75 |
| ZNF346_C68 | Q9UL40 | 95.5 | 37 | 14 | 45.25 | none | 95.5 |

TABLE 3
Cysteine engagement for proteins engaged
(>66%) by MY-1B or 45B in 22Rv1 cells

| | MY-1B (% | 45B (% |
|---|---|---|
| site | engagement) | engagement) |
| HMOX2_282 | 94.00 | 92.13 |
| PRXL2A_85 | 91.48 | 86.23 |
| DDX49_258 | 5.50 | 82.25 |
| PSME1_22 | 44.38 | 69.50 |
| CDKAL1_556 | 79.33 | 67.00 |
| ABHD16A_205 | 83.67 | 62.50 |
| GNPAT_54 | 73.17 | 51.83 |
| REEP5_18 | 79.00 | 22.75 |
| SLC25A20_283 | 82.75 | 20.50 |
| NUDT16L1_88 | 67.63 | 9.38 |
| YES1_287 | 67.13 | 5.50 |
| PTGR1_239 | 81.33 | −4.50 |
| NSUN2_271 | 94.50 | −13.13 |
| LIMK1_349 | 90.88 | −15.75 |

The PA28 complex may impact MHC class I (MHC-I) presentation through influencing the proteasomal processing of a select, but not exhaustively determined, set of antigens. The chicken ovalbumin peptide SIINFEKL may be used as a model peptide in antigen presentation studies and is among the antigens regulated by the PA28 complex. Using a kinetic assay that measures the rate of recovery of MHC-I peptide presentation following acid washing (FIGS. 5E and 2F), mouse T lymphoma cells constitutively expressing chicken ovalbumin (E.G7-Ova) showed a time- and concentration-dependent reduction in SIINFEKL presentation—but not overall MHC-I presentation—following treatment with MY-45B, but not MY-45A (FIGS. 3E and 3F). In contrast, the direct proteasome inhibitor MG132 suppressed both SIINFEKL peptide and MHC-I presentation (FIG. 3E). These data, taken together, indicate that chemical probes targeting PSME1_C22 may disrupt both the structure and function of the PA28 proteasome regulatory complex.

Also chemical proteomic experiments were performed with the tryptoline acrylamides, but MY-7A-sensitive cysteines in DDX42 were not detected (FIG. 5G). The chemical proteomic studies more broadly revealed several other proteins harboring cysteines that were stereoselectively engaged by electrophilic compounds, but did not show alterations in their migration in SEC-MS experiments (FIG. 2B). These results emphasize the value of a function-first assay such as SEC-MS that can illuminate discrete covalent liganding events affecting the complexation state of proteins.

### Protein Abundance Changes Caused by Electrophilic Compounds and Stereoselective Covalent Modulators of the Spliceosome

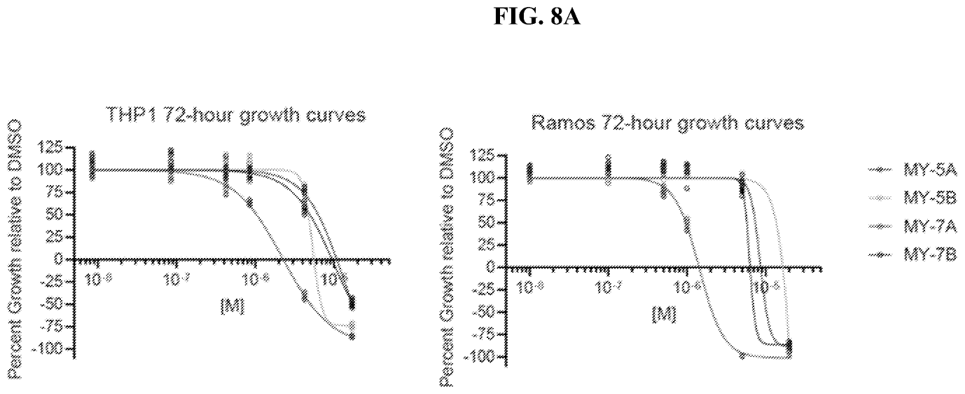

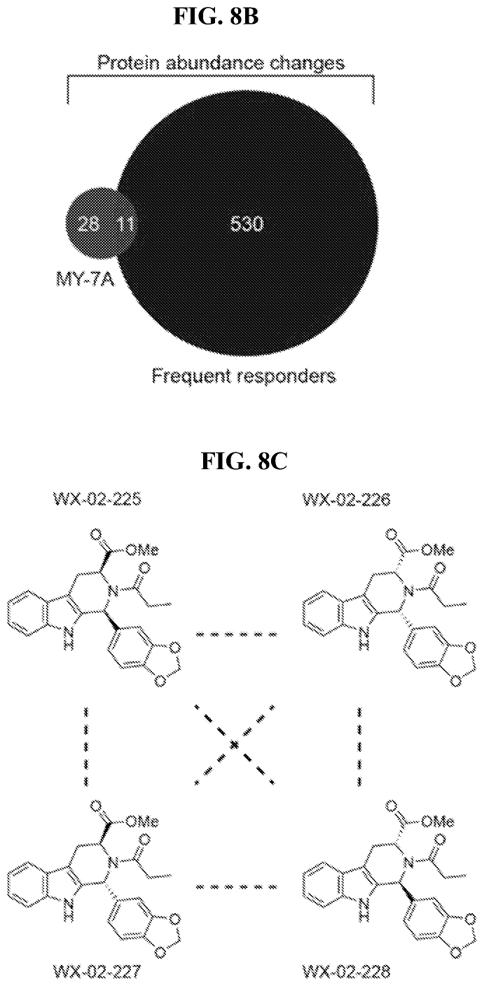

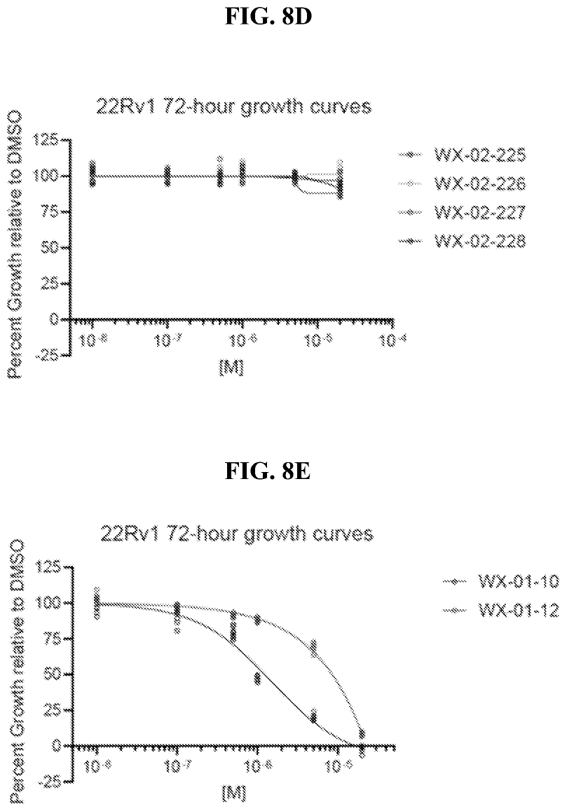

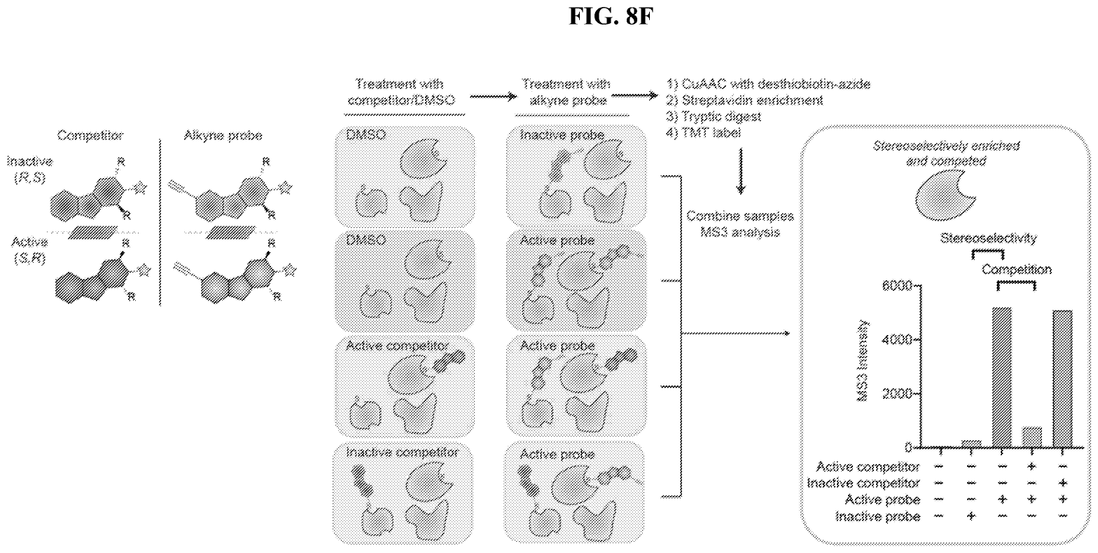

FIG. 6A-6G indicate that electrophilic compounds that stereoselectively engage SF3B1 altered protein expression profiles of cancer cells and blocked their proliferation. Protein abundance profiles of cancer cells treated with stereoisomeric electrophilic compound sets were evaluated, and tryptoline acrylamide MY-7A caused a striking, stereoselective reduction in several proteins within 8 h post-treatment in 22Rv1 cells (FIG. 6A). Surprisingly, however, none of the affected proteins appeared to possess MY-7A-sensitive cysteines, which argued against a direct, ligand-induced degradation model. Gene Ontology (GO)-term analysis revealed that the MY-7A-sensitive proteins were enriched in cell division and cell cycle functions (FIG. 6B), suggesting that these protein changes may be an indirect consequence of MY-7A impacting the proliferation of cancer cells. Consistent with this hypothesis, MY-7A caused a stereoselective blockade in the growth of 22Rv1 cells (FIG. 6C) and other human cancer cell lines (FIG. 8A). Interestingly, however, the MY-7A-induced protein changes showed only modest overlap with a “frequent responder” group of proteins that may represent common changes caused by diverse cytotoxic compounds (FIG. 8B), suggesting a discrete mechanism of anti-proliferative action for MY-7A. FIG. 8A-8F indicate that electrophilic compounds that stereoselectively engage SF3B1 may alter the protein expression profiles and block the proliferation of cancer cells.

These data, taken together, reveal how a function-first approach that measures protein abundance changes in cancer cells can identify electrophilic compounds that promote the direct degradation of protein targets by both single- and multi-site modification, as well as alter the expression of proteins that mark an apparently specific form of cancer cell growth blockade. The various underlying mechanisms appear to reflect intrinsic features of the protein targets, as it was observed that similar electrophilic compound-induced protein changes in different cancer cell lines.

Attention was next turned to elucidating the mechanistic basis for the stereoselective anti-proliferative effects caused by MY-7A. Recognizing that some electrophilic compound-sensitive cysteines may evade detection by cysteine reactivity profiling, if, for instance, they reside on non-proteotypic peptides (i.e., tryptic peptides that are infrequently detected by MS-based proteomics due to size and/or poor physicochemical properties), a chemical proteomic strategy was adopted to identify stereoselectively engaged targets of MY-7A. First, alkyne-modified analogues of MY-7A and its inactive enantiomer MY-7B—WX-01-10 and WX-01-12, respectively, were generated (FIG. 6D)—and it was confirmed that these compounds maintained differential cell growth inhibitory effects (FIG. 8E). Concurrently found, through screening a larger set of tryptoline acrylamides, was a morpholino amide analogue of MY-7A-WX-02-23—that exhibited ˜4-fold greater antiproliferative activity (IC50 of 170 nM (150-180 nM)) while maintaining excellent stereoselectivity in comparison to the enantiomeric compound WX-02-43 (FIGS. 6D and 6E). With this set of chemical tools, protein targets were pursued by pre-treating cancer cells with WX-02-23 or its inactive enantiomer WX-02-43, followed by treatment with active alkyne WX-01-10 or inactive alkyne WX-01-12, where a protein target responsible for the observed cytotoxicity effect should display: (i) stereoselective enrichment by WX-01-10 (in comparison to WX-01-12) as read out by CuAAC chemical proteomics; and (ii) competition in its enrichment by WX-02-23, but not WX-02-43 (FIG. 8F). Only a single protein—the spliceosome factor SF3B1—was found to be meet the aforementioned criteria (FIGS. 6F and 6G).

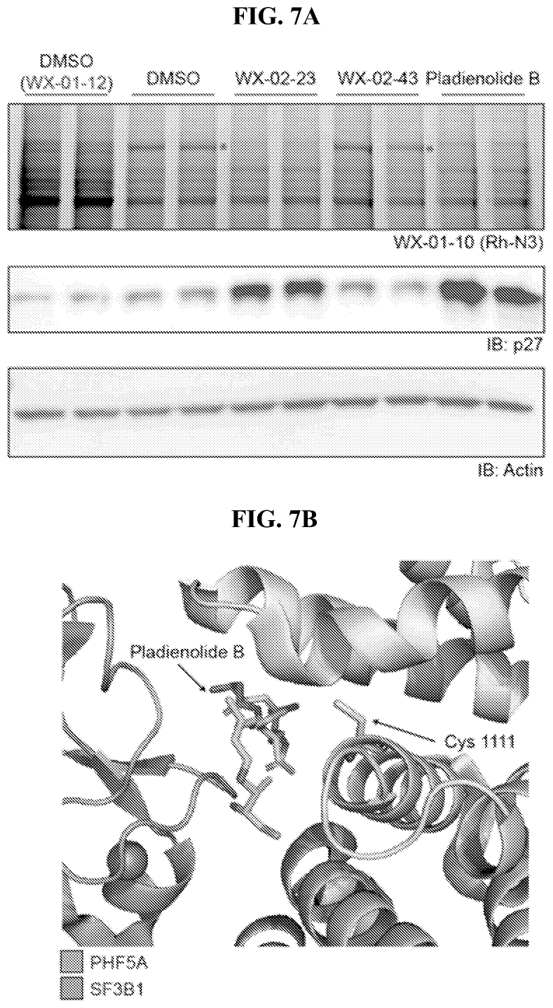

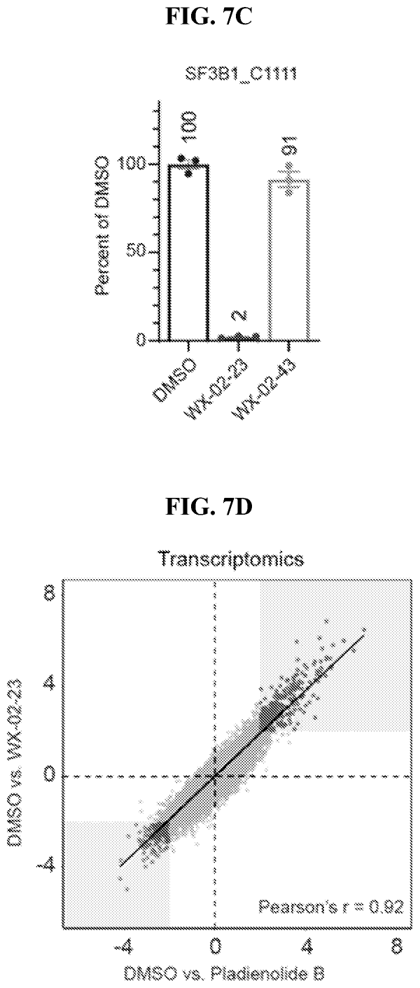

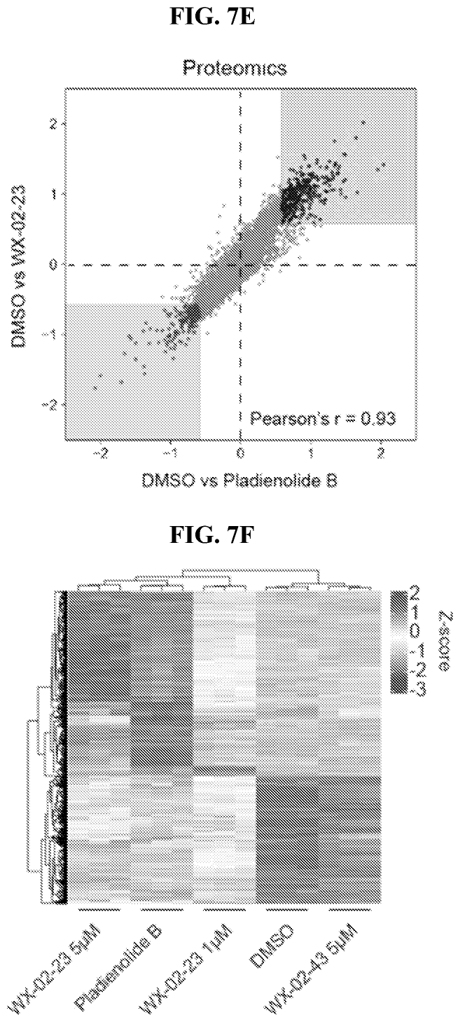

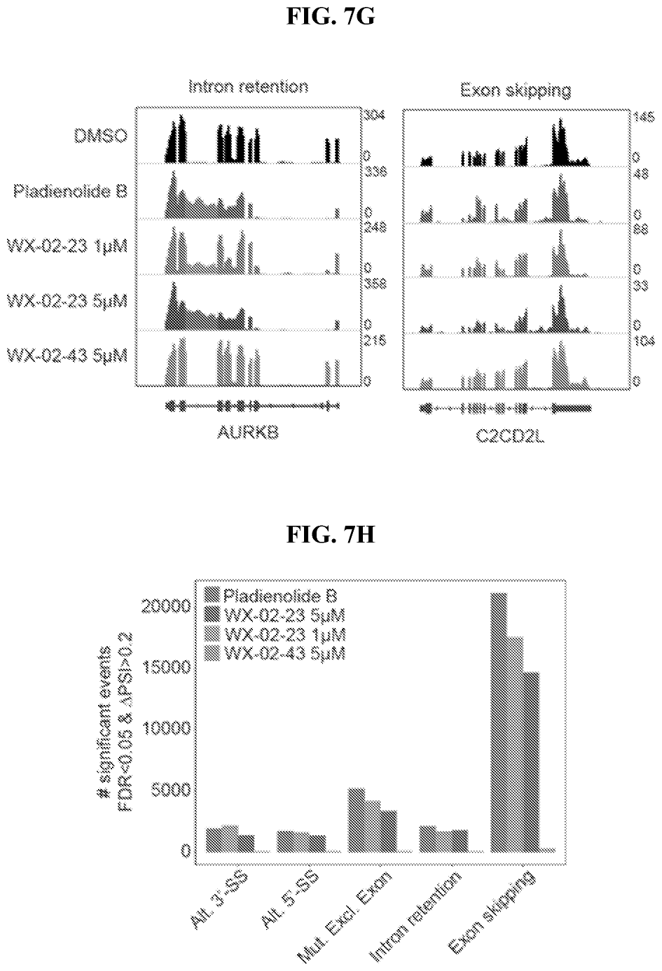

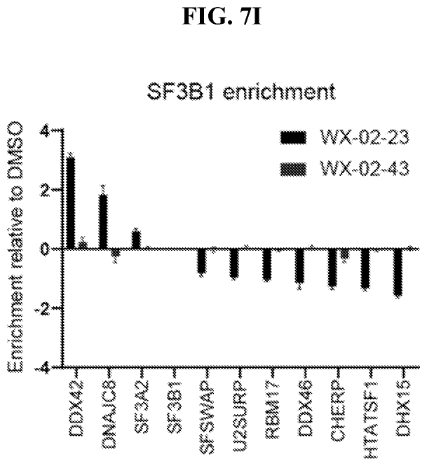

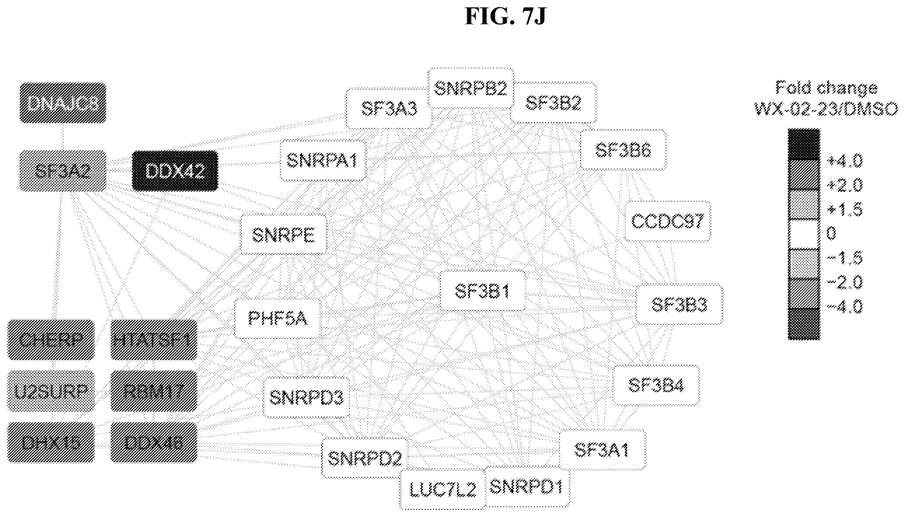

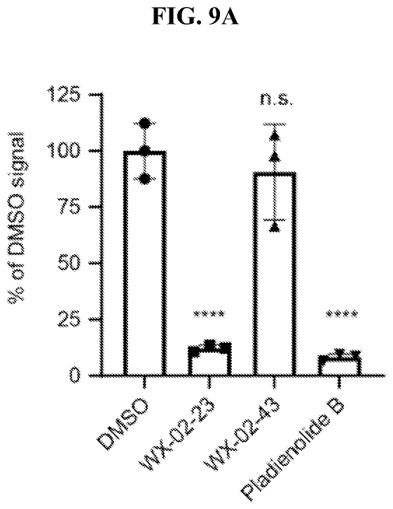

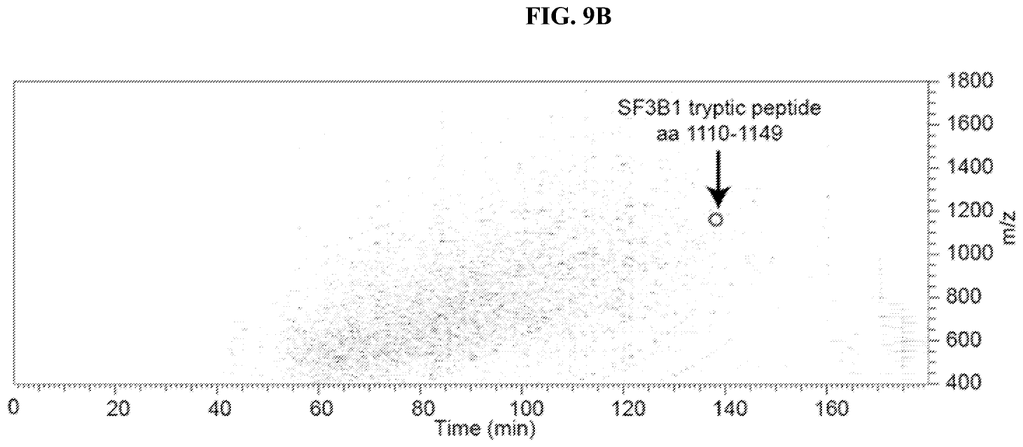

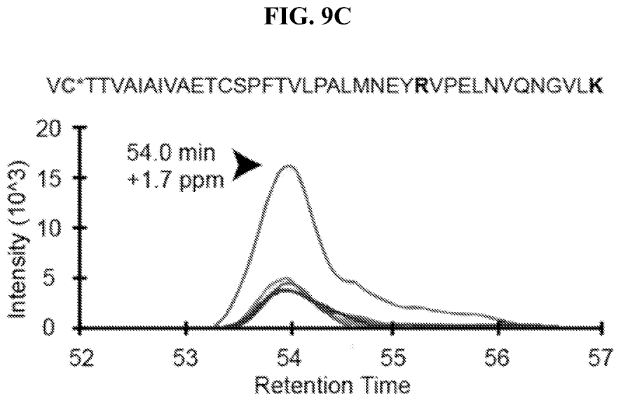

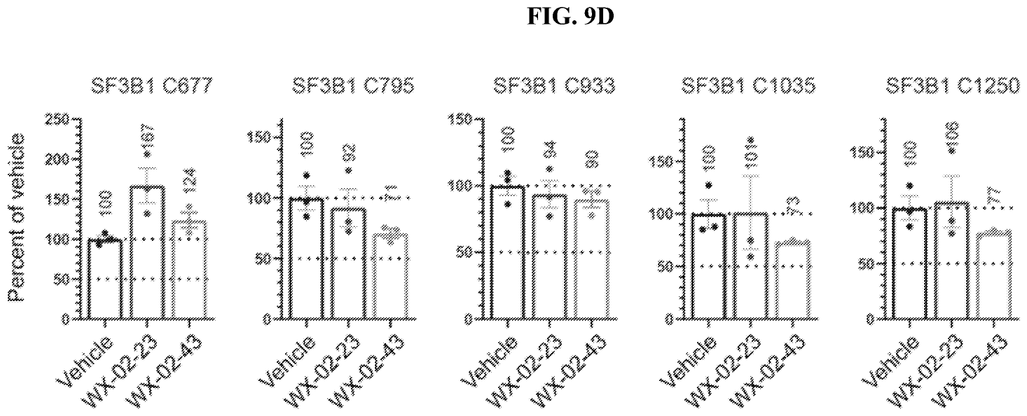

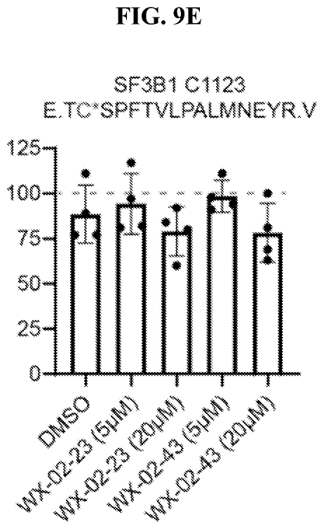

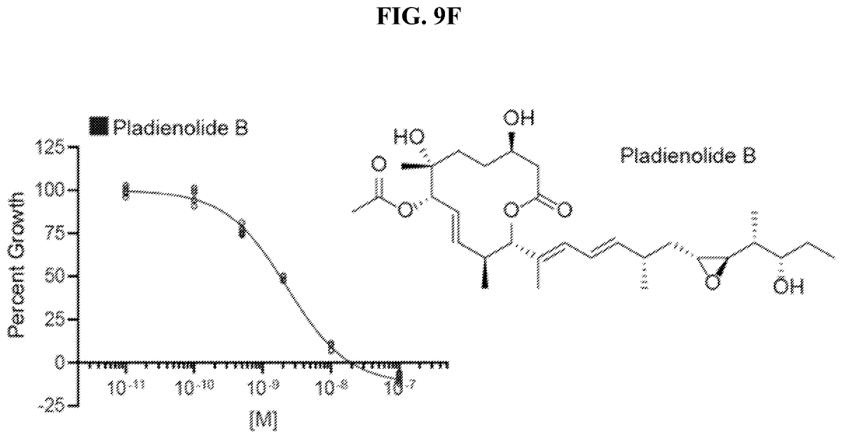

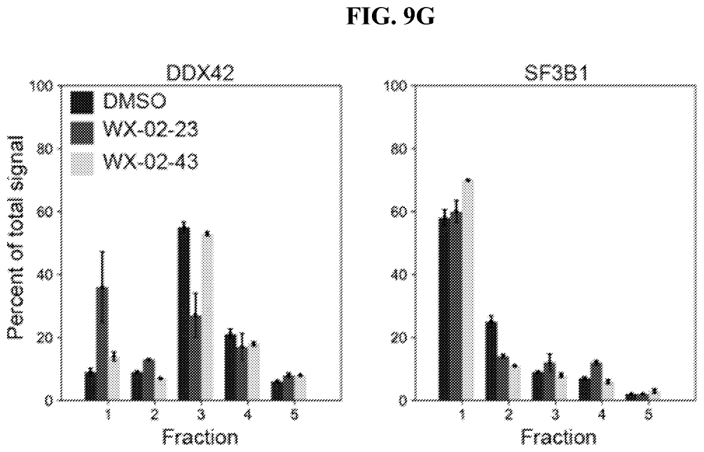

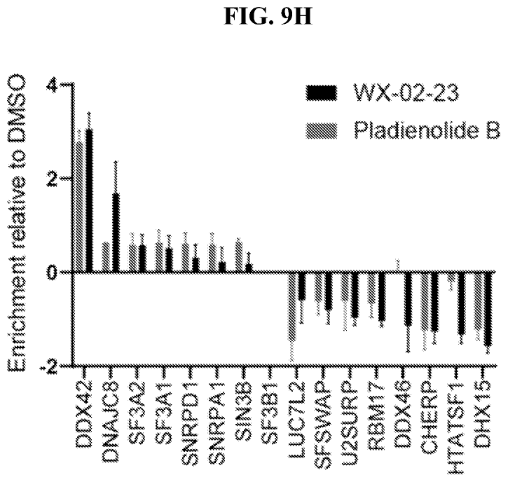

SF3B1 is typically an ˜150 kDa member of the spliceosome. SF3B1 may be essential for stabilizing the branch point adenosine prior to intron removal. Natural product-based modulators of SF3B1 have been described that show broad spectrum anti-proliferative activity. FIGS. 7A-7J and 9A-9H show that electrophilic compounds engaged C1111 of SF3B1 and stereoselectively modulate the spliceosome structure and function. Consistent with SF3B1 being a direct, stereoselective target of tryptoline acrylamides, WX-01-10, but not WX-01-12 labeled an ˜150 kDa protein in 22Rv1 cells and this labeling was blocked by WX-02-23, but not WX-02-43 (FIGS. 7A and 9A). Interestingly, WX-01-10 labeling of the 150 kDa protein was also blocked by pre-treatment with pladienolide B, a natural product modulator of SF3B1 (FIG. 7A). WX-02-23 and pladienolide B also induced expression of p27 (FIG. 7A), a feature of spliceosome modulators that reflects an aberrant splicing event that removes a C-terminal ubiquitination site from p27, which in turn prevents cells from progressing through the G2/M checkpoint.

The co-crystal structure of a pladienolide B-SF3B1 complex has confirmed that this interaction is reversible. Integrating this information with data on the tryptoline acrylamides indicating a covalent mechanism of action and a shared binding pocket with pladienolide B, it was surmised that this pocket might contain a WX-02-23-sensitive cysteine. Review of the SF3B1-pladienolide B structure identified a single Cys1111 in the binding pocket (FIG. 7B). SF3B_C1111 has rarely been quantified in our cysteine reactivity profiling datasets (current and past), suggesting either that this cysteine is poorly reactive with IA probes or that it resides on a nonproteotypic tryptic peptide that is difficult to detect by MS-based proteomics. Consistent with the latter hypothesis, the IA-DTB adduct with a tryptic peptide containing C1111, which is 28 amino acids in length with 14 hydrophobic residues (A, F, I, L, M, V), eluted at the tail end of our standard liquid chromatography gradients (140 min, ˜38% acetonitrile) (FIG. 9B). A targeted proteomic assay was established using parallel reaction monitoring to more consistently quantify the IA-DTB-C1111 tryptic peptide adduct along with several other IA-DTB-labeled cysteines in SF3B1, which revealed that WX-02-23 stereoselectively blocked IA-DTB reaction with C1111 (FIGS. 7C and 9C), but did not affect other cysteines in SF3B1 (FIG. 9D). The tryptic peptide containing C1111 also has another cysteine C1123; however, C1123 was excluded as the possible site of labeling by WX-02-23 by performing trypsin-GluC digests, which showed that the aa 1122-1137 peptide of SF3B1 was unaffected in IA-DTB reactivity in WX-02-23-treated cells (FIG. 9E). Finally, cysteine reactivity profiling data also confirmed that MY-7A and WX-02-23 did not engage PHF5A_C26, which may be targeted by spliceostatin A, another natural product modulator of the spliceosome.

Like MY-7A and WX-02-23, pladienolide B showed strong anti-proliferative activity (FIG. 9F and Table 4), and WX-02-23 and pladienolide B caused strikingly similar changes to the transcriptomes and proteomes of cancer cells as measured by RNA-seq (FIG. 7D) and MS-based proteomics (FIG. 7E), respectively. Data in Table 4 include percent engagement relative to DMSO (higher is more engaged). These changes were not observed with the inactive enantiomer WX-02-43, which instead resembled DMSO-treated cells (FIG. 7F). A deeper analysis of the RNA-seq data revealed that WX-02-23 and pladienolide B also altered mRNA splicing in similar ways, including the induction of both intron retention and exon skipping events (FIGS. 7G and 7H). No such splicing effects were observed with WX-02-43 (FIGS. 7G and 7H).

TABLE 4
Cysteine engagement data from 22Rv1 cells treated
with WX-02-23/43 and digested with GluC + trypsin

| | | 5 uM | 20 uM | 5 uM | 20 uM |
|---|---|---|---|---|---|
| symbol | residue | WX-02-23 | WX-02-23 | WX-02-43 | WX-02-43 |
| PSME1 | 22 | −20.9 | −29.44 | −24.33 | −26.32 |
| PSME1 | 101 | −4.575 | −14.73 | −9.325 | −5.635 |
| PSME1 | 106 | −4.44 | −17.66 | −7.69 | −20.355 |
| SF3B1 | 795 | −4.58 | −5.905 | 5.715 | 3.145 |
| SF3B1 | 796 | −6.42 | −5.075 | 6.755 | 7.34 |
| SF3B1 | 933 | −7.02 | −4.95 | 6.345 | 2.595 |
| SF3B1 | 965 | −15.75 | −8.665 | 1.985 | 13.895 |
| SF3B1 | 1035 | −9.265 | −6.03 | 1.53 | 8.13 |
| SF3B1 | 1123 | 5.965 | 21.005 | 1.425 | 21.84 |
| SF3B1 | 1244 | −11.92 | −12.54 | −1.065 | −1.615 |

To gain further mechanistic insights into the splicing effects caused by small-molecule modulators of SF3B1, the SEC-MS profiles were recalled that show that MY-7A-treated cells exhibited a stereoselective shift in the helicase DDX42 to a higher MW form (FIG. 1H). It was confirmed WX-02-23 also stereoselectively caused this same size shift in DDX42 (FIG. 9G). DDX42 (or SF3b125) can physically associate with the spliceosome, although large-scale immunoprecipitation-MS experiments have not provided evidence that DDX42 is a constitutive member of the spliceosome complex. Fraction 1, to which DDX42 shifted in MY-7A or WX-02-23-treated cells, also contained other spliceosome components, including SF3B1 (FIG. 9G), suggesting that SF3B1 ligands may promote DDX42 binding to the spliceosome. Consistent with this hypothesis, it was found by immunoprecipitation-MS proteomics that DDX42 associated with SF3B1 to a much greater extent in WX-02-23-treated cancer cells compared to DMSO- or WX-02-43-treated cancer cells (FIG. 7I). WX-02-23 also caused a broader remodeling of SF3B1 interactions, including enhanced association with splicing factor DNAJC8 and decreased interactions with several other spliceosome components (FIG. 7I-7J). A subset of proteins displaced from SF3B1 by WX-02-23 have been found to form a distinct functional module of the spliceosome (CHERP, RBM17, U2SURP), suggesting a coordinated reorganization of this complex by SF3B1 ligands. Pladienolide B induced a similar, but not identical reshaping of SF3B1 interactions (FIG. 9H). Taken together, these data indicate that the tryptoline acrylamides MY-7A and WX-02-23 may display stereoselective anti-proliferative activity by modulating spliceosome assembly and function through covalent binding to C1111 of SF3B1.

### DISCUSSION

The pursuit of chemical probes stands to benefit from the introduction of generalized assays that can be applied to diverse types of proteins, preferably, in physiologically relevant settings. Chemical proteomics is an attractive strategy to discover small-molecule binders for proteins in native biological systems. Original chemical proteomic strategies using active site-directed, or activity-based probes produced data where small-molecule binding to a protein could be inferred to cause a functional effect (i.e., a compound discovered to bind the active site of a kinase or hydrolase could be assumed to inhibit this enzyme). As the concepts of activity-based protein profiling have been extended to enable assessment of small-molecule binding far beyond enzyme active sites to include virtually any protein in the human proteome, challenges emerge in relating binding events to functional outcomes. Establishing this connection with assays that assess the functional features of many proteins in parallel can greatly accelerate chemical probe discovery versus a more traditional one-at-a-time investigation of individual small molecule-protein interactions. Introduced is a set of “function-first” proteomic assays aimed at translating a broad swath of small molecule-protein binding events into a subset of interactions that perturb the complexation state, stability, and expression of proteins in cells. Through doing so, chemical probes were discovered that alter the structure and function of key protein complexes involved in proteasomal peptide processing and mRNA splicing in human cells.

The azetidine acrylamides and butynamides described herein that engage C22 of PSME1 appear to represent the first chemical probes targeting the PA28 proteasome regulatory complex. These compounds are useful for assessing the role that the PA28 complex plays in, for instance, MHC class I antigenic peptide processing by the immunoproteasome and cancer resistance mechanisms to proteasome inhibitors. Similarly, the tryptoline acrylamides that engage C1111 of SF3B1 are distinct from other chemical probes described for this protein, which have much more ornate natural product-derived structures. How SF3B1 ligands mechanistically affect spliceosome function may not be fully understood, and the evaluation of analogues of pladienolide B has suggested that these compounds may differentially modulate splicing outcomes. These studies, however, indicate that WX-02-23 and pladienolide B, despite being structurally unrelated and acting though covalent and non-covalent mechanisms, respectively, show strikingly similar global effects on splicing. Thus, the distinct splicing changes caused by individual SF3B1 ligands may be subtle in nature. This may be investigated further, as SF3B1 compounds are in clinical development for cancers, including those with high-frequency mutations in spliceosome components like SF3B1. Toward this end, the discovery that WX-02-23 and pladienolide B remodel the composition of the spliceosome, promoting the association or disassociation of individual subunits, provides insights into leveraging the activity of SF3B1 modulators in a more cancer-restricted manner (e.g., if mutated cancer spliceosomes are found to have unique protein compositions that are differentially affected by SF3B1 ligands).

Reflecting on why the approach described here was successful at identifying selective, cell-active chemical probes that perturb PPIs from a small number of test compounds (two sets of four stereoisomeric acrylamides), a few factors are considered. First, covalent ligands, by leveraging a combination of reactivity and recognition features, may provide an advantage for chemically targeting historically challenging protein classes such as adaptor proteins like PSME1. Second, we believe that the DOS principles of constructing focused compound libraries bearing sp3-rich cores that are stereochemically defined, entropically constrained, and densely functionalized may further improve the probability of engaging PPI sites, at least when compared to more fragment-like and/or sp2-based small-molecule libraries. Also, the function-first screening strategies may themselves accelerate the discovery of chemical probes by prioritizing a subset of covalent liganding events that alter protein complexation state, turnover, and/or expression. In this regard, we further hypothesize that, by focusing on covalent liganding events producing stereoselective functional outcomes, we greatly enriched for chemical probes that act in a site-specific manner. An additional advantage of prioritizing stereoselective functional ligands as chemical probes is that they can be paired with physicochemically matched, inactive enantiomeric control compounds for cell biological studies.

### Experimental Model and Subject Details

### Cell Lines

22Rv1 (ATCC, CRL-2505), MCF7 (ATCC, HTB-22), Ramos (ATCC, CRL-1596), HEK293T (ATCC, CRL-3216) and E.G7-Ova (ATCC, CRL-2113) were grown in in RPMI (22Rv1, Ramos, E.G7-Ova), DMEM (HEK293T), or EMEM (MCF7), supplemented with 10% fetal bovine serum (FBS), 2 mM L-alanyl-L-glutamine (GlutaMAX), penicillin (100 U ml-1), and streptomycin (100 μg ml-1).

Generation of stably transduced clonal 22Rv1 cells expressing TetR: HEK293FT cells (Thermo R70007, supplemented with MEM nonessential amino acids, 25-025-C1 Corning) (5×106) were plated in 10-cm plates and allowed to attach overnight. To 1 mL of serum-free DMEM the following were added: 3 μg of pLenti3.3/TR, 2.25 μg psPAX2.0 plasmid (Addgene, catalog number: 12260) and 0.75 μg CMV VSV-G (Addgene, catalog number: 98286) and 36 μL lipofectamine 2000 transfection reagent (Invitrogen). Reagents were flicked to mix, and after 15 min, the transfection mixture was added dropwise to plates containing cells. Medium was exchanged approximately 16 hours post transfection, virus-containing supernatants were then collected 48 h post transfection and filtered, and then used to infect 22Rv1 cells in 6-well plates in the presence of 6 μg ml-1 Polybrene (Santa Cruz). The media was replaced 24 h later, cells were allowed to recover for an additional 24 h, and then geneticin (400 μg/mL) was added for selection. Clonal pLenti3.3/TR expressing cells were selected by cloning cylinder and subsequently transfected with constructs of interest.

Transfection of epitope tagged and mutant proteins of interest: Clonal pLenti3.3IT R expressing 22Rv1 cells were seeded into 6 cm dishes and 24 h later transfected with proteins of interest cloned into pLenti6.3. One μg of pLenti6.3 plasmid and 3 μL of PEI (1 μg/μL) were added to 200 μL serum-free RPMI and incubated for 15 minutes. Medium of cells was replaced to contain tetracycline (0.1-1 μg/mL final concentration) and then transfection mixture was added dropwise. Cells were assayed 24-48 h later.

### Experimental Methods

### Proteomic Platforms: SEC-MS

Sample preparation: Cells (adherent: 15 cm dishes, suspension: 2 million cells/mL) were treated with electrophilic probes in situ for indicated times and concentrations (elaborated fragments: 20 μM, 3 h), washed with ice-cold PBS, and flash frozen in liquid nitrogen. Cell pellets were lysed by sonication (8 pulses, 40% power) in 600 μL ice-cold PBS supplemented with cOmplete protease inhibitors (1 tablet/10 mL of PBS), ultracentrifuged for 20 min at 100,000 g, then normalized to a standardized concentration (typically 1.5 to 2.5 mg/mL). Clarified lysate was injected into a Superdex 200 Increase 10/300 GL column attached to an AKTA pure FPLC system. Proteins were fractionated using an isocratic gradient (PBS) running at 0.5 mL/min into 5 fractions 2 mL wide, beginning at 8 mL and ending at 18 mL. Eluate was collected into 15 mL tubes containing 12 mL of acetone at 4C. After completion of one set of 5 fractions, the above protocol was repeated a second time for the remaining 5 fractions. Proteins were precipitated overnight at −20 C, then spun at 4500 g for 20 minutes, yielding a white protein pellet. Acetone/PBS mixture was decanted off the pellets, which were dried at room temperature and then resuspended in 125 μL of 8 M urea, 10 mM DTT in 100 mM EPPS for 15 minutes at 65 degrees, followed by probe sonication to complete resuspension. 6.25 μL of 500 mM iodoacetamide was added to samples for alkylation (30 minutes, 37 C), followed by dilution with 370 μL of 100 mM EPPS, and addition of 2 μg trypsin per fraction, and digestion overnight at 37 C. 70 μL of tryptic digest was aliquoted into a clean microcentrifuge tube, followed by 30 μL of dry acetonitrile and 4.5 μL (5 mg/256 μL) of TMT tag in dry acetonitrile. Samples were labeled at room temperature for 75 minutes, then quenched with 6 μL of 5% hydroxylamine for 15 minutes and acidified with 5 μL of formic acid. The set of 10 samples were then combined into a single tube and evaporated by speed vac. Samples were then desalted and fractionated before analysis by mass spectrometry.

Data processing: Fractional distributions for each peptide-spectra match with a total TMT reporter ion intensity were calculated by dividing the reporter ion intensity for each TMT channel by the summed intensity across the 5 TMT channels corresponding to a single treatment condition (Equation 1). Protein-level SEC elution profiles were then generated by averaging together all unique peptides with a summed reporter ion intensity >5,000. Two unique peptide sequences were required per-protein. Bar graph elution profiles are represented as the mean±standard error of the mean across all replicates. Euclidean distances (SE shift scores, Equation 2) were calculated using the average elution profile for each protein across replicates, combined by treatment condition and cell line. Figures reporting mean elution times used Equation 3 for calculation.

Filtering of SEC-MS data: Replicates were combined based on cell line and treatment condition (probe, concentration, duration) by averaging SEC elution profiles for each protein across all experiments. A coefficient of variation (CV) filter was applied by taking the average the per-fraction CV across all 5 fractions. Proteins with a CV≥0.7 were removed from analysis.

$$\begin{matrix}{X}_{i} = \frac{Reporter Ion {Intensity}_{i}}{{\sum }_{j = 1}^{5} {Intensity}_{j}} & Equation 1 \\ SE Shift ( X , Y ) = \sqrt{{\sum }_{i = 1}^{5} {( {X}_{i} - {Y}_{i} )}^{2}} & Equation 2 \\ Mean elution time ( X ) = \frac{{\sum }_{i = 1}^{n} i * {X}_{i}}{100} & Equation 3\end{matrix}$$

In Equations 1-3, Xᵢ and Yi represent protein-level fractional distributions for individual treatment conditions.

### SEC-MS Analysis by Western Blot

For experiments using transiently overexpressed constructs (FLAG-PSME1 WT/C22A, HA-ACAT1 WT/C126A), Stable clonal 22Rv1 pLenti3.3/TR were transfected using polyethylenimine and treated with tetracycline (0.1-1.0 mg/mL) for 48 h prior to treatment with electrophilic probes.

Cells were lysed and fractioned by SEC. After acetone precipitation and resuspension, 4× gel loading buffer was added to eluates, and proteins were resolved using SDS-PAGE (4-20% acrylamide, Tris-glycine gel), and transferred to a nitrocellulose membrane. The membrane was blocked with 5% milk in Tris-buffered saline (20 mM Tris-HCl 7.6, 150 mM NaCl) with 0.1% tween (TBST) and incubated with primary antibody overnight. After TBST wash (3 times), the membrane was incubated with secondary antibody, washed with TBST again, developed with ECL western blotting detection reagents, and recorded on a Bio-Rad ChemiDoc MP. Relative band intensities were analyzed using ImageJ.

### Proteomic Platform: Cysteine Ligandability Profiling

Sample preparation: Cells (adherent: 15 cm dishes, suspension: 2 million cells/mL) were treated with electrophilic probes in situ for indicated times and concentrations, washed with ice-cold PBS, and flash frozen in liquid nitrogen. Cell pellets were lysed by sonication (8 pulses, 40% power) in 500 μL ice-cold PBS supplemented with cOmplete protease inhibitors (1 tablet/10 mL of PBS). Whole cell lysates protein content was measured using a standard DC protein assay (Bio-Rad) and 500 μL (2 mg/mL protein content) were treated with 5 μL of 10 mM 1A-DTB (in DMSO) for 1 h at ambient temperature with occasional vortexing. Samples were methanol-chloroform precipitated with the addition of 500 μL ice-cold MeOH and 200 μL CHCl3, vortexed, and centrifuged (10 min, 10,000 g). Without disrupting the protein disk, both top and bottom layers were aspirated and the protein disk was washed with 1 mL ice-cold MeOH and centrifuged (10 min, 10,000 g). The pellets were allowed to air dry, and then re-suspended in 90 μL buffer (9 M urea, 10 mM DTT, 50 mM TEAB pH 8.5). Samples were reduced by heating at 65 C for 20 minutes and water bath sonicated as needed to resuspend the protein pellets, followed by alkylation via addition of 10 μL (500 mM) iodoacetamide and incubated at 37 C for 30 min with shaking. Samples were briefly centrifuged, and probe sonicated once more to ensure complete resuspension, and then diluted with 300 μL buffer (50 mM TEAB pH 8.5) to reach final concentration of 2 M urea. Trypsin (4 μL of 0.25 μg/μL in trypsin resuspension buffer with 25 mM CaCl₂)) was added to each sample and digested at 37 C for 2 h to overnight. Digested samples were then diluted with 300 μL wash buffer (50 mM TEAB pH 8.5, 150 mM NaCl, 0.2% NP-40) containing streptavidin-agarose beads (50 μL of 50% slurry in wash buffer) and were rotated at room temperature for 2 hours. Enriched samples were pelleted by centrifugation (2000 g, 2 min) and transferred to BioSpin columns and washed (3×1 mL wash buffer, 3×1 mL PBS, 3×1 mL water). Enriched peptides were eluted by 300 μL 50% acetonitrile with 0.1% formic acid and eluate was evaporated to dryness via speedvac. IA-DTB labeled and enriched peptides were resuspended in 100 μL EPPS buffer (200 mM, pH 8.0) with 30% acetonitrile, vortexed, and water bath sonicated. Samples were TMT labeled with 3 μL of corresponding TMT tag (5 mg tag resuspended in 256 μL acetonitrile), vortexed, and incubated at room temperature for 1 hour. TMT labeling was quenched with the addition of hydroxylamine (5 μL 5% solution in H2O) and incubated for 15 minutes at room temperature. Samples were then acidified with 5 μL formic acid, combined and dried using a SpeedVac. Samples were desalted via Sep-Pak and then high pH fractionated.

Sample preparation for denatured condition: Samples were treated as described above, with the exception that for 1A-DTB labeling, samples (1 mg per condition) were denatured in 8 M urea and heated at 65 C for 15 minutes and allowed to cool prior to addition of 5 μL of 10 mM 1A-DTB (in DMSO) for 1 h at ambient temperature with occasional vortexing and then processed.

Data processing: Cysteine engagement ratios (probe vs DMSO) were calculated for each peptide-spectra match by dividing each TMT reporter ion intensity by the average intensity for the channels corresponding to DMSO treatment. Peptide-spectra matches were then grouped based on protein ID and residue number (e.g., PSME1 C22), excluding peptides with summed reporter ion intensities for the DMSO channels <10,000, coefficient of variation for DMSO channels >0.5, and non-tryptic peptide sequences. TMT reporter ion intensities were normalized to the median summed signal intensity across channels.

Filtering of cysteine data: Replicates were combined based on cell line and treatment condition (probe, concentration, duration) by averaging cysteine engagement ratios across all experiments.

$$R value = \frac{100}{100 - \% engagement relative to DMSO} , clipped between 0 and 10 \qquad Equation 4$$

### Proteomic Platform: Whole Proteome Profiling

Sample preparation: Cells (adherent: 15 cm dishes, suspension: 2 million cells/mL) were treated with electrophilic probes in situ for indicated times and concentrations (elaborated compounds: 1 hr or 3 hr as noted), washed with ice-cold PBS, and flash frozen in liquid nitrogen. Cell pellets were lysed by sonication (8 pulses, 40% power) in 200 μL ice-cold PBS supplemented with cOmplete protease inhibitors (1 tablet/10 mL of PBS) and 1 mM PMSF (100 mM stock in ethanol). Whole cell lysates protein content was measured using a standard DC protein assay (Bio-Rad) and a volume corresponding to 200 μg was transferred to a new low-bind Eppendorf tube (containing 48 mg urea) and brought to 100 μL total volume. Samples were reduced with DTT (5 μL 200 mM stock in H2O, 10 mM final concentration) and incubated at 65C for 15 minutes, then alkylated with iodoacetamide (5 μL 400 mM stock in H2O, 20 mM final concentration) incubated at 37C for 30 minutes in the dark. Samples were precipitated with the addition of ice-cold MeOH (600 μL), CHCl3 (180 μL), and H2O (500 μL), and vortexed and centrifuged (16,000 g, 10 minutes, 4 C). The top layer above the protein disc was aspirated and an additional 1 mL of ice-cold MeOH was added. The samples were again vortexed and centrifuged (16,000 g, 10 minutes, 4 C), the supernatant aspirated and the protein pellets were allowed to air dry and be stored at −80 C or proceeded to resuspension and digestion. Samples were resuspended in 160 μL EPPS buffer (200 mM pH 8.0) using a probe sonicator (10-15 pulses). Proteomes were first digested with LysC (4 μL per sample, 0.5 μg/μL in HPLC grade water) for 2 h at 37 C. Then trypsin (8 μL per sample, 0.5 μg/μL in trypsin resuspension buffer with 20 mM CaCl₂)) was added and samples were incubated at 37 C overnight. Peptide concentrations were estimated using microBCA assay (Thermo Scientific), and a volume corresponding to 25 μg was transferred to a new low-bind Eppendorf tube and brought to 35 μL with EPPS buffer. Samples were diluted with 9 μL acetonitrile and then TMT labeled with 5 μL of corresponding TMT tag (5 mg tag resuspended in 256 μL acetonitrile), vortexed, and incubated at room temperature for 1 hour. TMT labeling was quenched with the addition of hydroxylamine (5 μL 5% solution in H2O) and incubated for 15 minutes at room temperature. Samples were then acidified with 2.5 μL formic acid and an equal volume (20 μL corresponding to ˜8.85 μg per channel) was combined and dried using a SpeedVac. Samples were desalted via Sep-Pak and then high pH fractionated.

Data processing: Protein abundance ratios (probe vs DMSO) were calculated for each peptide-spectra match by dividing each TMT reporter ion intensity by the average intensity for the channels corresponding to DMSO treatment. Peptide-spectra matches were then grouped based on protein ID, excluding peptides with summed reporter ion intensities for the DMSO channels <10,000, coefficient of variation for DMSO channels >0.5, non-unique or non-tryptic peptide sequences. TMT reporter ion intensities were normalized to the median summed signal intensity across channels.

Filtering of whole proteome data: Whole proteome data was filtered at peptide-level by removing any peptide-spectra matches with a standard deviation of abundance ratio >100%. At a replicate-level, proteins with at least 2 distinct peptide-spectra matches were retained for analysis. Replicates were then combined based on cell line and treatment condition (probe, concentration, duration) by averaging protein abundance ratios across all experiments.

### Offline Fractionation

High pH offline fractionation was performed. Samples fractionated via Peptide Desalting Spin Columns (Thermo 89852) were resuspended in buffer A (5% acetonitrile, 0.1% formic acid) and bound to the spin columns. Bound peptides were then washed in water, 10 mM NH4HCO3 containing 5% acetonitrile, and eluted in fractions of increasing acetonitrile, concatenated, and then dried using a SpeedVac vacuum concentrator. Resulting fractions were resuspended in buffer A (5% acetonitrile, 0.1% formic acid) and analyzed by mass spectrometry.

Samples fractionated by HPLC were resuspended in buffer A (500 μL) and fractionated into a 96 deep-well plate using HPLC (Agilent). The peptides were eluted onto a capillary column (ZORBAX 300Extend-C18, 3.5 μm) and separated at a flow rate of 0.5 mL/min using the following gradient: 100% buffer A from 0-2 min, 0%-13% buffer B from 2-3 min, 13%-42% buffer B from 3-60 min, 42%-100% buffer B from 60-61 min, 100% buffer B from 61-65 min, 100%-0% buffer B from 65-66 min, 100% buffer A from 66-75 min, 0%-13% buffer B from 75-78 min, 13%-80% buffer B from 78-80 min, 80% buffer B from 80-85 min, 100% buffer A from 86-91 min, 0%-13% buffer B from 91-94 min, 13%-80% buffer B from 94-96 min, 80% buffer B from 96-101 min, and 80%-0% buffer B from 101-102 min (buffer A: 10 mM aqueous NH4HCO3; buffer B: acetonitrile). Each well in the 96-well plate contained 20 μL of 20% formic acid to acidify the eluting peptides. The eluent was evaporated to dryness in the plate using SpeedVac vacuum concentrator. The peptides were resuspended in 80% acetonitrile, 0.1% formic acid buffer (125 μL/column) and every 12th fraction was combined. Samples were dried using SpeedVac vacuum concentrator, the resulting 12 combined fractions were re-suspended in buffer A (5% acetonitrile, 0.1% formic acid) and analyzed by mass spectrometry.

### Parallel Reaction Monitoring

Dry peptide samples were reconstituted in a water/acetonitrile (85:15) mixture containing 0.1% formic acid (100 μl) and 15 μl was injected on to an EASY-Spray C18 loading column (5 μm particle size, 100 μm×2 cm; Fisher Scientific, DX164564) and resolved on a custom analytical column (2 μM particle size, 75 μm×15 cm) using a Dionex Ultimate 3000 nano-LC (Thermo Fisher Scientific). Peptides were separated over a 60-min gradient of 15 to 33% acetonitrile (0.1% formic acid) and analyzed on a Q-Exactive instrument (Thermo Fisher Scientific) using a parallel reaction monitoring method targeting the SF3B1 peptide containing C1111 (amino acids 1110-1149, missed tryptic, +3 charge state). Selected precursor ions were isolated and fragmented by high-energy collision dissociation and fragments were detected in the Orbitrap at 17,500 resolution.

Raw data files were uploaded analyzed in Skyline (v.21.1.0.278) to determine the abundance of each peptide in vehicle-treated samples relative to inhibitor-treated samples. Peptide quantification was performed by calculating the sum of the peak areas corresponding to six fragment ions from each peptide. The peptides and fragment ions were preselected from in-house reference spectral libraries acquired in data-dependent acquisition mode to identify authentic spectra for each peptide.

### TMT Liquid Chromatography-Mass-Spectrometry (LC-MS) Analysis

Samples were analyzed by liquid chromatography tandem mass-spectrometry using an Orbitrap Fusion mass spectrometer (Thermo Scientific) coupled to an UltiMate 3000 Series Rapid Separation LC system and autosampler (Thermo Scientific Dionex). The peptides were eluted onto a capillary column (75 μm inner diameter fused silica, packed with C18 (Waters, Acquity BEH C18, 1.7 μm, 25 cm)) or an EASY-Spray HPLC column (Thermo ES902, ES903) using an Acclaim PepMap 100 (Thermo 164535) loading column, and separated at a flow rate of 0.25 μL/min. Data was acquired using an MS3-based TMT method on Orbitrap Fusion or Orbitrap Eclipse Tribrid Mass Spectrometers. The scan sequence began with an MS1 master scan (Orbitrap analysis, resolution 120,000, 400-1700 m/z, RF lens 60%, automatic gain control [AGC] target 2E5, maximum injection time 50 ms, centroid mode) with dynamic exclusion enabled (repeat count 1, duration 15 s). The top ten precursors were then selected for MS2/MS3 analysis. MS2 analysis consisted of: quadrupole isolation (isolation window 0.7) of precursor ion followed by collision-induced dissociation (CID) in the ion trap (AGC 1.8E4, normalized collision energy 35%, maximum injection time 120 ms). Following the acquisition of each MS2 spectrum, synchronous precursor selection (SPS) enabled the selection of up to 10 MS2 fragment ions for MS3 analysis. MS3 precursors were fragmented by HCD and analyzed using the Orbitrap (collision energy 55%, AGC 1.5E5, maximum injection time 120 ms, resolution was 50,000). For MS3 analysis, we used charge state-dependent isolation windows. For charge state z=2, the MS isolation window was set at 1.2; for z=3-6, the MS isolation window was set at 0.7. The MS2 and MS3 files were extracted from the raw files using RAW Converter (version 1.1.0.22; available at fields.scripps.edu/rawconv/), uploaded to Integrated Proteomics Pipeline (IP2), and searched using the ProLuCID algorithm (publicly available at fields.scripps.edu/downloads.php) using a reverse concatenated, non-redundant variant of the Human UniProt database (release 2016-07). Cysteine residues were searched with a static modification for carboxyamidomethylation (+57.02146 Da). For cysteine profiling experiments, a dynamic modification for 1A-DTB labeling (+398.25292 Da) was included with a maximum number of 2 differential modifications. N-termini and lysine residues were also searched with a static modification corresponding to the TMT tag (+229.1629 Da). Peptides were required to be at least 6 amino acids long, and to be fully tryptic, except for the GluC digested cysteine profiling samples which included K, R, E, and D cleavage sites. ProLuCID data was filtered through DTASelect (version 2.0) to achieve a peptide false-positive rate below 1%. The MS3-based peptide quantification was performed with reporter ion mass tolerance set to 20 ppm with Integrated Proteomics Pipeline (IP2).

### Protein Abundance Analysis by Western Blot

For experiments using transiently overexpressed constructs (HA-ACAT1 WT/C₁₂₆A, HA-AR WT/8-mutant, and individual mutants), stable clonal 22Rv1 pLenti3.3/TR were transfected using polyethylenimine and treated with tetracycline (0.1-1.0 mg/mL) for 24 hr prior to treatment with electrophilic probes.

Cells were lysed and whole cell lysates protein content was measured using a standard DC protein assay (Bio-Rad) and a volume corresponding to 20 μg was transferred to a new low-bind Eppendorf tube, 4× gel loading buffer was added to samples, and proteins were resolved using SDS-PAGE (4-12% acrylamide, Tris-glycine gel), and transferred to a nitrocellulose membrane (350 mA for 90 min). The membrane was blocked with 5% milk in Tris-buffered saline (20 mM Tris-HCl 7.6, 150 mM NaCl) with 0.1% tween (TBST) and incubated with primary antibody overnight. After TBST wash (3 times), the membrane was incubated with secondary antibody, washed with TBST again, developed with ECL western blotting detection reagents (Supersignal West Femto Maximum Sensitivity Substrate), and recorded on a Bio-Rad ChemiDoc MP. Relative band intensities were analyzed using ImageLab.

### Bioinformatic Analysis of Protein Complexes

The CORUM Core Complex database (version 3.0) was used as a “gold standard” set of protein complexes to benchmark SEC-MS against another method by Kirkwood et al. Cytoscape version 3.8.2 was used to generate PPI networks, and edge weights were calculated by taking the absolute difference of SEC mean elution times between treatment conditions, or with fold-change in enrichment by anti-SF3B1.

### Gel-Based ABPP for PSME1 Cysteine Engagement

C-terminally FLAG-tagged PSME1 WT or C22A were transiently expressed in HEK293T cells, harvested 48 hours later, then cells were divided into aliquots, flash frozen and stored at −80 C. On the day of the experiment, an aliquot of cells were thawed, lysed by sonication in ice-cold PBS supplemented with cOmplete protease inhibitors (1 tablet/10 mL of PBS). normalized to 2.0 mg/mL, and divided into 25 μL aliquots. 1 μL of DMSO or compound (25× stock) was incubated with lysates for 1.5 hours at RT, followed by incubation with 1 μL of 62.5 μM MY-11B click probe (final concentration 2.4 μM) for an additional 30 minutes. Reagents for the CuAAC click reaction were pre-mixed prior to addition to the samples. After 1 hour of click labeling, 4×SDS running buffer was added to samples, which were then analyzed by SDS-PAGE. ImageJ was used to quantify rhodamine band intensities, and GraphPad PRISM Version 9.0.0 was used to generate IC50 curves (four-parameter variable slope least squares regression).

FACS Analysis with Citric Acid Wash

E.G7-Ova cells (2 million/mL) were treated with compounds for 4 hours, washed with PBS, and then washed with 1 mL of 131 mM citric acid for 2 minutes, quenched with 1 mL 66 mM NaH2PO4, and then washed with PBS 3 times. Cells were then resuspended in warm RPMI and allowed to recover for the indicated period, after which cells were washed with PBS and transferred to 96 well plates for staining. Each well was washed with 200 μL PBS, and then stained for 20 minutes with 50 μL of staining solution: 1:1000 fixable near-IR LIVE/DEAD stain (Invitrogen) and 1:200 dilution of anti-SIINFEKL (BioLegend) or anti-Kb (BioLegend) antibody in FACS buffer (2% FBS in PBS supplemented with PenStrep and sterile filtered). Additional wells were used as unstained or singly-stained controls. After staining, cells were washed with PBS, then fixed in 200 μL 4% paraformaldehyde in PBS for 15 minutes. Cells were resuspended in 75 μL FACS buffer and analyzed on a Novocyte flow cytometer. The corresponding data in FIG. 2J are presented as the average percentage of median fluorescence intensity relative to DMSO treated control±SD from n=4 replicates. The corresponding data in FIG. 2K are presented as the average percentage of MFI relative to DMSO treated control±SD from n=3 replicates. Statistical analysis was performed using GraphPad PRISM Version 9.0.0.

### Proliferation Assay

Cells were seeded at 5000 cells per well (50 μL medium) in 96-well flat bottom white wall plates. After 24 h, 50 μL of medium containing DMSO or compound dilutions (2× final concentration from 1000× stocks) were added to the wells. At the time of compound addition, a reference plate to determine the cell population density at time 0 (TO) was assayed using CellTiter Glo reagent (50 μL added to each well). After 72 h in culture, the remaining plates were assayed using CellTiter Glo. After 30 min of shaking at room temperature, luminescence was analyzed using a CLARIOstar (BMG Labtech) plate reader. Raw values were normalized using GraphPad PRISM software version 9.0.0. Lines of best fit were generated using a four-parameter variable slope least squares regression.

### MS-Based ABPP for SF3B1 Cysteine Engagement

22Rv1 cells were treated with indicated compounds for 2 h, chased with click probe for 1 h in situ, harvested and lysed in PBS with protease inhibitors (cOmplete), and then normalized to 2.0 mg/mL. Reagents for the CuAAC click reaction were pre-mixed prior to addition to the samples (55 μL of click reaction mix for 500 μL of lysate). After 1 h of click labeling, samples were precipitated with the addition of ice-cold MeOH (600 μL), CHCl3 (180 μL), and H₂O (500 μL), and vortexed and centrifuged (16,000 g, 10 minutes, 4 C). The top layer above the protein disc was aspirated and an additional 1 mL of ice-cold MeOH was added. The samples were again vortexed and centrifuged (16,000 g, 10 minutes, 4 C), the supernatant aspirated and the protein pellets were allowed to air dry and be stored at −80 C or proceeded to resuspension and enrichment. Pellets were resuspended in 500 μL 8 M urea (in DPBS) with 10 μL 10% SDS and sonicated to ensure no large precipitate remained. Samples were reduced with 25 μL of 200 mM DTT and incubated at 65 C for 15 minutes, then reduced with addition of 25 μL of 400 mM iodoacetamide and incubated in the dark at 37 C for 30 minutes. For enrichment, 130 μL 10% SDS was added and then samples were diluted to 5.5 mL DPBS. Samples were enrichment with streptavidin beads (Thermo cat #20353; 100 μL/sample) and incubated at room temperature for 1.5 h while rotating. After incubation, samples were pelleted by centrifugation (2,000 g, 2 min) and beads were washed with 0.2% SDS in DPBS (2×10 mL), DPBS (1×5 mL), then transferred to low-bind Eppendorf (cat #0030108442), washed with water (2×1 mL), and 200 mM EPPS (1 mL). Pelleted beads were resuspended in 200 μL 2 M urea (in 200 mM EPPS) and digested with 2 μg Trypsin with 1 mM CaCl₂) (final concentration) overnight at 37 C. Digested peptides were transferred to new low-bind Eppendorf and acetonitrile added (to 30% final) and TMT labeled with 6 μL of corresponding TMT tag (20 μg/μL), vortexed, and incubated at room temperature for 1 h. TMT labeling was quenched with the addition of hydroxylamine (6 μL 5% solution in H₂O) and incubated for 15 minutes at room temperature. Samples were then acidified with 15 μL formic acid, combined and dried using a SpeedVac. Samples were desalted and high pH fractionated using Peptide Desalting Spin Columns (Thermo 89852) to a final of 3 fractions and analyzed by mass spectrometry.

### Gel-Based ABPP for SF3B1 Cysteine Engagement

22Rv1 cells were treated with indicated compounds for 24 h, chased with click probe for 1 h in situ, harvested and lysed in PBS with protease inhibitors (cOmplete), and then normalized to 2.0 mg/mL. Reagents for the CuAAC click reaction were pre-mixed prior to addition to the samples (3 μL of click reaction mix for 25 μL of lysate). After 1 hour of click labeling, 4×SDS running buffer was added to samples, which were then analyzed by SDS-PAGE.

RNA Sequencing and Analysis of differential Gene Expression

RNA sequencing libraries were prepared using the NEBNext Ultra II RNA Library Prep Kit for Illumina following manufacturer's instructions (NEB, Ipswich, MA, USA). mRNAs were first enriched with Oligo(dT) beads. Enriched mRNAs were fragmented for 15 minutes at 94° C. First strand and second strand cDNAs were subsequently synthesized. cDNA fragments were end repaired and adenylated at 3′ends, and universal adapters were ligated to cDNA fragments, followed by index addition and library enrichment by limited-cycle PCR. The sequencing libraries were validated on the Agilent TapeStation (Agilent Technologies, Palo Alto, CA, USA), and quantified by using Qubit 2.0 Fluorometer (Invitrogen, Carlsbad, CA) as well as by quantitative PCR (KAPA Biosystems, Wilmington, MA, USA). FASTQ files were first trimmed using Trim_galore (v0.6.4) to remove sequencing adapters and low quality (Q<15) reads. Trimmed sequencing reads were aligned to the human Hg19 reference genome (GENCODE, GRCh37.p13) using STAR (v2.7.5). SAM files were subsequently converted to BAM files, sorted, and indexed using samtools (v1.9). BAM files were used to generate bigwig files using bamCoverage (part of the Deeptools package; v3.3.1). Read counting across genomic features was performed using featureCounts (part of the subread package; v1.5.0) using the following parameters: -p -T 20 -O -F GTF -t exon. Differential gene expression analysis was performed using the edgeR (v3.32.1), DESeq2 (v1.30.1), and limma voom (v3.46.0) R packages. Pre-ranked gene set enrichment analyses were performed using GSEA software (Broad Institute) against the Molecular Signatures Database (MSigDB) collection. Data visualization and figure generation was performed in Rstudio (v1.3.1073) using the following packages: ggplot2 (v3.3.5), ggpubr (v0.4.0), complexHeatmap (v2.6.2), and VennDiagram (v1.6.0).

For quantification of alternative RNA splicing, BAM files generated by STAR/Samtools were analyzed using rMATS (v4.1.1) using the GENCODE (v19) GTF annotation for Hg19 (GRCh37.p13) and the following parameters: -t paired --libType fr-unstranded—readLength 150—novelSS. To utilize reads shorted than 150b.p. resulting from adapter and/or QC trimming by trim_galore, rMATS was programmed to accept soft-clipped reads of variable length. Enumeration of isoform counts was performed using only reads that span the splice junction directly. To identify high confidence AS events, events were considered significant if (i) the inclusion level difference was greater than 20% compared to DMSO, (ii) the False Discovery Rate (FDR) was smaller than 0.05, and (iii) there were a minimum of 20 reads mapping to the splice junction. Data analysis and visualization was performed using custom scripts in Rstudio (v1.3.1073) using the following packages: ggplot2 (v3.3.5), ggrepel (v), maser (v), and VennDiagram (v1.6.0).

### SF3B1 Co-Immunoprecipitation Studies

HEK293T cells were treated in situ with DMSO, 5 μM of WX-02-23/WX-02-43, or 10 nM pladienolide B for 3 hours. Cells were harvested, washed with ice-cold PBS, and lysed in ColP lysis buffer (0.5% NP-40, 100 mM EPPS, 150 mM NaCl, cOmplete protease inhibitor). Lysates centrifuged at 16,000 g for 3 minutes, normalized to 2.0 mg/mL and 500 μL of each lysate was aliquoted to a clean eppendorf tube.

Lysates were incubated with 5 μL of anti-IgG or anti-SF3B1 (CST) for 2 hours at 4 C, followed by 1 hour with 25 μL of protein-A agarose beads. Bead were washed twice with ColP lysis buffer, and then twice with 100 mM EPPS, followed by elution with 8 M urea in EPPS at 65 C for 10 min. Eluates were reduced with DTT at 65 C for 15 min (2.5 μL of 200 mM=12.5 mM final), alkylated with iodoacetamide at 37 C for 30 min (2.5 μL of 400 mM=25 mM final), and diluted to 2 M urea by addition of 115 μL of EPPS. Samples were then trypsinized at 37 C overnight (2 ug of trypsin per sample), then TMT labeled and desalted.

### Quantification and Statistical Analysis

Unless otherwise stated, quantitative data are expressed in bar and line graphs as mean±SD (error bar) shown. Protein elution profiles are expressed as mean SEM (error bar). Unless otherwise stated, differences between two groups were examined using an unpaired two-tailed Student's t-test with equal or unequal variance as noted. Significant P values are indicated (*P<0.05, **P<0.01, ***P<0.001, and ****P<0.0001).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCES

| SEQ | |
|---|---|
| ID NO: | Description and sequence |
| 1 | UniProtKB - 075533 (SF3B1_HUMAN) Isoform 1 |
| | (075533-1): |
| | MAKIAKTHEDIEAQIREIQGKKAALDEAQGVGLDSTGYYDQEIYGGSDSRF |
| | AGYVTSIAATELEDDDDDYSSSTSLLGQKKPGYHAPVALLNDIPQSTEQYD |
| | PFAEHRPPKIADREDEYKKHRRTMIISPERLDPFADGGKTPDPKMNARTYM |
| | DVMREQHLTKEEREIRQQLAEKAKAGELKVVNGAAASQPPSKRKRRWDQTA |
| | DQTPGATPKKLSSWDQAETPGHTPSLRWDETPGRAKGSETPGATPGSKIWD |
| | PTPSHTPAGAATPGRGDTPGHATPGHGGATSSARKNRWDETPKTERDTPGH |
| | GSGWAETPRTDRGGDSIGETPTPGASKRKSRWDETPASQMGGSTPVLTPGK |
| | TPIGTPAMNMATPTPGHIMSMTPEQLQAWRWEREIDERNRPLSDEELDAMF |
| | PEGYKVLPPPAGYVPIRTPARKLTATPTPLGGMTGFHMQTEDRTMKSVNDQ |
| | PSGNLPFLKPDDIQYFDKLLVDVDESTLSPEEQKERKIMKLLLKIKNGTPP |
| | MRKAALRQITDKAREFGAGPLFNQILPLLMSPTLEDQERHLLVKVIDRILY |
| | KLDDLVRPYVHKILVVIEPLLIDEDYYARVEGREIISNLAKAAGLATMIST |
| | MRPDIDNMDEYVRNTTARAFAVVASALGIPSLLPFLKAVCKSKKSWQARHT |
| | GIKIVQQIAILMGCAILPHLRSLVEIIEHGLVDEQQKVRTISALAIAALAE |
| | AATPYGIESFDSVLKPLWKGIRQHRGKGLAAFLKAIGYLIPLMDAEYANYY |
| | TREVMLILIREFQSPDEEMKKIVLKVVKQCCGTDGVEANYIKTEILPPFFK |
| | HFWQHRMALDRRNYRQLVDTTVELANKVGAAEIISRIVDDLKDEAEQYRKM |
| | VMETIEKIMGNLGAADIDHKLEEQLIDGILYAFQEQTTEDSVMLNGFGTVV |
| | NALGKRVKPYLPQICGTVLWRLNNKSAKVRQQAADLISRTAVVMKTCQEEK |
| | LMGHLGVVLYEYLGEEYPEVLGSILGALKAIVNVIGMHKMTPPIKDLLPRL |
| | TPILKNRHEKVQENCIDLVGRIADRGAEYVSAREWMRICFELLELLKAHKK |
| | AIRRATVNTFGYIAKAIGPHDVLATLLNNLKVQERQNRVCTTVAIAIVAET |
| | CSPFTVLPALMNEYRVPELNVQNGVLKSLSFLFEYIGEMGKDYIYAVTPLL |
| | EDALMDRDLVHRQTASAVVQHMSLGVYGFGCEDSLNHLLNYVWPNVFETSP |
| | HVIQAVMGALEGLRVAIGPCRMLQYCLQGLFHPARKVRDVYWKIYNSIYIG |
| | SQDALIAHYPRIYNDDKNTYIRYELDYIL |
| 2 | UniProtKB - Q06323 (PSME1_HUMAN) Isoform 1 |
| | (identifier: Q06323-1): |
| | MAMLRVQPEAQAKVDVFREDLCTKTENLLGSYFPKKISELDAFLKEPALNE |
| | ANLSNLKAPLDIPVPDPVKEKEKEERKKQQEKEDKDEKKKGEDEDKGPPCG |
| | PVNCNEKIVVLLQRLKPEIKDVIEQLNLVTTWLQLQIPRIEDGNNFGVAVQ |
| | EKVFELMTSLHTKLEGFHTQISKYFSERGDAVTKAAKQPHVGDYRQLVHEL |
| | DEAEYRDIRLMVMEIRNAYAVLYDIILKNFEKLKKPRGETKGMIY |

### Synthesis of Probes

### Synthesis of MY-1A

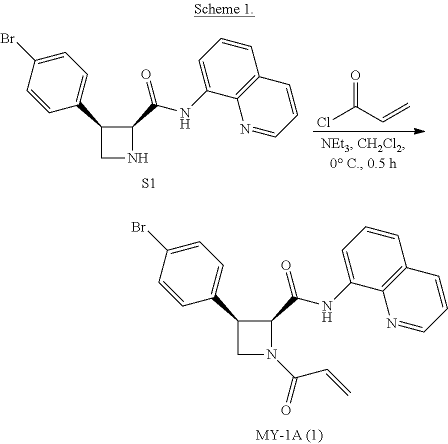

### (2S,3R)-1-acryloyl-3-(4-bromophenyl)-N-(quinolin-8-yl)azetidine-2-carboxamide (MY-1A)(1)

To a precooled (0° C.) solution of S1 (50.0 mg, 131 μmol) (Maetani et al., 2017) in dichloromethane (1 mL) were added triethylamine (26.5 mg, 262 μmol) and cryloyl chloride (14.2 mg, 157 μmol). The mixture was stirred at 0° C. for 0.5 hours. Upon completion, the reaction mixture was concentrated under reduced pressure to obtain a residue, which was purified by prep-TLC (SiO₂, petroleum ether/EtOAc=2:1) to give MY-1A (51.0 mg, 89% yield) as a white solid.

HRMS ESI-TOF m/z calculated for C₂₂H₁₉BrN₃O₂ [M+H]+ 436.0655. Found 436.0646.

¹H NMR (400 MHz, CDCl₃): δ 10.47 (br s, 1H), 8.81 (d, J=3.6 Hz, 1H), 8.43 (d, J=4.0 Hz, 1H), 8.16 (d, J=8.0 Hz, 1H), 7.54-7.42 (m, 3H), 7.29-7.25 (m, 4H+CHCl3), 6.53 (d, J=16.3 Hz, 1H), 6.48-6.18 (m, 1H), 5.98-5.62 (m, 1H), 5.38 (d, J=8.0 Hz, 1H), 4.64 (t, J=9.3 Hz, 1H), 4.59-4.40 (m, 1H), 4.33-4.24 (m, 1H).

### Synthesis of MY-1B

(2R,3S)-1-acryloyl-3-(4-bromophenyl)-N-(quinolin-8-yl)azetidine-2-carboxamide (MY-1Bx2) was prepared in analogous fashion from ent-S1 (Maetani et al., 2017).

HRMS ESI-TOF m/z calculated for C₂₂H₁₉BrN₃O₂ [M+H]⁺ 436.0655. Found 436.0649.

¹H NMR (400 MHz, CDCl₃): δ 10.47 (s, 1H), 8.80 (d, J=4.0 Hz, 1H), 8.42 (d, J=4.0 Hz, 1H), 8.15 (d, J=8.0 Hz, 1H), 7.52-7.40 (m, 3H), 7.30-7.25 (m, 4H+CHCl3), 6.64-6.15 (m, 2H), 5.90-5.64 (m, 1H), 5.37 (d, J=8.0 Hz, 1H), 4.63 (t, J=9.3 Hz, 1H), 4.57-4.38 (m, 1H), 4.33-4.22 (m, 1H).

### Synthesis of MY-3A

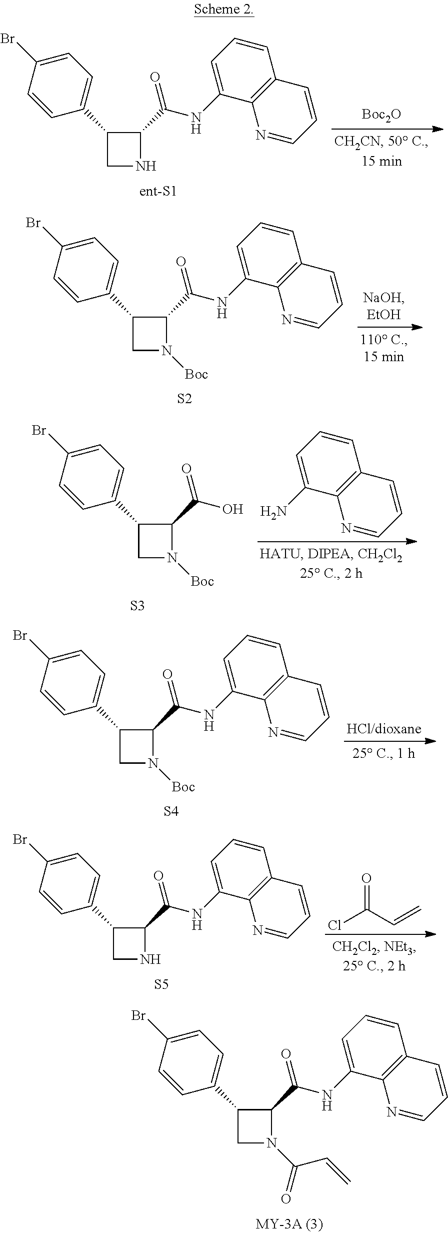

### Step 1: Synthesis of (2S,3S)-3-(4-bromophenyl)-1-(tert-butoxycarbonyl)azetidine-2-carboxylic acid (S3)

To a solution of ent-S1 (450 mg, 1.18 mmol) in acetonitrile (3 mL) was added Boc₂O (308 mg, 1.41 mmol), and the resulting mixture was stirred at 50° C. for 15 min. Upon completion, the reaction mixture was filtered over Celite and concentrated under reduced pressure to give S2 (600 mg, crude) as a yellow oil. To a solution of S2 (450 mg, 933 μmol) in ethanol (3 mL) was added sodium hydroxide (373 mg, 9.33 mmol), and the resulting mixture was stirred at 110° C. for 15 min. Upon completion, the mixture was diluted with water and washed with dichloromethane (2×100 mL). The resulting aqueous solution was acidified with HCl (1 M) to adjust pH to 5-6, exhaustively extracted with i-PrOH/CHCl3 (3:7, 5×60 mL), dried over anhydrous sodium sulfate, filtered, and concentrated under reduced pressure to give S3 (330 mg, 99% yield over two steps) as a white solid.

LC-MS m/z calculated for C₁₅H₁₉BrNO₄ [M+H]⁺ 356.0. Found 356.0.

### Step 2: (2S,3S)-3-(4-bromophenyl)-N-(quinolin-8-yl)azetidine-2-carboxamide (S5)

To a solution of S3 (330 mg, 926 μmol) in dichloromethane (4 mL) were added diisopropylethylamine (239 mg, 1.85 mmol) and 8-aminoquinoline (23.7 mg, 262 μmol), followed by HATU (705 mg, 1.85 mmol). The resulting mixture was stirred at 25° C. for 2 hours. Upon completion, the reaction mixture was concentrated under reduced pressure to give a residue, which was purified by prep-TLC (SiO₂, petroleum ether/EtOAc=2:1) to give S4 (200 mg) as a yellow solid used directly in the next step. To a solution of S4 (100 mg) in dichloromethane (1 mL) was added HCl/dioxane (4 M, 1 mL). and the resulting mixture was stirred at 25° C. for 1 hour. Upon completion, the reaction mixture was partitioned between ethyl acetate (40 mL) and brine (30 mL). The water layer was extracted with i-PrOH/CHCl3 (3:7, 5×20 mL), then the organic phase was dried over sodium sulfate, filtered, and concentrated under reduced pressure to give S5 (90.0 mg, 51% yield over two steps) as an off-white solid. LC-MS m/z calculated for C₁₉H₁₇BrN₃O [M+H]⁺ 382.1. Found 382.1.

### Step 3: (2S,3S)-1-acryloyl-3-(4-bromophenyl)-N-(quinolin-8-yl)azetidine-2-carboxamide (MY-3A)(3)

To a solution of S5 (80.0 mg, 209 μmol) in dichloromethane (1 mL) were added triethylamine (42.4 mg, 419 μmol) and acryloyl chloride (37.9 mg, 419 μmol). The mixture was stirred at 25° C. for 2 hours. Upon completion, the reaction mixture was concentrated under reduced pressure. The residue was purified by preparative TLC (SiO₂, petroleum ether/EtOAc=2:1) to give MY-3A (34.0 mg, 37% yield) as a white solid.

HRMS ESI-TOF m/z calculated for C₂₂H₁₉BrN₃O₂ [M+H]⁺ 436.0655. Found 436.0649.

¹H NMR (400 MHz, CDCl₃): δ 11.13-10.67 (m, 1H), 8.95-8.84 (m, 1H), 8.83-8.75 (m, 1H), 8.16 (d, J=8.0 Hz, 1H), 7.60-7.50 (m, 4H), 7.45 (dd, J=8.2, 4.2 Hz, 1H), 7.33-7.28 (m, 2H), 6.54 (br d, J=16.9 Hz, 1H), 6.42-6.25 (m, 1H), 5.91-5.75 (m, 1H), 5.18-4.95 (m, 1H), 4.76-4.55 (m, 1H), 4.43-4.14 (m, 2H).

### Synthesis of MY-3B

(2S,3S)-1-acryloyl-3-(4-bromophenyl)-N-(quinolin-8-yl)azetidine-2-carboxamide (MY-3B) (4) was prepared in the analogous fashion to S1.

HRMS ESI-TOF m/z calculated for C₂₂H₁₉BrN₃O₂ [M+H]⁺ 436.0655. Found 436.0643.

¹H NMR (400 MHz, CDCl₃): δ 10.95 (br s, 1H), 8.97-8.69 (m, 2H), 8.15 (d, J=8.2 Hz, 1H), 7.55 (d, J=4.7 Hz, 2H), 7.52 (d, J=8.1 Hz, 2H), 7.45 (dd, J=8.3, 4.2 Hz, 1H), 7.28 (d, J=8.2 Hz, 2H), 6.53 (d, J=16.9 Hz, 1H), 6.41-6.27 (m, 1H), 5.90-5.75 (m, 1H), 5.15-5.02 (m, 1H), 4.73-4.59 (m, 1H), 4.37-4.21 (m, 2H).

### Synthesis of MY-11B

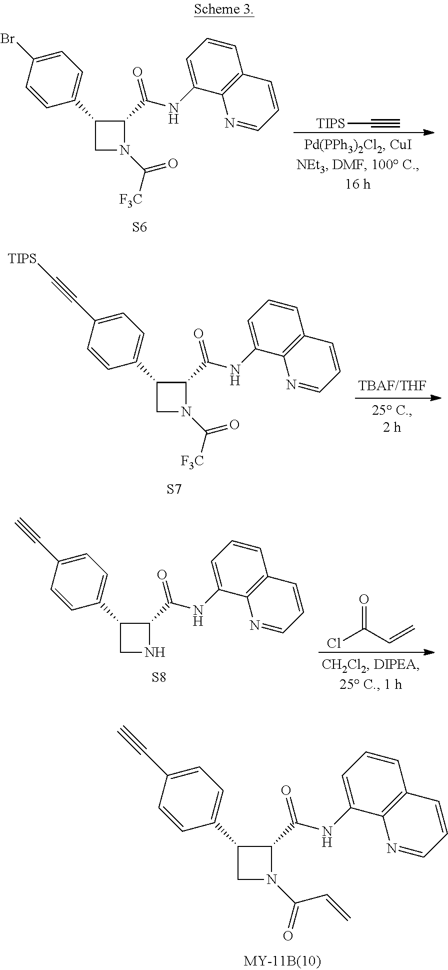

### Step 1: (2R,3S)—N-(quinolin-8-yl)-1-(2,2,2-trifluoroacetyl)-3-(4-((triisopropylsilyl)ethynyl)phenyl)azetidine-2-carboxamide (S7)

To a solution of S6 (690 mg, 1.44 mmol) and (triisopropylsilyl)acetylene (789 mg, 4.33 mmol) in N,N-dimethyl formamide (2 mL) were added copper(I) iodide (27.5 mg, 144 μmol), Pd(PPh₃)₂Cl₂ (101 mg, 144 μmol) and triethylamine (292 mg, 2.89 mmol). The mixture was stirred at 100° C. for 16 hours under nitrogen atmosphere. Upon completion, the reaction mixture was partitioned between ethyl acetate (60 mL) and brine (40 mL). The water layer was extracted with ethyl acetate (40 mL×3). The organic layers were combined, dried over anhydrous sodium sulfate, filtered, and concentrated under reduced pressure to give a residue, which was purified by column chromatography (SiO₂, petroleum ether/EtOAc=100:1 to 1:1) to give S7 (500 mg, 59% yield) as a white solid.

LC-MS m/z calculated for C₃₂H₃₇F₃N₃O₂Si [M+H]⁺ 580.3. Found 580.4.

¹H NMR (400 MHz, CD₃OD, mixture of rotamers): δ 10.18-9.69 (m, 1H), 8.84-8.65 (m, 1H), 8.52-8.33 (m, 1H), 8.14 (dd, J=8.3, 1.7 Hz, 1H), 7.53-7.39 (m, 3H), 7.34-7.22 (m, 3H+CHCl3), 7.17 (d, J=8.0 Hz, 1H), 5.66-5.38 (m, 1H), 4.91-4.39 (m, 3H), 1.11-0.91 (m, 21H).

### Step 2: (2R,3S)-1-acryloyl-3-(4-ethynylphenyl)-N-(quinolin-8-yl)azetidine-2-carboxamide (MY-11B) (10)

To a solution of S7 (500 mg, 862 μmol) in tetrahydrofuran (8.6 mL) was added tetrabutylammonium fluoride (1 M in THF, 8.6 mL). The mixture was stirred at 25° C. for 2 hours. Upon completion, the reaction mixture was concentrated in vacuo to give the residue. The residue was purified by prep-TLC (SiO₂, petroleum ether/ethyl acetate=0:1) to obtain S8 (250 mg) as a white solid. To a solution of S8 (100 mg, 305 μmol) in dichloromethane (2 mL) were added diisopropylethylamine (79.0 mg, 611 μmol) and acryloyl chloride (55.3 mg, 611 μmol). The mixture was stirred at 25° C. for 1 hour. Upon completion, the reaction mixture was concentrated under reduced pressure to give a residue. The residue was purified by preparative TLC (SiO₂, petroleum ether/EtOAc=1:2) and prep-HPLC (column: Waters Xbridge 150 mm×25 mm×5 μm; mobile phase: [water (10 mM NH₄HCO₃)—CH₃CN]; B %: 35%-68%, 8 min). It was further separated by SFC (Column: Chiralpak AD-3 50×4.6 mm I.D., 3 μm Mobile phase: Phase A: CO₂, and Phase B: i-PrOH (0.05% diethylamine); Gradient elution: 40% i-PrOH (0.05% diethylamine) in CO2; Flow rate: 3 mL/min; Column Temp: 35° C.; Back Pressure: 100 Bar) to obtain MY-11B (52.0 mg, 53% yield over two steps) as a white solid.

HRMS ESI-TOF m/z calculated for C₂₄H₂₀N₃O₂ [M+H]+ 382.1550. Found 382.1542.

¹H NMR (400 MHz, DMSO-d₆, mixture of rotamers): δ 10.4-10.2 (m, 1H), 8.90 (d, J=4.0 Hz, 1H), 8.37 (d, J=8.0 Hz, 1H), 8.21-8.12 (m, 1H), 7.65-7.56 (m, 2H), 7.45 (t, J=8.0 Hz, 1H), 7.40-7.34 (m, 2H), 7.26-7.16 (m, 2H), 6.62-6.10 (m, 2H), 5.84 (d, J=9.9 Hz, 1H), 5.70-5.40 (m, 1H), 4.65-4.55 (m, 1H), 4.50-4.22 (m, 2H), 4.10-4.00 (m, 1H).

### Synthesis of MY-11A

(2S,3R)-1-acryloyl-3-(4-bromophenyl)-N-(quinoline-8-yl)azetdine-2-carboxamide (MY-11Ax9) was prepared in a fashion that is analogous to ent-S-6.

HRMS ESI-TOF m/z calculated for C₂₄H₂₀N₃O₂[M+H]⁺ 382.1550; Found 382.1540.

¹H NMR (400 MHz, CD₃OD): δ 8.85 (dd, J=4.3, 1.7 Hz, 1H), 8.29 (dd, J=8.3, 1.7 Hz, 1H), 8.26-8.11 (m, 1H), 7.60 (dd, J=8.3, 1.3 Hz, 1H), 7.55 (dd, J=8.3, 4.2 Hz, 1H), 7.44 (t, J=8.0 Hz, 1H), 7.39 (d, J=7.9 Hz, 2H), 7.21 (d, J=7.8 Hz, 2H), 6.83-6.23 (m, 2H), 6.09-5.36 (m, 2H), 4.80-4.40 (m, 4H), 3.36 (s, 1H).

### Synthesis of MY-45A and MY-45B P-P,3

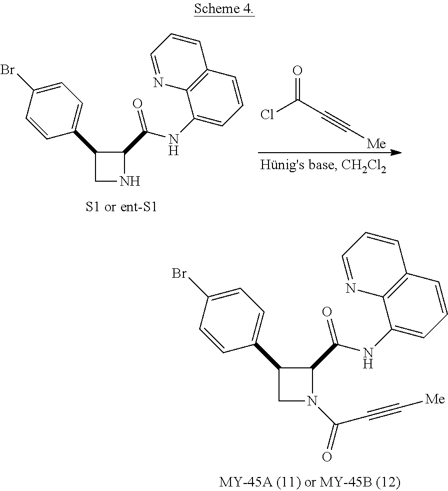

General procedure A: preparation of but-2-ynoyl chloride solution. PCl₅ (115 mg, 0.55 mmol, 1.1 equiv) was added to an ice-cold suspension of but-2-ynoic acid (42.0 mg, 0.50 mmol, 1.0 equiv) in dichloromethane (1 mL). The ice bath was removed and the reaction mixture was stirred at room temperature until it turned into a clear solution (typically 30 minutes to 1 hour).

General procedure B: butynamide formation. But-2-ynoyl chloride (0.5 M solution in dichloromethane, 1.5 equiv), prepared according to general procedure A, was slowly added to a solution of the corresponding amine (1.0 equiv) and Hunig's base (3.0 equiv) in dichloromethane (0.05 M) at 0° C. The reaction was allowed to warm to room temperature and stirred until complete consumption of starting material (as monitored by TLC). The reaction was quenched by addition of sat. aq. NaHCO₃ and was extracted with EtOAc (3×). The combined organic layers were dried over anhydrous sodium sulfate, filtered, and concentrated under reduced pressure. The residue was purified by flash chromatography and preparative TLC as indicated.

### Synthesis of (2S,3R)-3-(4-bromophenyl)-1-(but-2-ynoyl)-N-(quinolin-8-yl)azetidine-2-carboxamide (MY-45A) (11)

Following general procedure B using Si (16.9 mg, 0.044 mmol, 1.0 equiv). (Maetani et al., 2017) Purification by flash column chromatography (SiO₂, dichloromethane/acetone=100:0 to 10:1) followed by preparative TLC (SiO₂, CHCl₃/acetone=9:1) provided MY-45A as a white foam (9.9 mg, 50% yield).

HRMS ESI-TOF m/z calculated for C₂₃H₁₉BrN₃O₂ [M+H]⁺ 448.0655. Found 448.0648.

¹H NMR (400 MHz, CDCl₃, mixture of rotamers): δ 10.54 (s, 0.6H), 10.35 (s, 0.4H), 8.95-8.77 (m, 1H), 8.40 (d, J=7.4 Hz, 1H), 8.21-8.09 (m, 1H), 7.57-7.38 (m, 3H), 7.32-7.20 (m, 4H), 5.40 (d, J=9.7 Hz, 0.6H), 5.29 (d, J=9.7 Hz, 0.4H), 4.69-4.53 (m, 1H), 4.53-4.32 (m, 1H), 4.32-4.15 (m, 1H), 2.10 (s, 1H), 1.79 (s, 2H).

### Synthesis of (2R,3S)-3-(4-bromophenyl)-1-(but-2-ynoyl)-N-(quinolin-8-yl)azetidine-2-carboxamide (MY-45B) (12)

Following general procedure B using ent-S1 (18.7 mg, 0.049 mmol, 1.0 equiv). (Maetani et al., 2017) Purification by flash column chromatography (SiO₂, dichloromethane/acetone=100:0 to 10:1) followed by preparative TLC (SiO₂, CHCl₃/acetone=9:1) provided MY-45B as a white foam (10.3 mg, 47% yield).

HRMS ESI-TOF m/z calculated for C₂₃H₁₉BrN₃O₂ [M+H]⁺ 448.0655. Found 448.0644.

¹H NMR (400 MHz, CDCl₃, mixture of rotamers): δ 10.55 (s, 0.6H), 10.35 (s, 0.4H), 8.98-8.74 (m, 1H), 8.40 (d, J=7.4 Hz, 1H), 8.22-7.98 (m, 1H), 7.73-7.40 (m, 3H), 7.30-7.19 (m, 4H), 5.40 (d, J=9.7 Hz, 0.6H), 5.29 (d, J=9.7 Hz, 0.4H), 4.72-4.50 (m, 1H), 4.52-4.32 (m, 1H), 4.32-4.08 (m, 1H), 2.10 (s, 1H), 1.80 (s, 2H).

### Synthesis of Tryptoline Probes

EV-96, EV-97, EV-98, EV-99 were prepared as reported previously (Vinogradova et al., 2020).

### Synthesis of methyl (1R,3S)-2-acryloyl-1-(benzo[d][1,3]dioxol-5-yl)-2,3,4,9-tetrahydro-1H-pyrido[3,4-b]indole-3-carboxylate (EV-96) (5)

HRMS ESI-TOF m/z calculated for C₂H₂₁N₂O₅ [M+H]⁺ 405.1445. Found 405.1441.

¹H NMR (400 MHz, CD₃OD): δ 7.44 (d, J=7.8 Hz, 1H), 7.33-7.17 (m, 1H), 7.12-7.03 (m, 1H), 7.00 (t, J=7.4 Hz, 1H), 6.95-6.85 (m, 2H), 6.84-6.65 (m, 2H), 6.30-6.04 (m, 2H), 6.00-5.79 (m, 2H), 5.70 (dd, J=10.6, 1.8 Hz, 1H), 5.50-4.95 (m, 1H), 3.73-3.51 (m, 3H), 3.50-3.39 (m, 1H), 3.26-3.14 (m, 1H), 1 exchangeable proton not observed.

### Synthesis of methyl (1S,3R)-2-acryloyl-1-(benzo[d][1,3]dioxol-5-yl)-2,3,4,9-tetrahydro-1H-pyrido[3,4-b]indole-3-carboxylate (EV-97) (6)

HRMS ESI-TOF m/z calculated for C₂₃H₂₁N₂O₅ [M+H]⁺ 405.1445. Found 405.1450.

¹H NMR (400 MHz, CD₃OD): δ 7.44 (d, J=8.0 Hz, 1H), 7.32-7.15 (m, 1H), 7.11-7.04 (m, 1H), 7.00 (t, J=7.4 Hz, 1H), 6.95-6.86 (m, 2H), 6.85-6.68 (m, 2H), 6.30-6.05 (m, 2H), 6.00-5.79 (m, 2H), 5.70 (dd, J=10.6, 1.8 Hz, 1H), 5.55-4.95 (m, 1H), 3.70-3.51 (m, 3H), 3.50-3.37 (m, 1H), 3.28-3.10 (m, 1H), 1 exchangeable proton not observed.

### Synthesis of methyl (1S,3S)-2-acryloyl-1-(benzo[d][1,3]dioxol-5-yl)-2,3,4,9-tetrahydro-1H-pyrido[3,4-b]indole-3-carboxylate (EV-98) (7)

HRMS ESI-TOF m/z calculated for C₂H₂₁N₂O₅ [M+H]⁺ 405.1445. Found 405.1444.

¹H NMR (400 MHz, CD₃OD): δ 7.52 (d, J=7.8 Hz, 1H), 7.28 (d, J=8.0 Hz, 1H), 7.15-7.09 (m, 1H), 7.08-7.03 (m, 1H), 7.02-6.81 (m, 2H), 6.80 (s, 1H), 6.73-6.39 (m, 2H), 6.49-6.20 (m, 1H), 5.90 (s, 2H), 5.82 (d, J=8.0 Hz, 1H), 5.69-5.23 (m, 1H), 3.66-3.50 (m, 1H), 3.13 (s, 3H), 3.07-2.96 (m, 1H), 1 exchangeable proton not observed.

### Synthesis of methyl (1R,3R)-2-acryloyl-1-(benzo[d][1,3]dioxol-5-yl)-2,3,4,9-tetrahydro-1H-pyrido[3,4-b]indole-3-carboxylate (EV-99) (8)

HRMS ESI-TOF m/z calculated for C₂H₂₁N₂O₅ [M+H]⁺ 405.1445. Found 405.1442.

¹H NMR (400 MHz, CD₃OD): δ 7.52 (d, J=8.0 Hz, 1H), 7.28 (d, J=8.0 Hz, 1H), 7.15-7.09 (m, 1H), 7.08-7.02 (m, 1H), 7.02-6.84 (m, 2H), 6.80 (s, 1H), 6.73-6.65 (m, 1H), 6.62-6.53 (m, 1H), 6.38-6.21 (m, 1H), 5.90 (s, 2H), 5.87-5.78 (m, 1H), 5.68-5.25 (m, 1H), 3.66-3.56 (m, 1H), 3.13 (s, 3H), 3.09-2.97 (m, 1H), 1 exchangeable proton not observed.

### Synthesis of WX-01-10

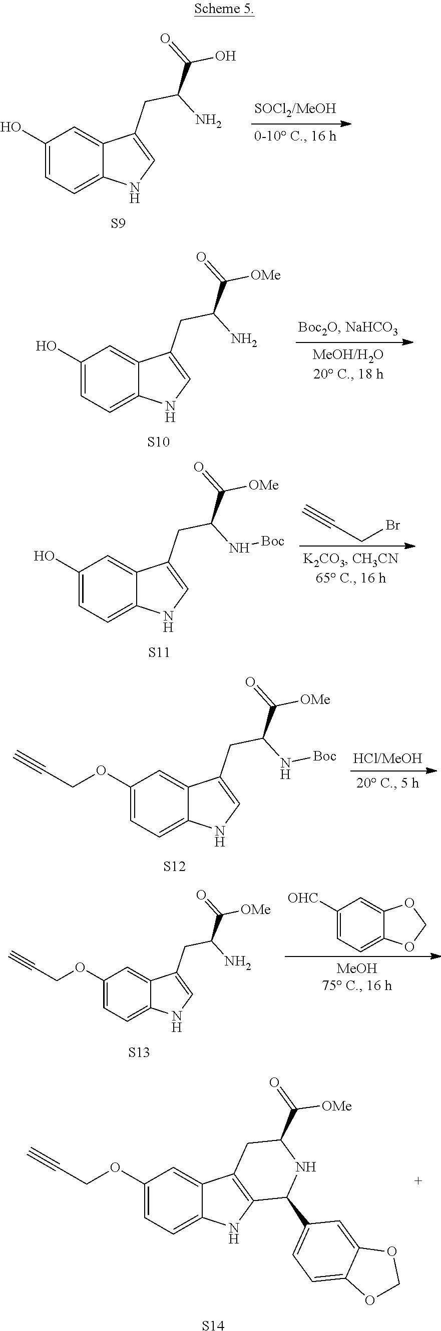

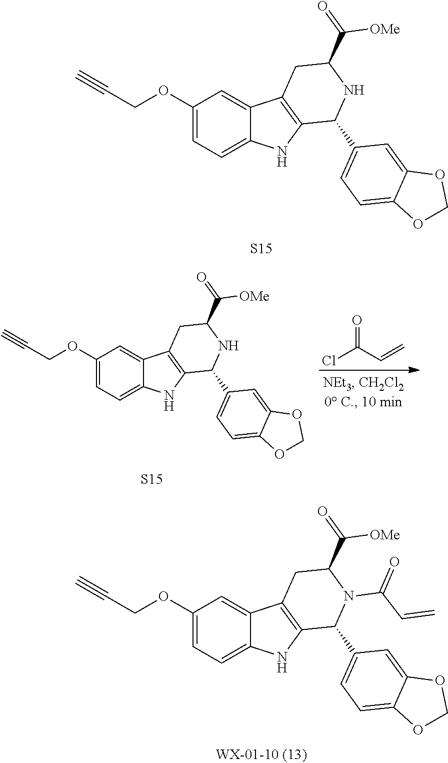

### Steps 1 and 2: methyl (S)-2-((tert-butoxycarbonyl)amino)-3-(5-hydroxy-1H-indol-3-yl)propanoate (S11))

To a solution of S9 (2.00 g, 9.08 mmol) in methanol (20 mL) was added dropwise thionyl chloride (2.00 g, 16.8 mmol, 1.22 mL) at 0° C. The mixture was warmed and stirred at 40° C. for 16 hours. The reaction was monitored by LC-MS. The reaction mixture was concentrated under reduced pressure to give S10 (1.90 g, crude) as a yellow oil, which was used for next step without purification. To a solution of S10 (1.90 g, crude) in methanol (20 mL) and water (5 mL) were added Boc₂O (3.54 g, 16.2 mmol, 3.73 mL) and sodium bicarbonate (2.04 g, 24.3 mmol). The mixture was stirred at 20° C. for 16 hours and the reaction was monitored by LC-MS. Upon completion, the reaction mixture was diluted with water (50 mL) and extracted with ethyl acetate (3×40 mL). The organic layers were combined, dried over anhydrous sodium sulfate, filtered, and concentrated under reduced pressure to give Si 1 (3.00 g, crude) as a yellow solid, which was used in the next step without further purification. LC-MS m/z calculated for C₁₇H₂₃N₂O₅ [M+H]+ 335.2. Found 335.2.

¹H NMR (400 MHz, CDCl₃): δ 7.93 (br s, 1H), 7.21 (d, J=8.7 Hz, 1H), 6.97 (dd, J=7.9, 2.4 Hz, 2H), 6.77 (dd, J=8.7, 2.4 Hz, 1H), 5.13-5.00 (m, 1H), 4.69-4.55 (m, 1H), 3.68 (s, 3H), 3.49 (d, J=4.5 Hz, 1H), 3.25-3.17 (m, 2H), 1.57 (s, 9H).

### Step 3: Synthesis of methyl (S)-2-((tert-butoxycarbonyl)amino)-3-(5-(prop-2-yn-1-yloxy)-1H-indol-3-yl)propanoate (S12)

To a solution of S11 (2.00 g, crude) in acetonitrile (30 mL) were added potassium carbonate (2.48 g, 17.9 mmol) and propargyl bromide (934 mg, 6.28 mmol, 677 PL, 80% w/w in toluene). The mixture was stirred at 85° C. for 16 hours. The reaction was monitored by LC-MS. The reaction mixture was extracted with EtOAc (3×40 mL). The organic layers were combined, dried over anhydrous sodium sulfate, filtered, and concentrated under reduced pressure to give a residue, which was purified by column chromatography (SiO₂, petroleum ether/EtOAc=10:1 to 5:1) to give S12 (2.30 g, quant.) as a white solid.

LC-MS m/z calculated for C₁₅H₁₇N₂O₃ [M-Boc+H]⁺ 273.1. Found 273.1.

¹H NMR (400 MHz, CDCl₃): δ 8.00 (br s, 1H), 7.29-7.24 (m, 1H+CHCl3), 7.11 (d, J=2.4 Hz, 1H), 7.01 (d, J=2.5 Hz, 1H), 6.92 (dd, J=8.8, 2.4 Hz, 1H), 5.13-5.05 (m, 1H), 4.73 (d, J=2.4 Hz, 2H), 4.68-4.61 (m, 1H), 3.68 (s, 3H), 3.24 (d, J=5.6 Hz, 2H), 2.52 (t, J=2.4 Hz, 1H), 1.42 (s, 9H).

### Steps 4 and 5: Synthesis of Compounds S14 and S15

To a solution of S12 (1.30 g, 3.49 mmol) in methanol (13 mL) was added HCl (4 M in MeOH, 9 mL). The mixture was stirred at 20° C. for 5 hours. The reaction was monitored by LC-MS. Upon completion, the reaction mixture was concentrated under reduced pressure to give S13 (1.00 g, crude) as a yellow oil, which was used in the next step without purification. To a solution of S13 (900 mg, 3.31 mmol) in methanol (15 mL) was added piperonal (595 mg, 3.97 mmol). The mixture was stirred at 75° C. for 16 hours. The reaction was monitored by LC-MS. Upon completion, the reaction mixture was diluted with water (20 mL) to adjust the pH to 8-9, then extracted with EtOAc (3×20 mL). The organic layers were combined, dried over anhydrous sodium sulfate, filtered, and concentrated under reduced pressure to give a residue. The residue was purified by column chromatography (SiO₂, petroleum ether/EtOAc=1:0 to 2:1) to give S14 (130 mg, 7.7% yield) and S15 (130 mg, 7.2% yield) as yellow solids.

### Synthesis of (1S,3S)-1-(benzo[d][1,3]dioxol-5-yl)-6-(prop-2-yn-1-yloxy)-2,3,4,9-tetrahydro-1H-pyrido [3,4-b]indole-3-carboxylate (S14)

LC-MS m/z calculated for C₂₃H₂₁N₂O₅ [M+H]⁺ 405.1. Found 405.2.

¹H NMR (400 MHz, CDCl₃): δ 7.34 (br s, 1H), 7.13 (d, J=8.7 Hz, 1H), 7.09 (d, J=2.5 Hz, 1H), 6.87 (ddd, J=8.4, 5.6, 2.1 Hz, 2H), 6.84-6.75 (m, 2H), 5.95 (s, 2H), 5.21-5.10 (m, 1H), 4.79-4.68 (m, 2H), 3.95 (dd, J=11.1, 4.3 Hz, 1H), 3.82 (s, 3H), 3.17 (ddd, J=15.0, 4.2, 1.8 Hz, 1H), 2.97 (ddd, J=15.0, 11.1, 2.5 Hz, 1H), 2.52 (t, J=2.4 Hz, 1H), 1 exchangeable proton not observed.

### Synthesis of methyl (1R,3S)-1-(benzo[d][1,3]dioxol-5-yl)-6-(prop-2-yn-1-yloxy)-2,3,4,9-tetrahydro-1H-pyrido [3,4-b]indole-3-carboxylate (S15)

LC-MS m/z calculated for C₂₃H₂₁N₂O₅ [M+H]⁺ 405.1. Found 405.2.

¹H NMR (400 MHz, CDCl₃): δ 7.46 (br s, 1H), 7.15 (d, J=8.7 Hz, 1H), 7.14-7.08 (m, 1H), 6.93-6.78 (m, 1H), 6.75 (s, 3H), 5.99-5.89 (m, 2H), 5.35-5.29 (m, 1H), 4.74 (dd, J=3.5, 2.4 Hz, 2H), 3.98 (t, J=6.0 Hz, 1H), 3.72 (s, 3H), 3.22 (ddd, J=15.3, 5.5, 1.3 Hz, 1H), 3.09 (ddd, J=15.4, 6.6, 1.5 Hz, 1H), 2.52 (t, J=2.4 Hz, 1H), 1 exchangeable proton not observed.

### Synthesis of methyl (1R,3S)-2-acryloyl-1-(benzo[d][1,3]dioxol-5-yl)-6-(prop-2-yn-1-yloxy)-2,3,4,9-tetra-hydro-1H-pyrido[3,4-b]indole-3-carboxylate (WX-01-10) (13)

To a solution of S15 (80.00 mg, 198 μmol) in dichloromethane (2 mL) were added triethylamine (40.0 mg, 396 μmol, 55.1 uL) and acryloyl chloride (17.9 mg, 198 μmol, 16.1 uL). The mixture was stirred at 0° C. for 10 min and the reaction was monitored by LC-MS. Upon completion, the reaction mixture was concentrated under reduced pressure to give a residue, which was purified by prep-HPLC (column: Phenomenex Luna C18 150*25 mm*10 um; mobile phase: [water (0.225% FA)-ACN]; B %: 39/6-69%, 10 min) and preparatory TLC (SiO₂, petroleum ether/EtOAc=1:1) to give WX-01-10 (18.0 mg, 20% yield) as a white solid.

HRMS ESI-TOF m/z calculated for C₂₆H₂₃N₂O₆ [M+H]⁺ 459.1551. Found 459.1551.

¹H NMR (400 MHz, CDCl₃): δ 7.64 (br s, 1H), 7.17 (d, J=8.8 Hz, 1H), 7.08 (d, J=2.4 Hz, 1H), 6.89 (dd, J=8.8, 2.4 Hz, 1H), 6.84 (br s, 1H), 6.82-6.74 (m, 2H), 6.57 (br dd, J=16.6, 10.8 Hz, 1H), 6.30 (dd, J=1 6.6, 1.6 Hz, 1H), 6.10 (s, 1H), 5.93 (br d, J=6.4 Hz, 2H), 5.66 (br d, J=10.6 Hz, 1H), 5.10 (br s, 1H), 4.73 (d, J=2.4 Hz, 2H), 3.66 (s, 3H), 3.54 (br d, J=15.2 Hz, 1H), 3.25 (br s, 1H), 2.52 (t, J=2.4 Hz, 1H).

### Synthesis of WS-01-12

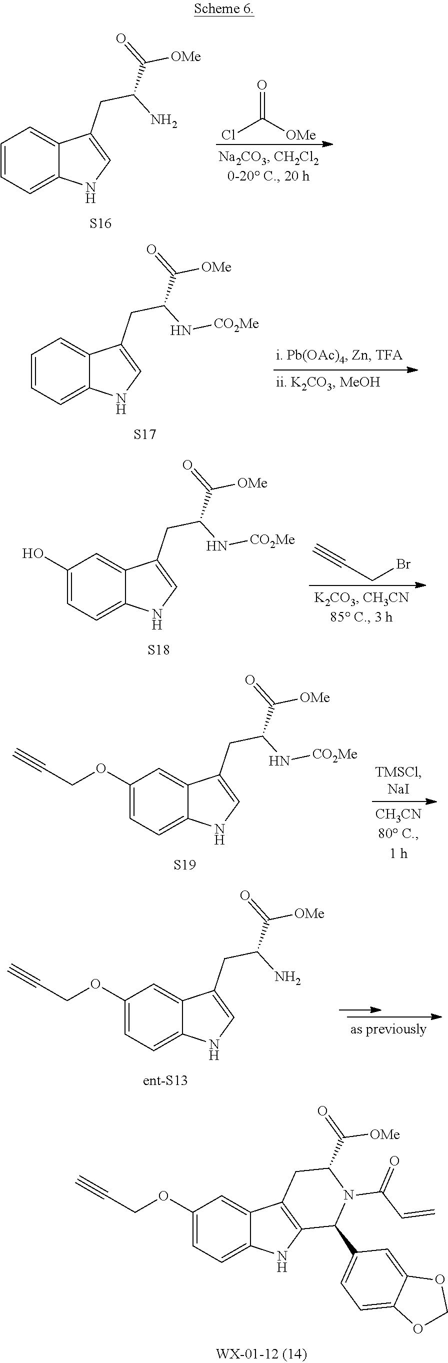

### Step 1: methyl (methoxycarbonyl)-D-tryptophanate (S17)

To a precooled (0° C.) solution of S16 (1.05 g, 4.12 mmol, HCl) in dichloromethane (20 mL) were added sodium carbonate (0.584 g, 6.18 mmol) and methyl chloroformate (0.655 g, 6.18 mmol, 0.48 mL). The mixture was allowed to gradually warm to 20° C. overnight (20 h). The reaction mixture was diluted with water (100 mL) and dichloromethane (50 mL). The organic layer was washed sequentially with water, sat. aq. sodium bicarbonate, and brine. The organic layer was dried over anhydrous sodium sulfate, filtered, and concentrated under reduced pressure to give S17 (1.14 g, 4.12 mmol, quant.), which was used in the next step without further purification.

LC-MS m/z calculated for C₁₄H₁₇N₂O₄ [M+H]⁺ 277.1. Found 277.1.

¹H NMR (400 MHz, CDCl₃) δ 8.10 (br s, 1H), 7.54 (d, J=7.9 Hz, 1H), 7.36 (dt, J=8.1, 0.9 Hz, 1H), 7.20 (ddd, J=8.2, 7.0, 1.3 Hz, 1H), 7.13 (ddd, J=8.0, 7.0, 1.1 Hz, 1H), 7.01 (d, J=2.2 Hz, 1H), 5.23 (d, J=8.3 Hz, 1H), 4.71 (td, J=5.8, 5.8 Hz, 1H), 3.68 (s, 3H), 3.67 (s, 3H), 3.31 (d, J=5.5 Hz, 2H).

### Step 2: methyl (R)-3-(5-hydroxy-1H-indol-3-yl)-2-((methoxycarbonyl)amino)propanoate (S18)

S17 (1.14 g, 4.12 mmol) was dissolved in trifluoroacetic acid (12 mL) and the solution was stirred at 20° C. for 2.5 h. Next, the reaction mixture was cooled to 12° C. (1,4 dioxane dry ice bath) and a solution of lead tetraacetate (4.02 g, 9.08 mmol; best results were obtained using fresh Strem Chemicals batch) in dichloromethane (80 mL) was added over 10 min. The brown mixture was stirred for 1.5 h at the same temperature before adding zinc (1.35 g, 20.6 mmol) and warming the reaction to 20° C. over 45 min (the reaction becomes amber in color). The reaction was diluted with water (100 mL) and stirred vigorously over 30 min before extracting with dichloromethane (3×50 mL). The combined organic layers were filtered through a silica plug and concentrated under reduced pressure. The resulting brown oil was dissolved in methanol (20 mL) and treated with potassium carbonate (0.375 g, 2.71 mmol) overnight (18 h) at 20° C. to solvolyze any trifluoroacetate ester formed over previous steps. The resulting solution was diluted in 50% sat. aq. NaCl and extracted with dichloromethane (3×50 mL). The combined organic layers were dried over anhydrous sodium sulfate, filtered, and concentrated under reduced pressure. The residue was purified by column chromatography (SiO₂, hexanes/EtOAc=1:1 with 0.1% acetic acid) to give S18 as a tan oil/foam (0.605 g, 4.13 mmol, 50%).

LC-MS m/z calculated for C₁₄H₁₇N₂O₅ [M+H]⁺ 293.3. Found 293.0.

¹H NMR (400 MHz, CDCl₃) δ 8.10 (br s, 1H), 7.17 (dd, J=8.8, 2.6 Hz, 1H), 6.99-6.92 (m, 2H), 6.77 (dd, J=8.6, 2.4 Hz, 1H), 5.43-5.31 (m, 1H), 4.67 (ddd, J=6.4, 6.3, 6.3 Hz, 1H), 3.67 (s, 3H), 3.65 (s, 3H), 3.20 (d, J=5.7 Hz, 2H), 1 exchangeable proton not observed.

### Step 3: methyl (R)-2-((methoxycarbonyl)amino)-3-(5-(prop-2-yn-1-yloxy)-1H-indol-3-yl)propanoate (S19)

To a solution of S18 (690 mg, 2.36 mmol) in acetonitrile (20 mL) were added potassium carbonate (979 mg, 7.08 mmol) and propargyl bromide (351 mg, 2.36 mmol, 254 μL, 80% w/w in toluene), and the mixture was stirred at 85° C. for 3 hours. The reaction was monitored by TLC and LC-MS. The reaction mixture was diluted with water (100 mL) and extracted with ethyl acetate (50 mL×3). The combined organic layers were dried over anhydrous sodium sulfate, filtered, and concentrated under reduced pressure. The resulting residue was purified by column chromatography (SiO₂, petroleum ether/EtOAc=1:1 to 1:1) to give S19 (550 mg, 1.66 mmol, 71% yield) as a yellow oil.

LC-MS m/z calculated for C₁₇H₁₉N₂O₅ [M+H]⁺ 331.1. Found 331.1.

### Step 4: methyl (R)-2-amino-3-(5-(prop-2-yn-1-yloxy)-1H-indol-3-yl)propanoate (ent-S13)

To a solution of S19 (400 mg, 1.21 mmol) in acetonitrile (4 mL) was added trimethylchlorosilane (263 mg, 2.42 mmol, 307 μL) and sodium iodide (363 mg, 2.42 mmol). The mixture was stirred at 80° C. for 1 hour. This reaction was monitored by LC-MS. The reaction mixture was diluted with water (100 mL) and extracted with EtOAc (3×30 mL). The combined organic layers were dried over anhydrous sodium sulfate, filtered, and concentrated under reduced pressure to give ent-S13 (510 mg, crude) as a yellow oil. It was used in next step directly without purification.

The remaining transformations were performed as described previously for WX-01-10.

### Synthesis of methyl (1S,3R)-2-acryloyl-1-(benzo[d][1,3]dioxol-5-yl)-6-(prop-2-yn-1-yloxy)-2,3,4,9-tetra-hydro-1H-pyrido[3,4-b]indole-3-carboxylate (WX-01-12) (14)

HRMS ESI-TOF m/z calculated for C₂₆H₂₃N₂O₆ [M+H]⁺ 459.1551. Found 459.1552.

¹H NMR (400 MHz, CDCl₃): δ 7.74 (br s, 1H), 7.15 (d, J=8.8 Hz, 1H), 7.07 (d, J=2.4 Hz, 1H), 6.87 (dd, J=8.8, 2.5 Hz, 1H), 6.85-6.70 (m, 3H), 6.56 (dd, J=16.7, 10.5 Hz, 1H), 6.29 (dd, J=16.7, 1.8 Hz, 1H), 6.09 (s, 1H), 5.99-5.86 (m, 2H), 5.64 (d, J=10.5 Hz, 1H), 5.17-5.00 (m, 1H), 4.72 (d, J=2.4 Hz, 2H), 3.64 (s, 3H), 3.59-3.45 (m, 1H), 3.39-3.03 (m, 1H), 2.51 (t, J=2.4 Hz, 1H).

### Synthesis of WS-02-23

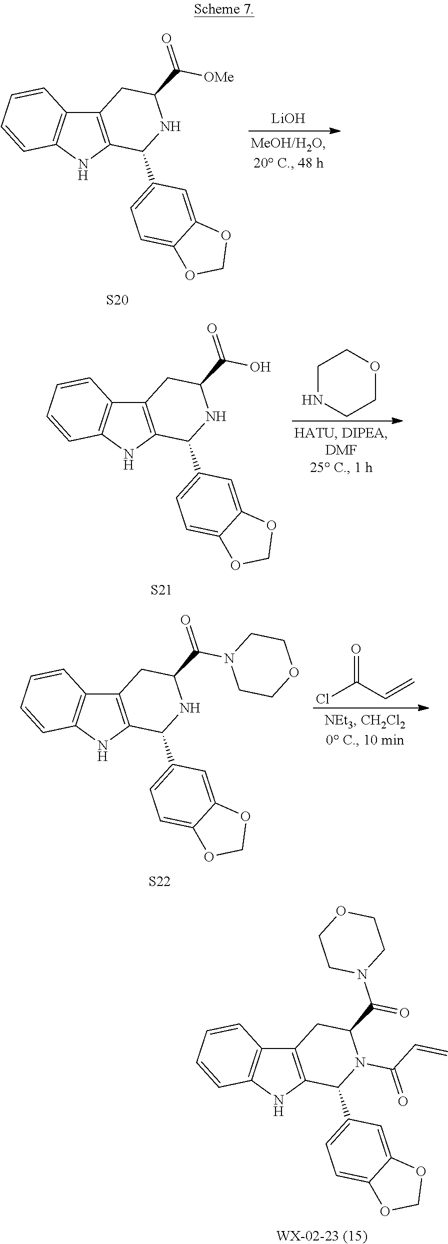

### Step 1: (1R,3S)-1-(benzo[d][1,3]dioxol-S-yl)-2,3,4,9-tetrahydro-1H-pyrido[3,4-b]indole-3-carboxylic acid (S21)

To a solution of S20 (500 mg, 1.43 mmol) (Vinogradova et al., 2020) in methanol (10 mL) were added lithium hydroxide monohydrate (71.9 mg, 1.71 mmol) and water (257 mg, 14.3 mmol, 257 μL). The mixture was stirred at 20° C. for 48 hours. The reaction was monitored by LC-MS. The reaction mixture was concentrated under reduced pressure to give a residue, which was suspended in toluene (10 mL) and concentrated under reduced pressure to remove residual water. S21 (500 mg, 97% yield), obtained as a white solid was used in the next step without further purification.

LC-MS m/z calculated for C₁₉H₁₇N₂O₄ [M+H]+ 337.1. Found 337.1.

¹H NMR (400 MHz, DMSO-d₆): δ 7.40 (d, J=8.0 Hz, 1H), 7.30-7.10 (m, 3H), 7.05-6.89 (m, 2H), 6.80 (d, J=8.0 Hz, 1H), 6.74 (m, 1H), 6.60-6.54 (m, 1H), 5.96 (s, 2H), 5.15 (m, 1H), 3.15-3.06 (m, 1H), 2.95-2.85 (m, 1H), 2.65-2.54 (m, 2H).

### Step 2: ((1R,3S)-1-(benzo[d][1,3]dioxol-S-yl)-2,3,4,9-tetrahydro-1H-pyrido[3,4-b]indol-3-yl)(morpholino)methanone (S22)

To a solution of S21 (350 mg, 1.04 mmol) in N,N-dimethyl formamide (2 mL) were added HATU (594 mg, 1.56 mmol), morpholine (1.81 g, 20.8 mmol, 1.83 mL), and diisopropylethylamine (269 mg, 2.08 mmol, 363 μL). The mixture was stirred at 25° C. for 1 hour and the reaction was monitored by LC-MS. Upon completion, the reaction mixture was diluted with water (20 mL) and extracted with ethyl acetate (3×20 mL). The organic layers were combined, dried over anhydrous sodium sulfate, filtered, and concentrated under reduced pressure to give a residue, which was purified by prep-HPLC (column: Waters Xbridge 150 mm×25 mm×5 μm; mobile phase: [water (10 mM NH₄HCO₃)—CH₃CN]; B %: 26%-59%, 10 min) to give S22 (300 mg, 70% yield) as a white solid.

LC-MS m/z calculated for C₂₃H24N₃O₄[M+H]⁺ 406.2. Found 406.2.

¹H NMR (400 MHz, CD₃OD): δ 7.50 (d, J=8.0 Hz, 1H), 7.29 (d, J=8.0 Hz, 1H), 7.13-7.06 (m, 1H), 7.06-7.00 (m, 1H), 6.80-6.72 (m, 2H), 6.68-6.62 (m, 1H), 5.94 (s, 2H), 5.27 (s, 1H), 3.97 (dd, J=10.0, 5.0 Hz, 1H), 3.78-3.47 (m, 8H), 3.29-3.13 (m, 1H), 3.05-2.90 (m, 1H), 2 exchangeable protons not observed.

### Step 3: 1-((1R,3S)-1-(benzo[d][1,3]dioxol-S-yl)-3-(morpholine-4-carbonyl)-1,3,4,9-tetrahydro-2H-pyrido [3,4-b]indol-2-yl)prop-2-en-1-one (WX-02-23) (15)

To a solution of S22 (70.0 mg, 173 μmol) in dichloromethane (2 mL) were added triethylamine (34.9 mg, 345 μmol, 48.1 μL) and acryloyl chloride (15.6 mg, 173 μmol, 14.1 μL). The mixture was stirred at 0° C. for 10 min and the reaction was monitored by LC-MS. Upon completion, the reaction mixture was concentrated under reduced pressure to give a residue, which was purified by prep-HPLC (column: Waters Xbridge 150 mm×25 mm×5 μm; mobile phase: [water (10 mM NH₄HCO₃)—CH₃CN]; B %: 28%-58%, 10 min) to give WX-02-23 (23.1 mg, 28% yield) as a white solid.

HRMS ESI-TOF m/z calculated for C₂₆H₆₂N₃O₅ [M+H]⁺ 460.1867. Found 460.1867.

¹H NMR (400 MHz, CD₃OD): δ 7.47 (d, J=7.8 Hz, 1H), 7.27 (d, J=7.9 Hz, 1H), 7.08 (ddd, J=8.2, 7.1, 1.3 Hz, 1H), 7.01 (ddd, J=8.1, 7.1, 1.1 Hz, 1H), 6.98-6.72 (m, 4H), 6.42-6.32 (m, 1H), 6.24 (dd, J=16.6, 1.8 Hz, 1H), 5.96-5.88 (m, 2H), 5.75 (dd, J=10.6, 1.9 Hz, 1H), 5.14-5.01 (m, 1H), 3.68-3.44 (m, 5H), 3.44-3.12 (m, 5H+solvent residual peak), 1 exchangeable proton not observed.

### Synthesis of WX-02-43

Prepared in analogous fashion from ent-S20 (Vinogradova et al., 2020).

### 1-((1S,3R)-1-(benzo[d][1,3]dioxol-5-yl)-3-(morpholine-4-carbonyl)-1,3,4,9-tetrahydro-2H-pyrido [3,4-b]indol-2-yl)prop-2-en-1-one (WX-02-43) (16)

HRMS ESI-TOF m/z calculated for C₂₆H₂₆N₃O₅ [M+H]+ 460.1867. Found 460.1870.

¹H NMR (400 MHz, CD₃OD): δ 7.47 (d, J=7.8 Hz, 1H), 7.27 (d, J=8.0 Hz, 1H), 7.08 (t, J=7.5 Hz, 1H), 7.01 (t, J=7.4 Hz, 1H), 6.98-6.66 (m, 4H), 6.42-6.34 (m, 1H), 6.24 (d, J=16.6 Hz, 1H), 5.93 (s, 2H), 5.76 (dd, J=10.6, 1.4 Hz, 1H), 5.13-5.02 (m, 1H), 3.72-3.44 (m, 5H), 3.44-3.11 (m, 5H+solvent residual peak), 1 exchangeable proton not observed.

What is claimed is:

1. An in vivo engineered protein, comprising: a target protein comprising splicing factor 3B subunit 1 (SF3B1) or proteasome activator complex subunit 1 (PSME1), covalently bound to a small molecule ligand.

2. The in vivo engineered protein of claim 1, wherein the ligand is covalently bound to a ligand binding site of the target protein.

3. The in vivo engineered protein of claim 1 or 2, wherein the ligand is covalently bound to a cysteine residue of the ligand binding site.

4. The in vivo engineered protein of any one of claims 1-3, wherein the ligand comprises an exogenous Michael acceptor.

5. The in vivo engineered of claim 4, wherein the exogenous Michael acceptor is an alkene or alkyne.

6. The in vivo engineered protein of any of claims 1-5, wherein a sulfur atom at the cysteine residue undergoes the Michael reaction with a double bond of the exogenous Michael acceptor.

7. The in vivo engineered protein of any of claims 1-6, wherein the ligand comprises an azetidine or tryptoline.

8. The in vivo engineered protein of any one of claims 1-7, wherein the ligand comprises the structure of Formula (I), or a pharmaceutically acceptable salt or solvate thereof: 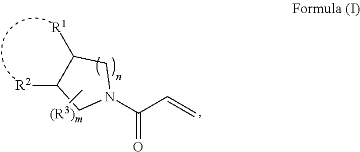
wherein,
R¹ is selected from the group consisting of H, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl;
R² is selected from the group consisting of substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl;
or R¹ and R² together with the atoms to which they are attached form a 5 to 10-membered heterocyclic ring A, optionally having one additional heteroatom moiety selected from NR⁴ or O, wherein A is optionally substituted; and
each R³ is independently H, —C(═O)OR⁵, —C(═O)N(R⁶)₂, —S(═O)₂R⁶, —S(═O)₂N(R⁶)₂, —N(R⁶)C(═O)R⁶, —N(R⁶)S(═O)₂R⁶, substituted or unsubstituted C₁-C₆ alkyl, substituted or unsubstituted aryl, or substituted or unsubstituted heteroaryl;
each R⁴ is independently H or C₁-C₆ alkyl;
each R⁵ is independently H, substituted or unsubstituted C₁-C₆alkyl, substituted or unsubstituted C₁-C₆haloalkyl, or substituted or unsubstituted C₁-C₁₀ heteroalkyl;
each R⁶ is independently H, substituted or unsubstituted C₁-C₆alkyl, substituted or unsubstituted C₁-C₆heteroalkyl, substituted or unsubstituted aryl, or substituted or unsubstituted heteroaryl;
or two R⁶ together with the atom to which they are attached form a 5 to 6-membered heterocyclic ring;
n is 0, 1, 2, or 3; and
m is 1, 2, or 3.

9. The in vivo engineered protein of claim 8, wherein R¹ and R² together with the atoms to which they are attached form a 5 to 10-membered heterocyclic ring A, optionally having one additional heteroatom moiety selected from NR⁴ or O, wherein A is optionally substituted.

10. The in vivo engineered protein of claim 8 or 9, wherein ring A is an 8 to 10-membered bicyclic heteroaryl optionally having one additional heteroatom selected from NR⁴.

11. The in vivo engineered protein of claim 8, wherein the ligand has the structure of Formula (II), or a pharmaceutically acceptable salt or solvate thereof: 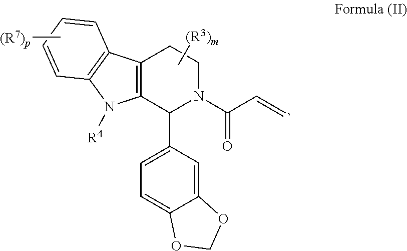
wherein,
each R⁷ is independently H, halogen, cyano, amino, hydroxyl, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, or substituted or unsubstituted C₁-C₆hydroxyalkyl; and
p is 1, 2, 3, or 4.

12. The in vivo engineered protein of claim 8, wherein R¹ is H and R² is selected from the group consisting of substituted or unsubstituted aryl or substituted or unsubstituted heteroaryl.

13. The in vivo engineered protein of claim 8, wherein the ligand has the structure of Formula (III), or a pharmaceutically acceptable salt or solvate thereof: 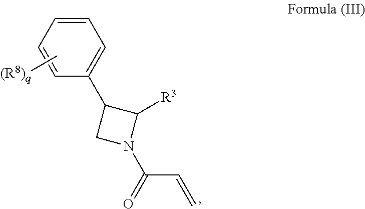
wherein,
each R⁸ is independently H, halogen, cyano, amino, hydroxyl, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, or substituted or unsubstituted C₁-C₆hydroxyalkyl; and
q is 1, 2, 3, or 4.

14. The in vivo engineered protein of any one of claims 8-13, wherein each R³ is independently —C(═O)OR⁵ or —C(═O)N(R⁶)₂.

15. The in vivo engineered protein of any one of claims 8-13, wherein each R³ is independently substituted or unsubstituted aryl, or substituted or unsubstituted heteroaryl.

16. The in vivo engineered protein of any one of claims 8-15, wherein m is 1 or 2.

17. The in vivo engineered protein of any one of claims 1-16, wherein the ligand is selected from: 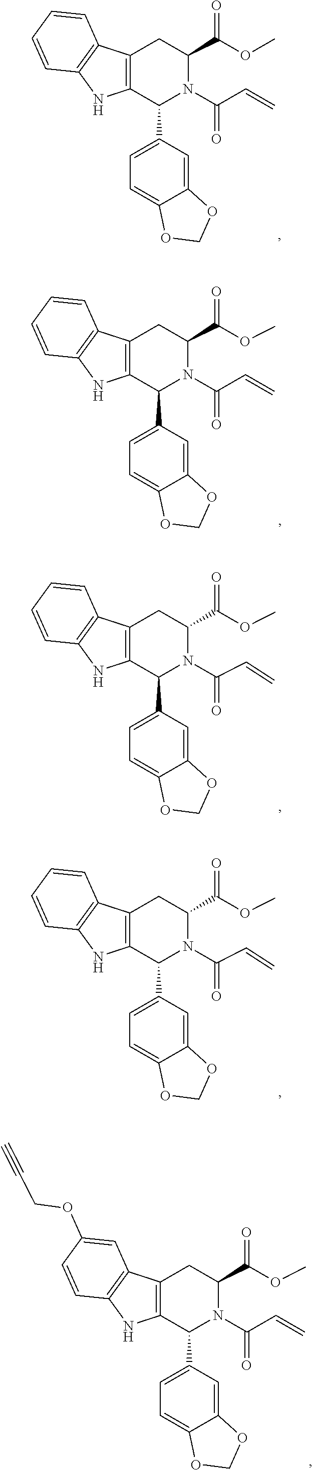 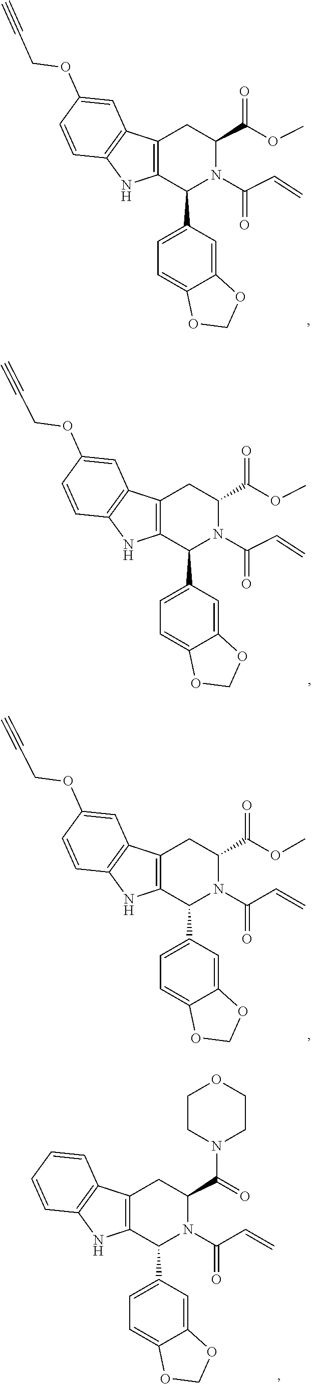 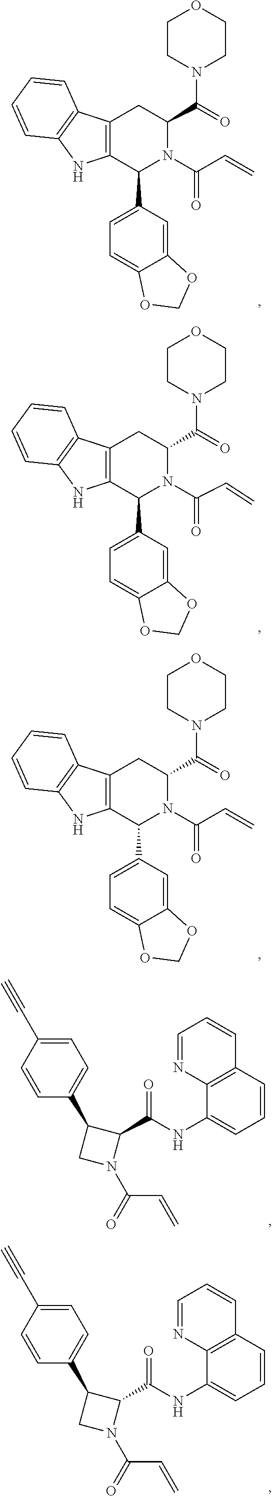 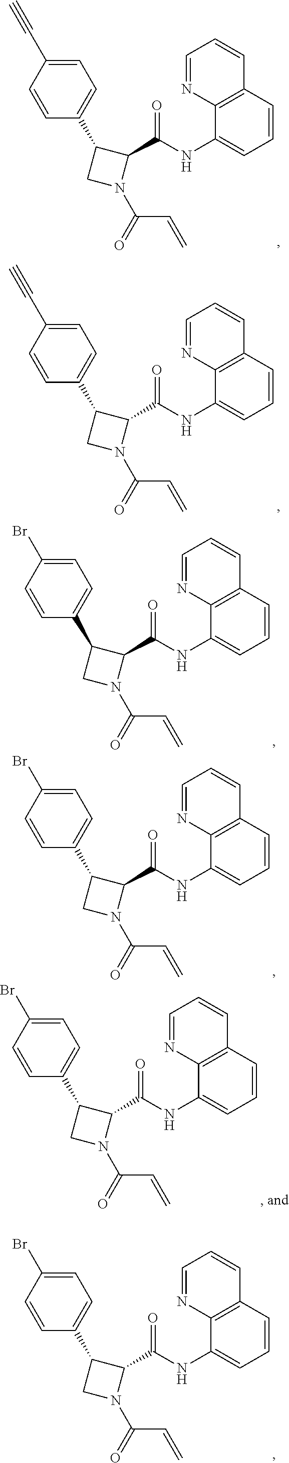
or a pharmaceutically acceptable salt or solvate thereof.

18. The in vivo engineered protein of any one of claims 4-8, wherein the ligand comprises an anti-cancer or immunomodulatory drug.

19. The in vivo engineered protein of claim 1 or 2, wherein the target protein comprises SF3B1.

20. The in vivo engineered protein of any one of claims 1, 2 or 19, wherein the target protein comprises an amino acid sequence having at least 90% sequence identity to SEQ ID NO: 1.

21. The in vivo engineered protein of any one of claims 1, 2, 19, or 20, wherein the ligand is covalently bound at amino acid position 1111 of the SF3B1.

22. The in vivo engineered protein of claim 1 or 2, wherein the target protein comprises PSME1.

23. The in vivo engineered protein of any one of claims 1, 2, or 22, wherein the target protein comprises an amino acid sequence having at least 90% sequence identity to SEQ ID NO: 2.

24. The in vivo engineered protein of any one of claims 1, 2, 22, or 23, wherein the ligand is covalently bound at or near a position on the PSME1 that interfaces with proteasome activator complex subunit 2 (PSME2).

25. The in vivo engineered protein of any one of claims 1, 2, or 22-24, wherein the ligand is covalently bound at amino acid position 22 of the PSME1.

26. The in vivo engineered protein of any one of claims 1, 2, or 22-24, wherein the ligand is covalently bound at amino acid position 106 of the PSME1.

27. The in vivo engineered protein of any one of claims 1-23, comprising a neoantigen.

28. The in vivo engineered protein of any one of claims 1-24, formed in a cell.
